SEQUENCE LISTING (133 sequences)

SEQ ID NO: 1 - protein, 6 aa, synthetic construct
GGSGGS

SEQ ID NO: 2 - protein, 15 aa, synthetic construct
GGGGSGGGGSGGGGS

SEQ ID NO: 3 - protein, 6 aa, synthetic construct
GGSGGS

SEQ ID NO: 4 - protein, 9 aa, synthetic construct
GGGGSGGGG

SEQ ID NO: 5 - protein, 9 aa, synthetic construct
GGGGSGGGG

SEQ ID NO: 6 - protein, 5 aa, synthetic construct
AAAGS

SEQ ID NO: 7 - protein, 125 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSS

SEQ ID NO: 8 - protein, 125 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSWFAYWGQGTLVTVSS

SEQ ID NO: 9 - protein, 110 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIK

SEQ ID NO: 10 - protein, 110 aa, synthetic construct
DIQMTQSPSSLSASVGDRVTITCRSSTGAVTTSNYANWVQQKPGKAPKGLIGGTNKRAPGVPARFSGSGSGTDFTLTISSLQPEDFATYYCALWYSNLWVFGQGTKVEIK

SEQ ID NO: 11 - protein, 110 aa, synthetic construct
DIQMTQSPSSLSASVGDRVTITCRSSTGAVTTSNYANWVQQKPGKAPKGLIGGTNKRAPGVPSRFSGSLIGDKATLTISSLQPEDFATYYCALWYSNLWVFGQGTKVEIK

SEQ ID NO: 12 - protein, 110 aa, synthetic construct
DIQMTQSPSSLSASVGDRVTITCRSSTGAVTTSNYANWVQQKPGKAPKGLIGGTNKRAPGVPSRFSGSLIGTDFTLTISSLQPEDFATYYCALWYSNLWVFGQGTKVEIK

SEQ ID NO: 13 - protein, 6 aa, synthetic construct
STYAMN

SEQ ID NO: 14 - protein, 19 aa, synthetic construct
RIRSKYNNYATYYADSVKD

SEQ ID NO: 15 - protein, 14 aa, synthetic construct
HGNFGNSYVSWFAY

SEQ ID NO: 16 - protein, 14 aa, synthetic construct
HGNFGNSYVSYFAY

SEQ ID NO: 17 - protein, 14 aa, synthetic construct
RSSTGAVTTSNYAN

SEQ ID NO: 18 - protein, 7 aa, synthetic construct
GTNKRAP

SEQ ID NO: 19 - protein, 7 aa, synthetic construct
ALWYSNL

SEQ ID NO: 20 - protein, 120 aa, synthetic construct
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 21 - protein, 106 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 22 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGVRISFDYWGQGTLVTVSS

SEQ ID NO: 23 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDMAVYYCAKGARVSFDYWGQGTLVTVSS

SEQ ID NO: 24 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGVRKGFDYWGQGTLVTVSS

SEQ ID NO: 25 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGVRVSFDYWGQGTLVTVSS

SEQ ID NO: 26 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDAAVYYCAKGVRKKFDYWGQGTLVTVSS

SEQ ID NO: 27 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQGSNQPVTFGQGTKVEIK

SEQ ID NO: 28 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQGSQPPITFGQGTKVEIK

SEQ ID NO: 29 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQGESPPPTFGQGTKVEIK

SEQ ID NO: 30 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQGTTHPITFGQGTKVEIK

SEQ ID NO: 31 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQGQLPPITFGQGTKVEIK

SEQ ID NO: 32 - protein, 119 aa, synthetic construct [sequence omitted]

SEQ ID NO: 33 - protein, 111 aa, synthetic construct
QVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSS

SEQ ID NO: 34 - protein, 94 aa, synthetic construct
QVQLQESGGGLVQPGGSLRLSCVASRSIFSNSIITSADHTNYRDAVKGRFTISRDSAKNTVYLQMNSLKPEDTAVYFCADGRYWGQGTQVTVSS

SEQ ID NO: 35 - protein, 119 aa, synthetic construct
QVQLQESGGGLVQAGGSLRLSCAASESAFNFYTMGWHRQAPGKQRELVASITSGGDTNYANFVEGRFTISRDNAKNTVSLQMNSLNPEDTAIYYCDGKNPRSSSSTWYWGQGTQVTVSS

SEQ ID NO: 36 - protein, 115 aa, synthetic construct
QVQLQESGGGLVQAGGSLRLSCAASTGIFRATVMGWYRQPPGKQRELVARLVSGGSTRYADSVKGRVTISRDNAKNTVYLQINSLKPEDTAVYYCDGIIMNRTYWGQGTQVTVSS

SEQ ID NO: 37 - protein, 117 aa, synthetic construct
QVQLQESGGGLVQAGGSLRLSCASSGSILSMNVMGWYRQAPGKQREMVAQITRIGDTNYSSSMKGRFTISRDNADNTLYLQMNRLEPEDTAVYFCANNNRSTYYYYWGQGTQVTVSS

SEQ ID NO: 38 - protein, 122 aa, synthetic construct
QVQLQESGGGLVQAGGSLRLSCTALRSVGSFNGMGWYRQSPGNQRELVATVTSDTSTTYRDSVKGRFAISRDSAKTVSLQMNNLKPEDTAVYYCADEEGHHLLNRRSTVWYWGQGTQVTVSS

SEQ ID NO: 39 - protein, 122 aa, synthetic construct
QVQLQESGGGLVQAGGSLRLSCTAFRSVGVIDVMGWYRQAPGTQRELVATVTSSSSTTYADSVKGRFAISRDDAKTVSLQMNSLKPEDTAVYYCADDEEGLLNNRRSSVWYWGQGTQVTVSS

SEQ ID NO: 40 - protein, 122 aa, synthetic construct [sequence omitted]

SEQ ID NO: 41 - protein, 116 aa, synthetic construct
QVQLQESGGGLVQAGESLRLSCAASQKIFNNMGWYRQAPGKERELVAEISRGGDTNYAAFTKDRFTISRDNAKNTVYLQMNSLKPEDTAVYYCADGHILPSTVWYWGQGTQVTVSS

SEQ ID NO: 42 - protein, 120 aa, synthetic construct
QVQLQESGGGVVQAGGSLRLSCAPNGGMFVFDAMGWYRQAPGKQRELVATITRSSRRDYADSVKGRFTISKDAAKNTVYLQMNSLKPEDTAVYYCAADGGHNNRSVYYYWGQGTQVTVSS

SEQ ID NO: 43 - protein, 121 aa, synthetic construct
QVQLQESGGGLVQAGGSLRLSCAASGILFSSYLMGWYRQAPGKQRELVAQISADMDTSYVNSVKGRFTISRDNAENTVYLQMNSLEPEDTAVYYCAAFGGKNPRSSTTWYWGQGTQVTVSS

SEQ ID NO: 44 - protein, 124 aa, synthetic construct
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 45 - protein, 117 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTMQGTDYWGQGTQVTVSS

SEQ ID NO: 46 - protein, 119 aa, synthetic construct
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 47 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 48 - protein, 124 aa, synthetic construct
AVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSS

SEQ ID NO: 49 - protein, 127 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 50 - protein, 129 aa, synthetic construct
EVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSS

SEQ ID NO: 51 - protein, 118 aa, synthetic construct
QVQLQESGGGSVQLRLSCAASGYTVRSSYMGWFRQVPGKQREAVAIITSGGTTYYADSVKGRFTISRDNAKNTLYLSLKPEDTAMYYCAGRTGFIGGIWFRDRDYDYWGQGTQVTVSS

SEQ ID NO: 52 - protein, 764 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSAAAGSHHHHHH

SEQ ID NO: 53 - protein, 764 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGSGGGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSAAAGSHHHHHH

SEQ ID NO: 54 - protein, 748 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSAAAGSHHHHHH

SEQ ID NO: 55 - protein, 748 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGSGGGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSAAAGSHHHHHH

SEQ ID NO: 56 - protein, 764 aa, synthetic construct
QVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSGGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKAAAGSHHHHHH

SEQ ID NO: 57 - protein, 764 aa, synthetic construct
QVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSGGGGSGGGGDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGSGGGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSAAAGSHHHHHH

SEQ ID NO: 58 - protein, 748 aa, synthetic construct
QVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSGGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKAAAGSHHHHHH

SEQ ID NO: 59 - protein, 748 aa, synthetic construct
QVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSGGGGSGGGGDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGSGGGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSAAAGSHHHHHH

SEQ ID NO: 60 - protein, 636 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSAAAGSHHHHHH

SEQ ID NO: 61 - protein, 636 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGSGGGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGQVQLQESGGGLVQPGGSLRLSCAASGFTFGSYDMAWVRQAPGKGPEWVSSINSSGGSTEYASSVKGRFTVSRDNAKNMLYLQMDRLKLEDTAVYYCADEGNPSTTWYYEDQGTQVTVSSAAAGSHHHHHH

SEQ ID NO: 62 - protein, 628 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGSGGGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGSGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSAAAGSHHHHHH

SEQ ID NO: 63 - protein, 628 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGSGGGGSDIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSSGGGGSGGGGQVQLQESGGGLVQAGGSLRLSCAASGSIGSINFIFWYRQAPGKQREFVARISRSGRSDYVESVKGRFTISRDNAKNTVYLQMNNLKSEDTAVYYCALGNNWGQGTQVTVSSAAAGSHHHHHH

SEQ ID NO: 64 - protein, 6 aa, synthetic construct
HHHHHH

SEQ ID NO: 65 - protein, 117 aa, synthetic construct
QVQLQESGGGLVQTGGSLRLSCAASGSIFVGNAMGWYRQALGNQRELVAGITSDGTTYYPDSVKGRFTISRDNDKNTIYLQMNSLKPEDTAVYYCNLWPPRIGFASWGQGTQVTVSS

SEQ ID NO: 66 - protein, 116 aa, synthetic construct
QVQLQESGGGLVQAGGSLRLSCWSGTILSSNSMGWYRQAPGKRRELVASISTDGSTNYADSVKGRFTISRDNAKSTVFLQMNSLKPEDTAVYYCNFHPPWRDWGDTYWGTQVTVSS

SEQ ID NO: 67 - protein, 118 aa, synthetic construct
QVQLQESGGGLVQAGGSLRLSCVASGTIFSSGAMAMGWYRQAPGKQREWVAGITGSRTTTYADSVKGRFTISRDNAENTVFLQMNNLKSEDTAVYYCNLWPPSRPDHWGQGTQVTVSS

SEQ ID NO: 68 - protein, 122 aa, synthetic construct
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSS

SEQ ID NO: 69 - protein, 114 aa, synthetic construct
QVQLVESGGGLVQPGGSLTLSCVISGSFSALWAMRWYRQAPGQQRELVASSRGGTTSYADSVKGRFTISRDNSKNTLYLQMNSLRPEDTAVYYCNAIDGHLAYWGQGTLVTVSS

SEQ ID NO: 70 - protein, 126 aa, synthetic construct
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSS

SEQ ID NO: 71 - protein, 126 aa, synthetic construct
QVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSS

SEQ ID NO: 72 - protein, 118 aa, synthetic construct
EVQLVESGGGEVQPGGSLRLSCAASGSGFSASLMSWHRQAPGKQRDLVASITRDGRANYVESVKGRFTISRDNAKNTLYLQMSSLRAEDTAVYYCHAYSFDYPIRSYWGQGTLVTVSS

SEQ ID NO: 73 - protein, 121 aa, synthetic construct
EVQLVESGGGEVQPGGSLRLSCAASGSIFSISIMGWYRQAPGKERELVASTTSSGTTNYVESVKGRFTISRDNAKNTLYLQMSSLRAEDTAVYYCHAYIATTTDRGYRGYWGQGTLVTVSS

SEQ ID NO: 74 - protein, 118 aa, synthetic construct
EVQLVESGGGEVQPGGSLRLSCAASGFTFSSYAMGWFRQAPGKGREWVAAITTSGDTTYYAESVKGRFTISRDNAKNTVYLQMSSLRAEDTAVYYCAAHRGGGVIDYWGQGTLVTVSS

SEQ ID NO: 75 - protein, 125 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 76 - protein, 109 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 77 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 78 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 79 - protein, 803 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSGGGGSEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSGGGGSGGGGSGGGGSGGGGSEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSAAAGSHHHHHH

SEQ ID NO: 80 - protein, 803 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSGGGGSEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSGGGGSGGGGSGGGGSGGGGSEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSAAAGSHHHHHH

SEQ ID NO: 81 - protein, 789 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTMQGTDYWGQGTQVTVSSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTMQGTDYWGQGTQVTVSSAAAGSHHHHHH

SEQ ID NO: 82 - protein, 789 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTMQGTDYWGQGTQVTVSSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTMQGTDYWGQGTQVTVSSAAAGSHHHHHH

SEQ ID NO: 83 - protein, 793 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSGGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGGSGGGGSGGGGSGGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSAAAGSHHHHHH

SEQ ID NO: 84 - protein, 793 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSGGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGGSGGGGSGGGGSGGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSAAAGSHHHHHH

SEQ ID NO: 85 - protein, 123 aa, synthetic construct
QVQLQESGGGLVQPGGSLRLSCAASGSIYSLIAMGWYRQAPGKEHELVATISSGSTTYYADSVKGRFTISRDNAKNTLYLQMNSLKPEDTAMYYCAAYSDRLTDCSNCEADYWGQGTQVTVSH

SEQ ID NO: 86 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 87 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 88 - protein, 635 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 89 - protein, 635 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 90 - protein, 770 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 91 - protein, 770 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 92 - protein, 905 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 93 - protein, 905 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 94 - protein, 1040 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 95 - protein, 1040 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 96 - protein, 770 aa, synthetic construct
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 97 - protein, 770 aa, synthetic construct
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 98 - protein, 1040 aa, synthetic construct
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 99 - protein, 1040 aa, synthetic construct
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 100 - protein, 766 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGSGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGSGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 101 - protein, 766 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGSGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGSGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 102 - protein, 768 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 103 - protein, 768 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 104 - protein, 762 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 105 - protein, 766 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 106 - protein, 774 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 107 - protein, 627 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 108 - protein, 631 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 109 - protein, 639 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 110 - protein, 776 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGSGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGSGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 111 - protein, 776 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGSGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGSGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 112 - protein, 778 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 113 - protein, 778 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 114 - protein, 772 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 115 - protein, 776 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 116 - protein, 784 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 117 - protein, 632 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 118 - protein, 636 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 119 - protein, 644 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 120 - protein, 780 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 121 - protein, 780 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 122 - protein, 772 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKGGGSGGGSEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSGGGSGGGSGGGSGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKGGGSGGGSEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 123 - protein, 780 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGSGGGSGGGSGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 124 - protein, 635 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 125 - protein, 770 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 126 - protein, 640 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 127 - protein, 780 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 128 - protein, 377 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 129 - protein, 512 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGSGGGSGGGSGGGSKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 130 - protein, 382 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 131 - protein, 522 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 132 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 133 - protein, 20 aa, synthetic construct
  VARIANT 1..20: note = This sequence may encompass 1-5 GGGS repeating units
GGGSGGGSGGGSGGGSGGGS

(19) United States
(12) Patent Application Publication
(10) Pub. No.: US 2025/0136690 A1
(43) Pub. Date: May 1, 2025

(54) MULTISPECIFIC BINDING PROTEIN COMPOSITIONS AND USES THEREOF

(71) Applicant: Toreador Therapeutics, Inc., San Francisco, CA (US)

(72) Inventor: Jeanmarie GUENOT, San Francisco, CA (US)

(21) Appl. No.: 18/835,482

(22) PCT Filed: Feb. 3, 2023
(86) PCT No.: PCT/US2023/012344

§ 371 Date: Aug. 2, 2024

Related U.S. Application Data
(60) Provisional application No. 63/307,584, filed on Feb. 7, 2022.

(51) Int. Cl. C07K 16/28; A61K 39/00; A61P 35/00; C07K 16/32

(52) U.S. Cl. CPC C07K 16/2809; A61P 35/00; C07K 16/2863; C07K 16/32; A61K 2039/505; C07K 2317/569; C07K 2317/622; C07K 2317/73; C07K 2317/92

(57) ABSTRACT

Described herein are single chain polypeptide compositions comprising variable domains connected by linkers that fold into a conformation such that the variable domains form two effector cell antigen interacting domains (E) wherein each effector cell antigen interacting domain comprises two variable domains of a single chain variable fragment (EV and EV), and at least one of the effector cell antigen interacting domains is linked to one or more target interacting domains. Also described herein are methods of use of the single chain polypeptides.

### CROSS-REFERENCE

The present application claims the benefit of U.S. Provisional Application No. 63/307,584, filed on Feb. 7, 2022, which is incorporated by reference in its entirety.

### SUMMARY

Disclosed herein are single chain polypeptides comprising variable domains connected by linkers that fold into a conformation such that the variable domains form two effector cell antigen interacting domains (E) wherein each effector cell antigen interacting domain comprises two variable domains of a single chain variable fragment (EV and EV), and at least one of the effector cell antigen interacting domains is linked to one or more target interacting domains. In some embodiments, the target interacting domain binds to a tumor antigen, a ligand, a protein, a lipid, nucleic acid, or a polysaccharide. In some embodiments, the tumor antigen is on a cell in a tumor microenvironment. In some embodiments, the target interacting domain comprises an antibody, an antibody fragment, a ligand, or a cofactor. In some embodiments, the antibody or antibody fragment of the targeting interacting domain comprises a Fab, a Fab′, a F(ab′)₂, a Fv fragment, a single-chain Fv (scFv), a tandem single-chain Fv ((scFv)₂, a dual affinity retargeting antibody, a diabody, a VHH single domain antibody, a TriTac, an Adnectin, an Anticalin, an Avimer, a Fynomer, a Kunitz domain, a knottin, an Affibody, or a DARPin. In some embodiments, the one or more target interacting domains comprises one or more target cell antigen interacting domains that bind to a target cell antigen. In some embodiments, the target cell antigen interacting domain binds to more than one target cell antigen. In some embodiments, the target cell antigen interacting domain is a bispecific or multispecific antibody or antibody fragment. In some embodiments, the target cell antigen interacting domain comprises a VHH single domain antibody (TVHH). In some embodiments, the single chain polypeptide does not contain a fragment crystallizable (Fc) domain. In some embodiments, the two variable domains of the single chain variable fragment (EV and EV) comprise a variable heavy chain of a single chain variable fragment (EVH) and a variable light chain of single chain variable fragment (EVL). In some embodiments, the EVL of the at least one of the effector cell antigen interacting domains that is linked to the one or more target cell antigen interacting domains is linked to at least one TVHH, and the EVH of the one of the effector cell antigen interacting domains that is linked to the one or more target cell antigen interacting domains is linked to at least one TVHH. In some embodiments, the at least one TVHH that is linked to the EVL binds to the same target antigen as the at least one TVHH that is linked to the EVH. In some embodiments, the at least one TVHH that is linked to the EVL binds to the same target antigen and has the same complementarity determining region (CDR) sequences as the at least one TVHH that is linked to the EVH. In some embodiments, the at least one TVHH that is linked to the EVL binds to the same target antigen and have no complementarity determining region (CDR) sequences in common as the at least one TVHH that is linked to the EVH. In some embodiments, the at least one TVHH that is linked to the EVL binds to a different target antigen as compared to the at least one TVHH that is linked to the EVH. In some embodiments, the EVL and the EVH are linked to the same number of TVHHs. In some embodiments, the EVL is linked to a different number of TVHHs as compared to the number of TVHHs that are linked to the EVH. In some embodiments, the EVL of the at least one of the effector cell antigen interacting domains that is linked to the one or more target cell antigen interacting domains is linked to one TVHH, and the EVH of the at least one of the effector cell antigen interacting domains that is linked to the one or more target cell antigen interacting domains is linked to one TVHH. In some embodiments, the EVL of the at least one of the effector cell antigen interacting domains that is linked to the one or more target cell antigen interacting domains is linked to one TVHH, and the EVH of the at least one of the effector cell antigen interacting domains that is linked to the one or more target cell antigen interacting domains is linked to two TVHH. In some embodiments, the EVL of the at least one of the effector cell antigen interacting domains that is linked to the at least one or more target cell antigen interacting domains is linked to one TVHH, and the EVH of the at least one of the effector cell antigen interacting domains that is linked to the one or more target cell antigen interacting domains is linked to three TVHH. In some embodiments, the EVH of the at least one of the effector cell antigen interacting domains that is linked to the one or more target cell antigen interacting domains is linked to one TVHH, and the EVL of the at least one of the effector cell antigen interacting domains that is linked to the one or more target cell antigen interacting domains is linked to two TVHH. In some embodiments, the EVH of the at least one of the effector cell antigen interacting domains that is linked to the one or more target cell antigen interacting domains is linked to one TVHH, and the EVL of the at least one of the effector cell antigen interacting domains that is linked to the one or more target cell antigen interacting domains is linked to three TVHH. In some embodiments, the EVH of the at least one of the effector cell antigen interacting domains that is linked to the one or more target cell antigen interacting domains is linked to two TVHH, and the EVL of the at least one of the effector cell antigen interacting domains that is linked to the one or more target cell antigen interacting domains is linked to two TVHH. In some embodiments, the EVH of the at least one of the effector cell antigen interacting domains that is linked to the one or more target cell antigen interacting domains is linked to three TVHH, and the EVL of the at least one of the effector cell antigen interacting domains that is linked to the one or more target cell antigen interacting domains is linked to three TVHH. In some embodiments, the EVH of the at least one of the effector cell antigen interacting domains that is linked to the one or more target cell antigen interacting domains is linked to four TVHH, and the EVL of the at least one of the effector cell antigen interacting domains that is linked to the one or more target cell antigen interacting domains is linked to four TVHH. In some embodiments, the EVH of the at least one of the effector cell antigen interacting domains that is linked to the one or more target cell antigen interacting domains is linked to five TVHH, and the EVL of the at least one of the effector cell antigen interacting domains that is linked to the one or more target cell antigen interacting domains is linked to five TVHH. In some embodiments, the EVH of the at least one of the effector cell antigen interacting domains that is linked to the one or more target cell antigen interacting domains is linked to six TVHH, and the EVL of the at least one of the effector cell antigen interacting domains that is linked to the one or more target cell antigen interacting domains is linked to six TVHH. In some embodiments, the EVH of the at least one of the effector cell antigen interacting domains that is linked to the one or more target cell antigen interacting domains is linked to seven TVHH, and the EVL of the at least one of the effector cell antigen interacting domains that is linked to the one or more target cell antigen interacting domains is linked to seven TVHH. In some embodiments, the EVH of the at least one of the effector cell antigen interacting domains that is linked to the one or more target cell antigen interacting domains is linked to eight TVHH, and the EVL of the at least one of the effector cell antigen interacting domains that is linked to the one or more target cell antigen interacting domains is linked to eight TVHH. In some embodiments, the at least one of the effector cell antigen interacting domains that is linked to the one or more target cell antigen interacting domains is linked to one target cell antigen interacting domain. In some embodiments, the one or more target cell antigen interacting domain is linked C-terminal to the at least one of the effector cell antigen interacting domains. In some embodiments, the one or more target cell antigen interacting domains is linked N-terminal to the at least one of the effector cell antigen interacting domains. In some embodiments, the at least one of the effector cell antigen interacting domains that is linked to the one or more target cell antigen interacting domains is linked to more than one target cell antigen interacting domain. In some embodiments, all of the more than one target cell antigen interacting domains bind to the same target antigen. In some embodiments, all of the more than one target cell antigen interacting domains bind to the same target antigen and all have the same CDR sequences. In some embodiments, all of the more than one target cell antigen interacting domains bind to the same target antigen and at least two of the more than one target cell antigen interacting domains have no CDR sequences in common. In some embodiments, at least two of the more than one target cell antigen interacting domains bind to different target antigens. In some embodiments, the at least one of the effector cell antigen interacting domains that is linked to the one or more target cell antigen interacting domains is linked to two target cell antigen interacting domains. In some embodiments, the two target cell antigen interacting domains are linked C-terminal to the one of the effector cell antigen interacting domains. In some embodiments, the two target cell antigen interacting domains are linked N-terminal to the one of the effector cell antigen interacting domains. In some embodiments, the at least one target one of the effector cell antigen interacting domains that is linked to the one or more target cell antigen interacting domains is linked to three target cell antigen interacting domains. In some embodiments, the three target cell antigen interacting domains are linked C-terminal to the one of the effector cell antigen interacting domains. In some embodiments, the three target cell antigen interacting domains are linked N-terminal to the one of the effector cell antigen interacting domains. In some embodiments, the at least one target one of the effector cell antigen interacting domains that is linked to the one or more target cell antigen interacting domains is linked to four target cell antigen interacting domains. In some embodiments, the four target cell antigen interacting domains are linked C-terminal to the one of the effector cell antigen interacting domains. In some embodiments, the four target cell antigen interacting domains are linked N-terminal to the one of the effector cell antigen interacting domains. In some embodiments, the variable domains and the one or more target cell antigen interacting domains are ordered according to the following N-terminal to C-terminal arrangements in which (-) is the linker: TVHH-EV-EV-EV-EV; EV-EV-EV-EV-TVHH; TVHH-TVHH-EV-EV-EV-EV; EV-EV-EV-EV-TVHH-TVHH; TVHH-TVHH-TVHH-EV-EV-EV-EV; EV-EV-EV-EV-TVHH-TVHH-TVHH; TVHH-TVHH-TVHH-TVHH-EV-EV-EV-EV; EV-EV-EV-EV-TVHH-TVHH-TVHH-TVHH; TVHH-EV-EV-EV-EV-TVHH; TVHH-EV-EV-EV-EV-TVHH-TVHH; TVHH-TVHH-EV-EV-EV-EV-TVHH; TVHH-EV-EV-EV-EV-TVHH-TVHH-TVHH; TVHH-TVHH-TVHH-EV-EV-EV-EV-TVHH; TVHH-TVHH-EV-EV-EV-EV-TVHH-TVHH; TVHH-TVHH-TVHH-EV-EV-EV-EV-TVHH-TVHH-TVHH; TVHH-TVHH-TVHH-TVHH-EV-EV-EV-EV-TVHH-TVHH-TVHH-TVHH; TVHH-TVHH-TVHH-TVHH-TVHH-EV-EV-EV-EV-TVHH-TVHH-TVHH-TVHH-TVHH; TVHH-TVHH-TVHH-TVHH-TVHH-TVHH-EV-EV-EV-EV-TVHH-TVHH-TVHH-TVHH-TVHH-TVHH; TVHH-TVHH-TVHH-TVHH-TVHH-TVHH-TVHH-EV-EV-EV-EV-TVHH-TVHH-TVHH-TVHH-TVHH-TVHH-TVHH; or TVHH-TVHH-TVHH-TVHH-TVHH-TVHH-TVHH-TVHH-EV-EV-EV-EV-TVHH-TVHH-TVHH-TVHH-TVHH-TVHH-TVHH-TVHH. In some embodiments, the variable domains and the one or more target cell antigen interacting domains are ordered according to the following N-terminal to C-terminal arrangements in which (-) is the linker: TVHH-TVHH-EV-EV-EV-EV-TVHH-TVHH; or TVHH-EV-EV-EV-EV-TVHH. In some embodiments, the variable domains and the one or more target cell antigen interacting domains are ordered according to the following N-terminal to C-terminal arrangements in which (-) is the linker: TVHH-TVHH-EV-EV-EV-EV-TVHH-TVHH. In some embodiments, the variable domains and the one or more target cell antigen interacting domains are ordered according to the following N-terminal to C-terminal arrangements in which (-) is the linker: TVHH-EV-EV-EV-EV-TVHH. In some embodiments, the variable domains and the one or more target cell antigen interacting domains are ordered according to the following N-terminal to C-terminal arrangements in which (-) is the linker: TVHH-TVHH-TVHH-TVHH-TVHH-TVHH-TVHH-TVHH-EV-EV-EV-EV-TVHH-TVHH-TVHH-TVHH-TVHH-TVHH-TVHH-TVHH. In some embodiments, the variable domains and the one or more target cell antigen interacting domains are ordered according to the following N-terminal to C-terminal arrangements in which (-) is the linker: TVHH-EVL-EVH-EVL-EVH; TVHH-EVH-EVL-EVH-EVL; EVL-EVH-EVL-EVH-TVHH; EVH-EVL-EVH-EVL-TVHH; TVHH-TVHH-EVL-EVH-EVL-EVH; TVHH-TVHH-EVH-EVL-EVH-EVL; EVL-EVH-EVL-EVH-TVHH-TVHH; EVH-EVL-EVH-EVL-TVHH-TVHH; TVHH-TVHH-TVHH-EVL-EVH-EVL-EVH; TVHH-TVHH-TVHH-EVH-EVL-EVH-EVL; EVL-EVH-EVL-EVH-TVHH-TVHH-TVHH; EVH-EVL-EVH-EVL-TVHH-TVHH-TVHH; TVHH-TVHH-TVHH-TVHH-EVL-EVH-EVL-EVH; TVHH-TVHH-TVHH-TVHH-EVH-EVL-EVH-EVL; EVL-EVH-EVL-EVH-TVHH-TVHH-TVHH-TVHH; EVH-EVL-EVH-EVL-TVHH-TVHH-TVHH-TVHH; TVHH-EVL-EVH-EVL-EVH-TVHH; TVHH-EVH-EVL-EVH-EVL-TVHH; TVHH-EVL-EVH-EVL-EVH-TVHH-TVHH; TVHH-EVH-EVL-EVH-EVL-TVHH-TVHH; TVHH-TVHH-EVL-EVH-EVL-EVH-TVHH; TVHH-TVHH-EVH-EVL-EVH-EVL-TVHH; TVHH-EVL-EVH-EVL-EVH-TVHH-TVHH-TVHH; TVHH-EVH-EVL-EVH-EVL-TVHH-TVHH-TVHH; TVHH-TVHH-TVHH-EVL-EVH-EVL-EVH-TVHH; TVHH-TVHH-TVHH-EVH-EVL-EVH-EVL-TVHH; TVHH-TVHH-EVL-EVH-EVL-EVH-TVHH-TVHH; or TVHH-TVHH-EVH-EVL-EVH-EVL-TVHH-TVHH. In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH; TVHH-TVHH-EVH-EVL-EVH-EVL-TVHH-TVHH; TVHH-EVL-EVH-EVL-EVH-TVHH; or TVHH-EVH-EVL-EVH-EVL-TVHH. In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH; or TVHH-TVHH-EVH-EVL-EVH-EVL-TVHH-TVHH. In some embodiments, the variable domains and the one or more target cell antigen interacting domains are ordered according to the following N-terminal to C-terminal arrangements in which (-) is the linker: TVHH-EVL-EVH-EVL-EVH-TVHH; or TVHH-EVH-EVL-EVH-EVL-TVHH. In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH. In some embodiments, the variable domains and the one or more target cell antigen interacting domains are ordered according to the following N-terminal to C-terminal arrangements in which (-) is the linker: TVHH-TVHH-EVH-EVL-EVH-EVL-TVHH-TVHH. In some embodiments, the variable domains and the one or more target cell antigen interacting domains are ordered according to the following N-terminal to C-terminal arrangements in which (-) is the linker: TVHH-EVL-EVH-EVL-EVH-TVHH. The single chain polypeptide of claim 53, wherein the variable domains and the one or more target cell antigen interacting domains are ordered according to the following N-terminal to C-terminal arrangements in which (-) is the linker: TVHH-EVH-EVL-EVH-EVL-TVHH. In some embodiments, the linkers that connect the two effector cell antigen interacting domains (E) to each other comprise internal linkers. In some embodiments, the effector cell antigen interacting domain that is not connected directly to the one or more target cell antigen interacting domains comprises a terminal linker that connects EVL and EVH. In some embodiments, each of the internal linkers is 6-8 amino acids in length. In some embodiments, the terminal linker is 15 or 16 amino acids in length. In some embodiments, each of the effector cell antigen interacting domains bind to an effector cell antigen. In some embodiments, the effector cell antigen comprises CD3, CD16a, or Death Receptor 5 (DR5). In some embodiments, the CD3 is CD3 delta, CD3 gamma, or CD3 epsilon. In some embodiments, the effector cell antigen is on an effector cell. In some embodiments, the effector cell comprises a T cell, NK cell, or a macrophage. In some embodiments, the target cell antigen interacting domains that interact with a target cell (T) that binds to the target cell antigen. In some embodiments, the target cell antigen is clustered in a lipid raft when on a cell associated with a disease state and not clustered in a lipid raft when on a cell that is not associated with a disease state. In some embodiments, the target cell antigen is clustered when on a cell associated with a disease state and not clustered when on a cell that is not associated with a disease state. In some embodiments, the target cell antigens are 100 nm or less when on a cell associated with a disease state and not within 100 nm when on a cell that is not associated with a disease state. In some embodiments, the target cell antigens are 75 nm or less when on a cell associated with a disease state and not within 75 nm when on a cell that is not associated with a disease state. In some embodiments, the target cell antigens are 50 nm or less when on a cell associated with a disease state and not within 50 nm when on a cell that is not associated with a disease state. In some embodiments, the target cell antigens are 25 nm or less when on a cell associated with a disease state and not within 25 nm when on a cell that is not associated with a disease state. In some embodiments, the target cell antigens are 10 nm or less when on a cell associated with a disease state and not within 10 nm when on a cell that is not associated with a disease state. In some embodiments, the target cell antigens are 5 nm or less when on a cell associated with a disease state and not within 5 nm when on a cell that is not associated with a disease state. In some embodiments, the target cell antigens are 2 nm or less when on a cell associated with a disease state and not within 2 nm when on a cell that is not associated with a disease state. In some embodiments, the target cell antigens are in close proximity when on a cell associated with a disease state and not within close proximity when on a cell that is not associated with a disease state. In some embodiments, the target cell antigen is a dimer, trimer, tetramer, or oligomer when on a cell associated with a disease state and a monomer or dimer when on a cell that is not associated with a disease state. In some embodiments, the target cell antigen is part of a cell signaling complex when on a cell associated with a disease state and not part of the same cell signaling complex when on a cell that is not associated with a disease state. In some embodiments, the target cell antigen is part of a cell signaling complex when on a cell associated with a disease state and not part of a cell signaling complex when on a cell that is not associated with a disease state. In some embodiments, the target cell antigen comprises CD33, FAP, EGFR, HER2, or EpCAM. In some embodiments, the cell associated with a disease state is a myeloid-cell, fibroblast, or cancer cell.

Disclosed herein are single chain polypeptides comprising variable domains connected by linkers that fold into a conformation such that the variable domains form one or more effector cell antigen interacting domains (E) wherein the effector cell antigen interacting domain comprises two variable domains of a single chain variable fragment (EV and EV), and the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain. Disclosed herein are single chain polypeptides comprising variable domains connected by linkers that fold into a conformation such that the variable domains form two effector cell antigen interacting domains (E) wherein each effector cell antigen interacting domain comprises two variable domains of a single chain variable fragment (EV and EV), and one of the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain. In some embodiments, the two variable domains of a single chain variable fragment (EV and EV) comprise a variable heavy chain of a single chain variable fragment (EVH) and a variable light chain of single chain variable fragment (EVL). In some embodiments, the one of the effector cell antigen interacting domains that is connected to the one or more target cell antigen interacting domains is connected to one target cell antigen interacting domain. In some embodiments, the one target cell antigen interacting domain is connected C-terminal to the one of the effector cell antigen interacting domains. In some embodiments, the one target cell antigen interacting domains is connected N-terminal to the one of the effector cell antigen interacting domains. In some embodiments, the one of the effector cell antigen interacting domains that is connected to the one or more target cell antigen interacting domains is connected to two target cell antigen interacting domains. In some embodiments, the two-target cell antigen interacting domains are connected C-terminal to the one of the effector cell antigen interacting domains. In some embodiments, the two-target cell antigen interacting domains are connected N-terminal to the one of the effector cell antigen interacting domains. In some embodiments, the one of the effector cell antigen interacting domains that is connected to the one or more target cell antigen interacting domains is connected to three target cell antigen interacting domains. In some embodiments, the three-target cell antigen interacting domains are connected C-terminal to the one of the effector cell antigen interacting domains. In some embodiments, the three-target cell antigen interacting domains are connected N-terminal to the one of the effector cell antigen interacting domains. In some embodiments, the one of the effector cell antigen interacting domains that is connected to the one or more target cell antigen interacting domains is connected to four target cell antigen interacting domains. In some embodiments, the four-target cell antigen interacting domains are connected C-terminal to the one of the effector cell antigen interacting domains. In some embodiments, the four-target cell antigen interacting domains are connected N-terminal to the one of the effector cell antigen interacting domains. In some embodiments, the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to one TVHH. In some embodiments, the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to two TVHH. In some embodiments, the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to three TVHH. In some embodiments, the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to two TVHH. In some embodiments, the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to three TVHH. In some embodiments, the EVH of the one of the effector cell antigen interacting domains that is connected to the one or more target cell antigen interacting domains is connected to two TVHH, and the EVL of the one of the effector cell antigen interacting domains that is connected to the one or more target cell antigen interacting domains is connected to two TVHH. In some embodiments, the variable domains and the one or more target cell antigen interacting domains are ordered according to the following N-terminal to C-terminal arrangements in which (-) is the linker: TVHH-EV-EV-EV-EV; EV-EV-EV-EV-TVHH; TVHH-TVHH-EV-EV-EV-EV; EV-EV-EV-EV-TVHH-TVHH; TVHH-TVHH-TVHH-EV-EV-EV-EV; EV-EV-EV-EV-TVHH-TVHH-TVHH; TVHH-TVHH-TVHH-TVHH-EV-EV-EV-EV; EV-EV-EV-EV-TVHH-TVHH-TVHH-TVHH; TVHH-EV-EV-EV-EV-TVHH; TVHH-EV-EV-EV-EV-TVHH-TVHH; TVHH-TVHH-EV-EV-EV-EV-TVHH; TVHH-EV-EV-EV-EV-TVHH-TVHH-TVHH; TVHH-TVHH-TVHH-EV-EV-EV-EV-TVHH; or TVHH-TVHH-EV-EV-EV-EV-TVHH-TVHH. In some embodiments, the variable domains and the one or more target cell antigen interacting domains are ordered according to the following N-terminal to C-terminal arrangements in which (-) is the linker: TVHH-EVL-EVH-EVL-EVH; TVHH-EVH-EVL-EVH-EVL; EVL-EVH-EVL-EVH-TVHH; EVH-EVL-EVH-EVL-TVHH; TVHH-TVHH-EVL-EVH-EVL-EVH; TVHH-TVHH-EVH-EVL-EVH-EVL; EVL-EVH-EVL-EVH-TVHH-TVHH; EVH-EVL-EVH-EVL-TVHH-TVHH; TVHH-TVHH-TVHH-EVL-EVH-EVL-EVH; TVHH-TVHH-TVHH-EVH-EVL-EVH-EVL; EVL-EVH-EVL-EVH-TVHH-TVHH-TVHH; EVH-EVL-EVH-EVL-TVHH-TVHH-TVHH; TVHH-TVHH-TVHH-TVHH-EVL-EVH-EVL-EVH; TVHH-TVHH-TVHH-TVHH-EVH-EVL-EVH-EVL; EVL-EVH-EVL-EVH-TVHH-TVHH-TVHH-TVHH; EVH-EVL-EVH-EVL-TVHH-TVHH-TVHH-TVHH; TVHH-EVL-EVH-EVL-EVH-TVHH; TVHH-EVH-EVL-EVH-EVL-TVHH; TVHH-EVL-EVH-EVL-EVH-TVHH-TVHH; TVHH-EVH-EVL-EVH-EVL-TVHH-TVHH; TVHH-TVHH-EVL-EVH-EVL-EVH-TVHH; TVHH-TVHH-EVH-EVL-EVH-EVL-TVHH; TVHH-EVL-EVH-EVL-EVH-TVHH-TVHH-TVHH; TVHH-EVH-EVL-EVH-EVL-TVHH-TVHH-TVHH; TVHH-TVHH-TVHH-EVL-EVH-EVL-EVH-TVHH; TVHH-TVHH-TVHH-EVH-EVL-EVH-EVL-TVHH; TVHH-TVHH-EVL-EVH-EVL-EVH-TVHH-TVHH; or TVHH-TVHH-EVH-EVL-EVH-EVL-TVHH-TVHH. In some embodiments, the linkers that connect the two effector cell antigen interacting domains (E) to each other comprise internal linkers. In some embodiments, the effector cell antigen interacting domain that is not connected directly to the one or more target cell antigen interacting domains comprises a terminal linker that connects EVL and EVH. In some embodiments, each of the internal linkers is 6-8 amino acids in length. In some embodiments, the terminal linker is 15 or 16 amino acids in length. In some embodiments, both of the effector cell antigen interacting domains are connected directly to target cell antigen interacting domains. In some embodiments, both of the effector cell antigen interacting domains are connected N-terminal and C-terminal to target cell antigen interacting domains. In some embodiments, both of the effector cell antigen interacting domains comprise a terminal linker that connects EVL and EVH. In some embodiments, the terminal linker is 15 or 16 amino acids in length. In some embodiments, the variable domains and the target cell antigen interacting domains are ordered according to the following N-terminal to C-terminal arrangements in which (-) is the linker: TVHH-EVH-EVL-TVHH-TVHH-EVH-EVL-TVHH; TVHH-EVL-EVH-TVHH-TVHH-EVH-EVL-TVHH; TVHH-EVH-EVL-TVHH-TVHH-EVL-EVH-TVHH; and TVHH-EVL-EVH-TVHH-TVHH-EVL-EVH-TVHH.

In some embodiments, each of the effector cell antigen interacting domains bind to an effector cell antigen. In some embodiments, the effector cell antigen comprises CD3, CD16a, or Death Receptor 5 (DR5). In some embodiments, the CD3 is CD3 delta, CD3 gamma, or CD3 epsilon. In some embodiments, the effector cell antigen is on an effector cell. In some embodiments, the effector cell comprises a T cell, NK cell, or a macrophage. In some embodiments, the target cell antigen interacting domains that interact with a target cell (T) bind to a target cell antigen. In some embodiments, the target cell antigen is clustered in a lipid raft when on a cell associated with a disease state and not clustered in a lipid raft when on a cell that is not associated with a disease state. In some embodiments, the target cell antigen is clustered when on a cell associated with a disease state and not clustered when on a cell that is not associated with a disease state. In some embodiments, the target cell antigen is a dimer, trimer, tetramer, or oligomer when on a cell associated with a disease state and a monomer or dimer when on a cell that is not associated with a disease state. In some embodiments, the target cell antigen is part of a cell signaling complex when on a cell associated with a disease state and not part of the same cell signaling complex when on a cell that is not associated with a disease state. In some embodiments, the target cell antigen is part of a cell signaling complex when on a cell associated with a disease state and not part of a cell signaling complex when on a cell that is not associated with a disease state. In some embodiments, the target cell antigen comprises CD33, FAP, EGFR, HER2, or EpCAM. In some embodiments, the cell associated with a disease state is a myeloid-cell, fibroblast, or cancer cell.

Disclosed herein are pharmaceutical compositions comprising: (a) the single chain polypeptide of any one of the above embodiments; and (b) a pharmaceutically acceptable excipient.

Discloses herein are isolated recombinant nucleic acids encoding a single chain polypeptide according any one of the above embodiments.

Discloses herein are vectors comprising an isolated nucleic acid according to the above embodiment.

Discloses herein are host cells comprising an isolated nucleic acid of the above embodiment or a vector according to the above embodiment.

Disclosed herein are methods of treating a cancer comprising administering to a subject in need thereof a single chain polypeptide according to any one of the above embodiments. In some embodiments, the cancer is a solid tumor cancer. In some embodiments, the cancer is a hematological cancer.

Disclosed herein are methods of treating an inflammatory disease or condition comprising administering to a subject in need thereof a single chain polypeptide according to any one of the above embodiments.

Disclosed herein are methods of treating an autoimmune disease or condition comprising administering to a subject in need thereof a single chain polypeptide according to any one of the above embodiments.

Disclosed herein are methods of treating a cardiovascular disease or condition comprising administering to a subject in need thereof a single chain polypeptide according to any one of the above embodiments.

Disclosed herein are methods of treating a fibrotic disease or condition comprising administering to a subject in need thereof a single chain polypeptide according to any one of the above embodiments.

Disclosed herein are methods of treating a bacterial infection comprising administering to the subject in need thereof a single chain polypeptide according to any one of the above embodiments.

Disclosed herein are methods of treating a viral infection comprising administering to the subject in need thereof a single chain polypeptide according to any one of the above embodiments.

Disclosed herein are single chain polypeptides comprising two variable domains of a single chain variable fragment (EV and EV) connected by a linker that fold into a conformation to form an effector cell antigen interacting domain (E) wherein the effector cell antigen interacting domain is connected by one or more linkers to two or more target cell antigen interacting domains that interacts with a target cell wherein the target cell antigen interacting domain is a VHH single domain antibody (TVHH), wherein the single chain polypeptide does not comprise an antibody constant domain. In some embodiments, the two variable domains of a single chain variable fragment (EV and EV) comprise a variable heavy chain of a single chain variable fragment (EVH) and a variable light chain of single chain variable fragment (EVL). In some embodiments, the two or more target cell antigen interacting domains are connected C-terminal to the effector cell antigen interacting domain. In some embodiments, the two or more target cell antigen interacting domains are connected N-terminal to the effector cell antigen interacting domain. In some embodiments, the two or more target cell antigen interacting domains are connected N-terminal and C-terminal to the effector cell antigen interacting domain. In some embodiments, the effector cell antigen interacting domain is connected to two target cell antigen interacting domains. In some embodiments, the effector cell antigen interacting domain is connected to three target cell antigen interacting domains. In some embodiments, the effector cell antigen interacting domain is connected to four target cell antigen interacting domains. In some embodiments, the effector cell antigen interacting domain is connected to five target cell antigen interacting domains. In some embodiments, the effector cell antigen interacting domain is connected to six target cell antigen interacting domains. In some embodiments, the effector cell antigen interacting domain is connected to two target cell antigen interacting domains and the variable domains and the two target cell antigen interacting domains are ordered according to the following N-terminal to C-terminal arrangements in which (-) is the linker: TVHH-TVHH-EVL-EVH; EVH-EVL-TVHH-TVHH; EVL-EVH-TVHH-TVHH; TVHH-TVHH-EVH-EVL; TVHH-EVL-EVH-TVHH; or TVHH-EVH-EVL-TVHH. In some embodiments, the effector cell antigen interacting domain is connected to three target cell antigen interacting domains and the variable domains and the three target cell antigen interacting domains are ordered according to the following N-terminal to C-terminal arrangements in which (-) is the linker: TVHH-TVHH-TVHH-EVL-EVH; EVH-EVL-TVHH-TVHH-TVHH; EVL-EVH-TVHH-TVHH-TVHH; TVHH-TVHH-TVHH-EVH-EVL; TVHH-TVHH-EVL-EVH-TVHH; TVHH-EVH-EVL-TVHH-TVHH; TVHH-EVL-EVH-TVHH-TVHH; or TVHH-TVHH-EVH-EVL-TVHH. In some embodiments, the effector cell antigen interacting domain is connected to four target cell antigen interacting domains and the variable domains and the four target cell antigen interacting domains are ordered according to the following N-terminal to C-terminal arrangements in which (-) is the linker: TVHH-TVHH-TVHH-TVHH-EVL-EVH; EVH-EVL-TVHH-TVHH-TVHH-TVHH; EVL-EVH-TVHH-TVHH-TVHH-TVHH; TVHH-TVHH-TVHH-TVHH-EVH-EVL; TVHH-TVHH-TVHH-EVL-EVH-TVHH; TVHH-EVH-EVL-TVHH-TVHH-TVHH; TVHH-EVL-EVH-TVHH-TVHH-TVHH; TVHH-TVHH-TVHH-EVH-EVL-TVHH; TVHH-TVHH-EVL-EVH-TVHH-TVHH; or TVHH-TVHH-EVH-EVL-TVHH-TVHH. In some embodiments, the effector cell antigen interacting domain is connected to five target cell antigen interacting domains and the variable domains and the five target cell antigen interacting domains are ordered according to the following N-terminal to C-terminal arrangements in which (-) is the linker: TVHH-TVHH-TVHH-TVHH-TVHH-EVL-EVH; EVH-EVL-TVHH-TVHH-TVHH-TVHH-TVHH; EVL-EVH-TVHH-TVHH-TVHH-TVHH-TVHH; TVHH-TVHH-TVHH-TVHH-TVHH-EVH-EVL; TVHH-TVHH-TVHH-TVHH-EVL-EVH-TVHH; TVHH-EVH-EVL-TVHH-TVHH-TVHH-TVHH; TVHH-EVL-EVH-TVHH-TVHH-TVHH-TVHH; TVHH-TVHH-TVHH-TVHH-EVH-EVL-TVHH; TVHH-TVHH-TVHH-EVL-EVH-TVHH-TVHH; TVHH-TVHH-EVH-EVL-TVHH-TVHH-TVHH; TVHH-TVHH-EVL-EVH-TVHH-TVHH-TVHH; or TVHH-TVHH-TVHH-EVH-EVL-TVHH-TVHH. In some embodiments, the effector cell antigen interacting domain is connected to six target cell antigen interacting domains and the variable domains and the six target cell antigen interacting domains are ordered according to the following N-terminal to C-terminal arrangements in which (-) is the linker: TVHH-TVHH-TVHH-TVHH-TVHH-TVHH-EVL-EVH; EVH-EVL-TVHH-TVHH-TVHH-TVHH-TVHH-TVHH; EVL-EVH-TVHH-TVHH-TVHH-TVHH-TVHH-TVHH; TVHH-TVHH-TVHH-TVHH-TVHH-TVHH-EVH-EVL; TVHH-TVHH-TVHH-TVHH-TVHH-EVL-EVH-TVHH; TVHH-EVH-EVL-TVHH-TVHH-TVHH-TVHH-TVHH; TVHH-EVL-EVH-TVHH-TVHH-TVHH-TVHH-TVHH; TVHH-TVHH-TVHH-TVHH-TVHH-EVH-EVL-TVHH; TVHH-TVHH-TVHH-TVHH-EVL-EVH-TVHH-TVHH; TVHH-TVHH-EVH-EVL-TVHH-TVHH-TVHH-TVHH; TVHH-TVHH-EVL-EVH-TVHH-TVHH-TVHH-TVHH; TVHH-TVHH-TVHH-TVHH-EVH-EVL-TVHH-TVHH; TVHH-TVHH-TVHH-EVL-EVH-TVHH-TVHH-TVHH; or TVHH-TVHH-TVHH-EVH-EVL-TVHH-TVHH-TVHH. In some embodiments, the effector cell antigen interacting domain is connected to six target cell antigen interacting domains and the variable domains and the six target cell antigen interacting domains are ordered according to the following N-terminal to C-terminal arrangements in which (-) is the linker: TVHH-TVHH-TVHH-EVH-EVL-TVHH-TVHH-TVHH; or EVH-EVL-TVHH-TVHH-TVHH-TVHH-TVHH-TVHH. In some embodiments, the effector cell antigen interacting domain comprises a terminal linker that connects EVL and EVH. In some embodiments, the terminal linker is 15 or 16 amino acids in length. In some embodiments, the one or more linkers that connects the two or more target cell antigen interacting domains comprises an internal linker. In some embodiments, each of the internal linkers is 6-8 amino acids in length. In some embodiments, the effector cell antigen interacting domains bind to an effector cell antigen. In some embodiments, the effector cell antigen comprises CD3, CD16a, or Death Receptor 5 (DR5). In some embodiments, the CD3 is CD3 delta, CD3 gamma, or CD3 epsilon. In some embodiments, the effector cell antigen is on an effector cell. In some embodiments, the effector cell comprises a T cell, NK cell, or a macrophage. In some embodiments, the target cell antigen interacting domain that interacts with a target cell (T) binds to a target cell antigen. In some embodiments, the target cell antigen is clustered in a lipid raft when on a cell associated with a disease state and not clustered in a lipid raft when on a cell that is not associated with a disease state. In some embodiments, the target cell antigen is clustered when on a cell associated with a disease state and not clustered when on a cell that is not associated with a disease state. In some embodiments, the target cell antigen is a dimer, trimer, tetramer, or oligomer when on a cell associated with a disease state and a monomer or dimer when on a cell that is not associated with a disease state. In some embodiments, the target cell antigen is part of a cell signaling complex when on a cell associated with a disease state and not part of the same cell signaling complex when on a cell that is not associated with a disease state. In some embodiments, the target cell antigen is part of a cell signaling complex when on a cell associated with a disease state and not part of a cell signaling complex when on a cell that is not associated with a disease state. In some embodiments, the target cell antigen comprises CD33, FAP, EGFR, HER2, or EpCAM. In some embodiments, the cell associated with a disease state is a myeloid-cell, fibroblast, or cancer cell. In some embodiments, the single chain polypeptide has one effector cell antigen interacting domain.

Disclosed herein are pharmaceutical compositions comprising: (a) the single chain polypeptide according to any of the above embodiments; and (b) a pharmaceutically acceptable excipient.

Disclosed herein are isolated recombinant nucleic acid encoding a single chain polypeptide according any one of the above embodiments.

Disclosed herein are vectors comprising an isolated nucleic acid according to the preceding embodiment.

Disclosed here are host cells comprising an isolated nucleic acid according to any of the preceding embodiment or a vector according to the preceding embodiment.

Disclosed here are methods of treating a cancer comprising administering to a subject in need thereof a single chain polypeptide according to any one of the preceding embodiments. In some embodiments, the cancer is a solid tumor cancer. In some embodiments, the cancer is a hematological cancer. In some embodiments, are methods of treating an inflammatory disease or condition comprising administering to a subject in need thereof a single chain polypeptide according to any one of the preceding embodiments. Disclosed here are methods of treating an autoimmune disease or condition comprising administering to a subject in need thereof a single chain polypeptide according to any one of the preceding embodiments. Disclosed here are methods of treating a cardiovascular disease or condition comprising administering to a subject in need thereof a single chain polypeptide according to any one of the preceding embodiments. Disclosed herein are methods of treating a fibrotic disease or condition comprising administering to a subject in need thereof a single chain polypeptide according to any one of the preceding embodiments. Disclosed herein are methods of treating a bacterial infection comprising administering to the subject in need thereof a single chain polypeptide according to any one of the preceding embodiments. Disclosed herein are methods of treating a viral infection comprising administering to the subject in need thereof a single chain polypeptide according to any one of the preceding embodiments.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B illustrate certain non-limiting embodiments of the single chain polypeptides of the current disclosure in which the single chain polypeptide comprises variable domains connected by linkers that fold into a conformation such that the variable domains form two effector cell antigen interacting domains (E) wherein each effector cell antigen interacting domain comprises two variable domains of a single chain variable fragment (EV and EV), and one of the effector cell antigen interacting domains is linked to two target cell antigen interacting domains that interact with a target cell wherein the target cell antigen interacting domain is a VHH single domain antibody (TVHH). The variable domains and the target cell antigen interacting domains are ordered according to the following N-terminal to C-terminal arrangements in which (-) is a linker: FIG. 1A EVL-EVH-EVL-EVH-TVHH-TVHH; and FIG. 1B EVH-EVL-EVH-EVL-TVHH-TVHH.

FIG. 2A and FIG. 2B illustrate certain non-limiting embodiments of the single chain polypeptides of the current disclosure in which the single chain polypeptide comprises variable domains connected by linkers that fold into a conformation such that the variable domains form two effector cell antigen interacting domains (E) wherein each effector cell antigen interacting domain comprises two variable domains of a single chain variable fragment (EVL and EVH), and one of the effector cell antigen interacting domains is linked to four target cell antigen interacting domains that interacts with a target cell wherein the target cell antigen interacting domain is a VHH single domain antibody (TVHH). The variable domains and the target cell antigen interacting domains are ordered according to the following N-terminal to C-terminal arrangements in which (-) is a linker: FIG. 2A EVL-EVH-EVL-EVH-TVHH-TVHH-TVHH-TVHH; and FIG. 2B EVH-EVL-EVH-EVL-TVHH-TVHH-TVHH-TVHH.

FIG. 3A and FIG. 3B illustrate certain non-limiting embodiments of the single chain polypeptides of the current disclosure in which the single chain polypeptide comprises variable domains connected by linkers that fold into a conformation such that the variable domains form two effector cell antigen interacting domains (E) wherein each effector cell antigen interacting domain comprises two variable domains of a single chain variable fragment (EVL and EVH), and one of the effector cell antigen interacting domains is linked to four target cell antigen interacting domains that interacts with a target cell wherein the target cell antigen interacting domain is a VHH single domain antibody (TVHH). The variable domains and the target cell antigen interacting domains are ordered according to the following N-terminal to C-terminal arrangements in which (-) is a linker: FIG. 3A TVHH-TVHH-EVL-EVH-EVL-EVH-TVHH-TVHH; and FIG. 3B TVHH-TVHH-EVH-EVL-EVH-EVL-TVHH-TVHH.

FIG. 4A and FIG. 4B illustrate certain non-limiting embodiments of the single chain polypeptides of the current disclosure in which the single chain polypeptide comprises variable domains connected by linkers that fold into a conformation such that the variable domains form two effector cell antigen interacting domains (E) wherein each effector cell antigen interacting domain comprises two variable domains of a single chain variable fragment (EV and EV), and one of the effector cell antigen interacting domains is linked to one target cell antigen interacting domains that interact with a target cell wherein the target cell antigen interacting domain is a VHH single domain antibody (TVHH). The variable domains and the target cell antigen interacting domains are ordered according to the following N-terminal to C-terminal arrangements in which (-) is a linker: FIG. 4A EVL-EVH-EVL-EVH-TVHH; and FIG. 4B EVH-EVL-EVH-EVL-TVHH.

FIG. 5A and FIG. 5B illustrate certain non-limiting embodiments of the single chain polypeptides of the current disclosure in which the single chain polypeptide comprises variable domains connected by linkers that fold into a conformation such that the variable domains form two effector cell antigen interacting domains (E) wherein each effector cell antigen interacting domain comprises two variable domains of a single chain variable fragment (EVL and EVH), and one of the effector cell antigen interacting domains is linked to three target cell antigen interacting domains that interacts with a target cell wherein the target cell antigen interacting domain is a VHH single domain antibody (TVHH). The variable domains and the target cell antigen interacting domains are ordered according to the following N-terminal to C-terminal arrangements in which (-) is a linker: FIG. 5A EVL-EVH-EVL-EVH-TVHH-TVHH-TVHH; and FIG. 5B EVH-EVL-EVH-EVL-TVHH-TVHH-TVHH.

FIG. 6A and FIG. 6B illustrate certain non-limiting embodiments of the single chain polypeptides of the current disclosure in which the single chain polypeptide comprises variable domains connected by linkers that fold into a conformation such that the variable domains form two effector cell antigen interacting domains (E) wherein each effector cell antigen interacting domain comprises two variable domains of a single chain variable fragment (EVL and EVH), and one of the effector cell antigen interacting domains is linked to two target cell antigen interacting domains that interact with a target cell wherein the target cell antigen interacting domain is a VHH single domain antibody (TVHH). The variable domains and the target cell antigen interacting domains are ordered according to the following N-terminal to C-terminal arrangements in which (-) is a linker: FIG. 6A TVHH-EVL-EVH-EVL-EVH-TVHH; and FIG. 6B TVHH-EVH-EVL-EVH-EVL-TVHH.

FIG. 7A and FIG. 7B illustrate T cell killing activity in a cytotoxicity assay on MCF7 cancer cells by exemplary single chain polypeptide constructs.

FIG. 8A and FIG. 8B illustrate T cell killing activity in a cytotoxicity assay on SKBR3 cancer cells by exemplary single chain polypeptide constructs.

FIG. 9 illustrates T cell activation in MCF7 cancer cells by exemplary single chain polypeptide constructs.

FIG. 10 illustrates T cell activation in SKBR3 cancer cells by exemplary single chain polypeptide constructs.

### DETAILED DESCRIPTION OF THE INVENTION

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

Disclosed herein are single chain polypeptides comprising variable domains connected by linkers that fold into a conformation such that the variable domains form two effector cell antigen interacting domains (E) wherein each effector cell antigen interacting domain comprises two variable domains of a single chain variable fragment (EV and EV), and at least one of the effector cell antigen interacting domains is linked to one or more target interacting domains. In some embodiments, the target interacting domain binds to a tumor antigen, a ligand, a protein, a lipid, nucleic acid, or a polysaccharide. In some embodiments, the tumor antigen is on a cell in a tumor microenvironment. In some embodiments, the target interacting domain comprises an antibody, an antibody fragment, a ligand, or a cofactor. In some embodiments, the antibody or antibody fragment of the target interacting domain comprises a Fab, a Fab′, a F(ab′)₂, a Fv fragment, a single-chain Fv (scFv), a tandem single-chain Fv ((scFv)₂, a dual affinity retargeting antibody, a diabody, a VHH single domain antibody, a TriTac, an Adnectin, an Anticalin, an Avimer, a Fynomer, a Kunitz domain, a knottin, an Affibody, or a DARPin.

In some embodiments, the one or more target interacting domain comprises one or more target cell antigen interacting domain that binds to a target cell antigen. In some embodiments, the target cell antigen interacting domains binds to more than one target cell antigen. In some embodiments, the target cell antigen interacting domain is bispecific or multispecific. In some embodiments, the target cell antigen interacting domain comprises a VHH single domain antibody (TVHH). In some embodiments, the single chain polypeptide does not contain a fragment crystallizable (Fc) domain.

Described herein are single chain polypeptides comprising variable domains connected by linkers that fold into a conformation such that the variable domains form two effector cell antigen interacting domains (E) wherein each effector cell antigen interacting domain comprises two variable domains of a single chain variable fragment (EV and EV), and one of the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain.

### Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The terms “polypeptide” and “protein” are used interchangeably to refer to a polymer of amino acid residues, and are not limited to a minimum length. Polypeptides, including the provided polypeptides and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, a “antigen binding domain” or “antigen interacting domain” refers to an immunoglobulin derivative with antigen binding properties, i.e. immunoglobulin polypeptides or fragments thereof that contain an antigen binding site. In some cases, the binding domain comprises a variable domain of an antibody or fragments thereof. Each antigen-binding domain is formed by an antibody, i.e. immunoglobulin, variable heavy chain domain (VH) and an antibody variable light chain domain (VL) binding to the same epitope, whereas the variable heavy chain domain (VH) comprises three heavy chain complementarity determining regions (CDR): CDR1, CDR2 and CDR3; and the variable light chain domain (VL) comprises three light chain complementarity determining regions (CDR): CDR1, CDR2 and CDR3. In some instances, the binding domain according to some embodiments herein is devoid of immunoglobulin constant domains. In some instances, the variable light and heavy chain domains forming the antigen binding site is covalently linked with one another, e.g. by a peptide linker, or in other instances, the variable light and heavy chain domains non-covalently associate with one another to form the antigen binding site. Binding domains and interacting domains also refer to antibody fragments or antibody derivatives including, for example, Fab, Fab′, F(ab′)2, Fv fragments, single-chain Fv, tandem single-chain Fv ((scFv)2, Bi-specific T-cell engagers (BiTE®), dual affinity retargeting antibodies (DART™), diabody, DuoBody® IgG molecules, single domain antibodies (e.g., VHH), TriTacs, and the like. Furthermore, in certain instances, the binding domain is multivalent, i.e. has two, three or more binding sites for one or more antigens.

In certain embodiments, the antigen binding or antigen interacting domains comprise sequences derived from the complementarity determining regions of antibodies. The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plickthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

In certain embodiments, the antigen binding or antigen interacting domains comprise sequences derived from the complementarity determining regions of antibodies. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

In certain embodiments, the antigen binding or antigen interacting domains are “humanized.” A “humanized” polypeptide or antibody is is one in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided polypeptides and antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

The polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the polypeptides described herein can be used to infect, transfect, transform, or otherwise render a suitable cell transgenic for the nucleic acid, thus enabling the production of polypeptide for commercial or therapeutic uses. Standard cell lines and methods for the production of antibodies or polypeptides from a large-scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing polypeptides or antibodies is a Chines Hamster Ovary cell (CHO) cell, an NSO murine myeloma cell, or a PER.C6® cell. In certain embodiments, the nucleic acid encoding the antibody is integrated into a genomic locus of a cell useful for producing polypeptides or antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody or polypeptide described herein integrated at a genomic location; and (b) a cryoprotectant. In certain embodiments, the cryoprotectant comprises glycerol or DMSO. In certain embodiments, the master cell bank is contained in a suitable vial or container able to withstand freezing by, and storage at liquid nitrogen or temperatures equivalent thereto.

Also described herein are methods of making a polypeptide described herein. Such methods comprise incubating a cell or cell-line comprising a nucleic acid encoding the polypeptide in a cell culture medium under conditions sufficient to allow for expression and secretion of the antibody, and further harvesting the polypeptide from the cell culture medium. The harvesting can further comprise one or more purification steps to remove live cells, cellular debris, non-target proteins or polypeptides, undesired salts, buffers, and medium components. In certain embodiments, the additional purification step(s) include centrifugation, ultracentrifugation, protein A, protein G, protein A/G, or protein L purification, size-exclusion chromatography, hydrophobic interaction chromatography, and/or ion exchange chromatography.

### Single Chain Polveptide Compositions

Disclosed herein are single chain polypeptides that fold to form a three-dimensional binding structure. The single chain polypeptides comprise antigen binding domains capable of binding to an effector cell antigen and a target cell antigen. In certain embodiments, single chain polypeptides are bispecific and bivalent, in other cases bispecific and trivalent (e.g., comprising three antigen binding domains). In certain embodiments, the single chain polypeptides are trivalent with respect to the effector cell antigen or the target cell antigen. In certain embodiments, the single chain polypeptides are bispecific and bivalent in other cases bispecific and quadrivalent (e.g., comprising four antigen binding domains). In certain embodiments, single chain polypeptides may be quadrivalent with respect to the effector cell antigen or the target cell antigen. In certain embodiments, the target cell antigen is a tumor associated antigen (e.g., CD33, FAP, EGFR, HER2 or EpCAM). In certain embodiments, the effector cell antigen is an effector cell associated antigen that is expressed by an immune cell (e.g., a T cell, NK cell, or NKT cell). Certain non-limiting embodiments of the single chain polypeptides are discussed in the following paragraphs.

Described herein in certain embodiments are single chain polypeptides comprising variable domains connected by linkers that fold into a conformation such that the variable domains form one or more effector cell antigen interacting domains (E) wherein the effector cell antigen interacting domain comprises two variable domains of a single chain variable fragment (EV and EV), and the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain.

Described herein in certain embodiments are single chain polypeptides comprising variable domains connected by linkers that fold into a conformation such that the variable domains form two effector cell antigen interacting domains (E) wherein each effector cell antigen interacting domain comprises two variable domains of a single chain variable fragment (EV and EV), and one of the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain.

In certain embodiments, the two variable domains of a single chain variable fragment (EV and EV) comprise a variable heavy chain of a single chain variable fragment (EVH) and a variable light chain of single chain variable fragment (EVL).

### Branched Three Dimensional Configuration of Target Cell Antigen Interacting Domains

In some embodiments, the EVL of the one of the effector cell antigen interacting domains that is linked to the one or more target cell antigen interacting domains is linked to at least one TVHH, and the EVH of the one of the effector cell antigen interacting domains that is linked to the one or more target cell antigen interacting domains is linked to at least one TVHH. In some embodiments, the at least one TVHH that is linked to the EVL binds to the same target antigen as the at least one TVHH that is linked to the EVH. In some embodiments, the at least one TVHH that is linked to the EVL binds to the same target antigen and has the same complementarity determining region (CDR) sequences as the at least one TVHH that is linked to the EVH. In some embodiments, the at least one TVHH that is linked to the EVL binds to the same target antigen and have no complementarity determining region (CDR) sequences in common as the at least one TVHH that is linked to the EVH. In some embodiments, the at least one TVHH that is linked to the EVL binds to a different target antigen as compared to the at least one TVHH that is linked to the EVH.

In some embodiments, the EVL and the EVH are linked to the same number of TVHHs. In some embodiments, the EVL is linked to a different number of TVHHs as compared to the number of TVHHs that are linked to EVH.

In some embodiments, the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to one TVHH.

In some embodiments, the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to two TVHH.

In some embodiments, the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to three TVHH.

In some embodiments, the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to two TVHH.

In some embodiments, the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to three TVHH.

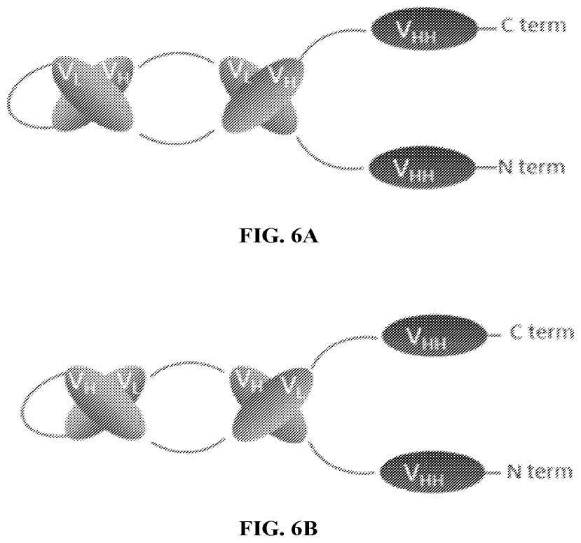

In some embodiments, the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to one TVHH. FIGS. 6A and 6B exemplify single chain polypeptides in which the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to one TVHH.

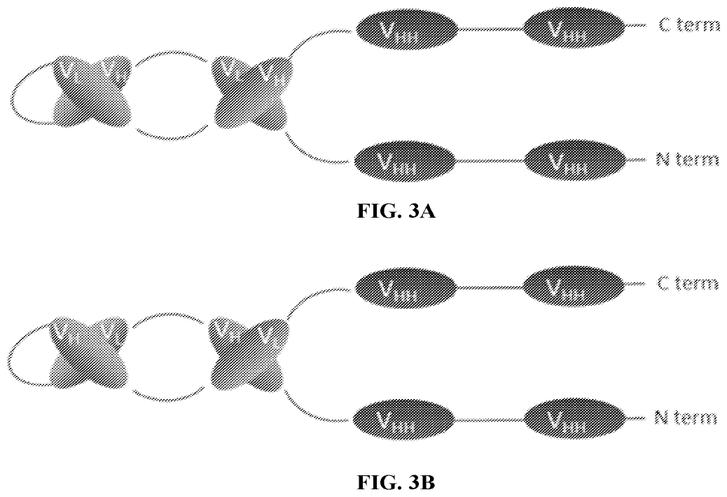

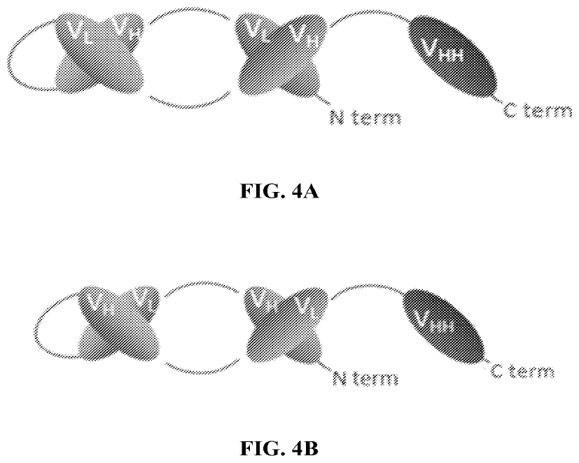

In some embodiments, the EVH of the one of the effector cell antigen interacting domains that is connected to the one or more target cell antigen interacting domains is connected to two TVHH, and the EVL of the one of the effector cell antigen interacting domains that is connected to the one or more target cell antigen interacting domains is connected to two TVHH. FIGS. 3A and 3B exemplify single chain polypeptides in which the EVH of the one of the effector cell antigen interacting domains that is connected to the one or more target cell antigen interacting domains is connected to two TVHH, and the EVL of the one of the effector cell antigen interacting domains that is connected to the one or more target cell antigen interacting domains is connected to two TVHH.

In some embodiments, the EVH of the one of the effector cell antigen interacting domains that is linked to the one or more target cell antigen interacting domains is linked to three TVHH, and the EVL of the one of the effector cell antigen interacting domains that is linked to the one or more target cell antigen interacting domains is linked to three TVHH.

In some embodiments, the EVH of the one of the effector cell antigen interacting domains that is linked to the one or more target cell antigen interacting domains is linked to four TVHH, and the EVL of the one of the effector cell antigen interacting domains that is linked to the one or more target cell antigen interacting domains is linked to four TVHH.

In some embodiments, the EVH of the one of the effector cell antigen interacting domains that is linked to the one or more target cell antigen interacting domains is linked to five TVHH, and the EVL of the one of the effector cell antigen interacting domains that is linked to the one or more target cell antigen interacting domains is linked to five TVHH.

In some embodiments, the EVH of the one of the effector cell antigen interacting domains that is linked to the one or more target cell antigen interacting domains is linked to six TVHH, and the EVL of the one of the effector cell antigen interacting domains that is linked to the one or more target cell antigen interacting domains is linked to six TVHH.

In some embodiments, the EVH of the one of the effector cell antigen interacting domains that is linked to the one or more target cell antigen interacting domains is linked to seven TVHH, and the EVL of the one of the effector cell antigen interacting domains that is linked to the one or more target cell antigen interacting domains is linked to seven TVHH.

In some embodiments, the EVH of the one of the effector cell antigen interacting domains that is linked to the one or more target cell antigen interacting domains is linked to eight TVHH, and the EVL of the one of the effector cell antigen interacting domains that is linked to the one or more target cell antigen interacting domains is linked to eight TVHH.

### Linear Three Dimensional Configuration of Target Cell Antigen Interacting Domains

In some embodiments, the one of the effector cell antigen interacting domains that is linked to the one or more target cell antigen interacting domains is connected to one target cell antigen interacting domain. In some embodiments, the one target cell antigen interacting domain is connected C-terminal to the one of the effector cell antigen interacting domains. In some embodiments, the one target cell antigen interacting domains is connected N-terminal to the one of the effector cell antigen interacting domains. FIGS. 4A and 4B exemplify single chain polypeptides in which one target cell antigen interacting domains is connected C-terminal to the one of the effector cell antigen interacting domains.

In some embodiments, the at least one of the effector cell antigen interacting domains that is linked to the one or more target cell antigen interacting domains is linked to more than one target cell antigen interacting domain. In some embodiments, all of the more than one target cell antigen interacting domains bind to the same target antigen. In some embodiments, all of the more than one target cell antigen interacting domains bind to the same target antigen and all have the same CDR sequences. In some embodiments, all of the more than one target cell antigen interacting domains bind to the same target antigen and at least two of the more than one target cell antigen interacting domains have no CDR sequences in common. In some embodiments, at least two of the more than one target cell antigen interacting domains bind to different target antigens.

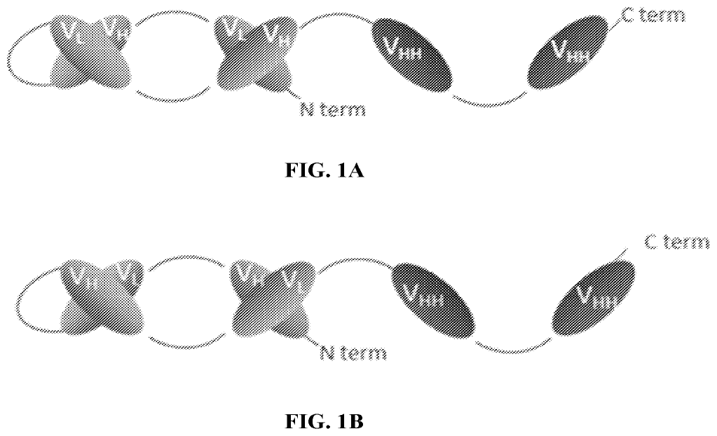

In some embodiments, the one of the effector cell antigen interacting domains that is linked to the one or more target cell antigen interacting domains is connected to two target cell antigen interacting domains. In some embodiments, the two-target cell antigen interacting domains are connected C-terminal to the one of the effector cell antigen interacting domains. FIGS. 1A and 1B exemplify single chain polypeptides in which two target cell antigen interacting domains are connected C-terminal to the one of the effector cell antigen interacting domains.

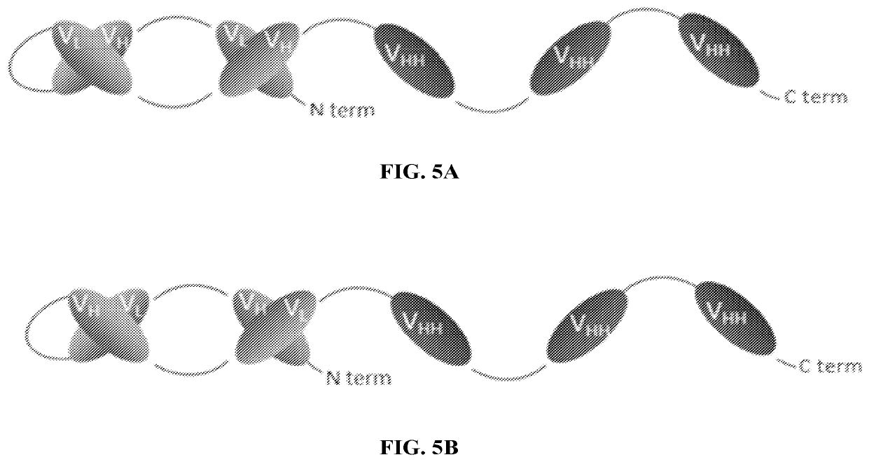

In some embodiments, the one of the effector cell antigen interacting domains that is linked to the one or more target cell antigen interacting domains is connected to three target cell antigen interacting domains. In some embodiments, the three-target cell antigen interacting domains are connected C-terminal to the one of the effector cell antigen interacting domains. In some embodiments, the three-target cell antigen interacting domains are connected N-terminal to the one of the effector cell antigen interacting domains. FIGS. 5A and 5B exemplify single chain polypeptides in which three target cell antigen interacting domains are connected C-terminal to the one of the effector cell antigen interacting domains.

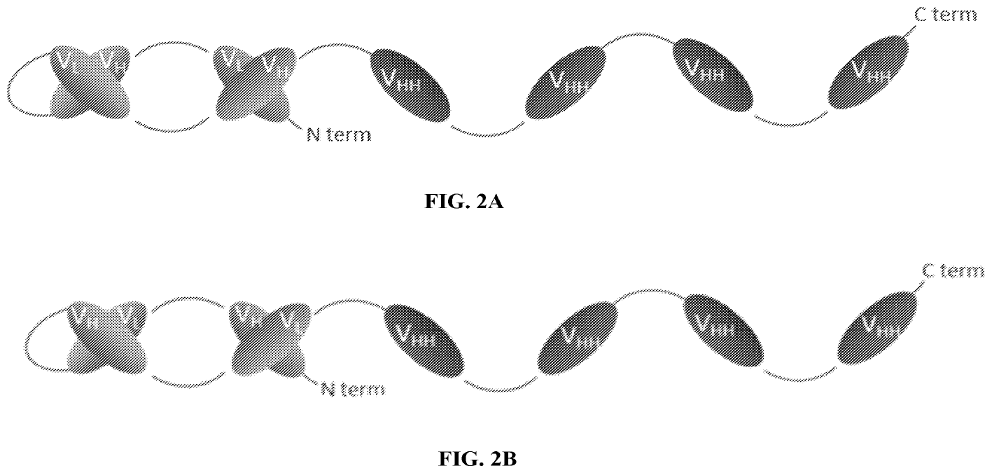

In some embodiments, the one of the effector cell antigen interacting domains that is linked to the one or more target cell antigen interacting domains is connected to four target cell antigen interacting domains. In some embodiments, the four-target cell antigen interacting domains are connected C-terminal to the one of the effector cell antigen interacting domains. FIGS. 2A and 2B exemplify single chain polypeptides in which four target cell antigen interacting domains are connected C-terminal to the one of the effector cell antigen interacting domains.

### N-Terminal to C-Terminal Arrangements of Single Chain Polypeptides

Further configurations and specific embodiments for the arrangement of target cell antigen interacting domains (T) or effector cell antigen interacting domains (E) are listed below. In certain embodiments, the single chain polypeptide comprises one or more target cell antigen interacting domains (T) and one or more effector cell antigen interacting domains (E), wherein the effector cell antigen interacting domains and the one or more target cell antigen interacting domains are ordered according to any one or more of the following N-terminal to C-terminal arrangements in which (-) is the linker: TVHH-EV-EV-EV-EV; EV-EV-EV-EV-TVHH; TVHH-TVHH-EV-EV-EV-EV; EV-EV-EV-EV-TVHH-TVHH; TVHH-TVHH-TVHH-EV-EV-EV-EV; EV-EV-EV-EV-TVHH-TVHH-TVHH; TVHH-TVHH-TVHH-TVHH-EV-EV-EV-EV; EV-EV-EV-EV-TVHH-TVHH-TVHH-TVHH; TVHH-EV-EV-EV-EV-TVHH; TVHH-EV-EV-EV-EV-TVHH-TVHH; TVHH-TVHH-EV-EV-EV-EV-TVHH; TVHH-EV-EV-EV-EV-TVHH-TVHH-TVHH; TVHH-TVHH-TVHH-EV-EV-EV-EV-TVHH or TVHH-TVHH-EV-EV-EV-EV-TVHH-TVHH.

In some embodiments, the variable domains and the one or more target cell antigen interacting domains are ordered according to the following N-terminal to C-terminal arrangements in which (-) is the linker: TVHH-EV-EV-EV-EV; EV-EV-EV-EV-TVHH; TVHH-TVHH-EV-EV-EV-EV; EV-EV-EV-EV-TVHH-TVHH; TVHH-TVHH-TVHH-EV-EV-EV-EV; EV-EV-EV-EV-TVHH-TVHH-TVHH; TVHH-TVHH-TVHH-TVHH-EV-EV-EV-EV; EV-EV-EV-EV-TVHH-TVHH-TVHH-TVHH; TVHH-EV-EV-EV-EV-TVHH; TVHH-EV-EV-EV-EV-TVHH-TVHH; TVHH-TVHH-EV-EV-EV-EV-TVHH; TVHH-EV-EV-EV-EV-TVHH-TVHH-TVHH; TVHH-TVHH-TVHH-EV-EV-EV-EV-TVHH; TVHH-TVHH-EV-EV-EV-EV-TVHH-TVHH; TVHH-TVHH-TVHH-EV-EV-EV-EV-TVHH-TVHH-TVHH; TVHH-TVHH -TVHH-TVHH-EV-EV-EV-EV-TVHH-TVHH-TVHH-TVHH; TVHH-TVHH-TVHH-TVHH-TVHH-EV-EV-EV-EV-TVHH-TVHH-TVHH-TVHH-TVHH; TVHH-TVHH-TVHH-TVHH-TVHH-TVHH-EV-EV-EV-EV-TVHH-TVHH-TVHH-TVHH-TVHH-TVHH; TVHH-TVHH-TVHH-TVHH-TVHH-TVHH-TVHH-EV-EV-EV-EV-TVHH-TVHH-TVHH-TVHH-TVHH-TVHH-TVHH; or TVHH-TVHH-TVHH-TVHH-TVHH-TVHH-TVHH-TVHH-EV-EV-EV-EV-TVHH-TVHH-TVHH-TVHH-TVHH-TVHH-TVHH-TVHH.

In some embodiments, the variable domains and the one or more target cell antigen interacting domains are ordered according to the following N-terminal to C-terminal arrangements in which (-) is the linker: TVHH-TVHH-EV-EV-EV-EV-TVHH-TVHH; or TVHH-EV-EV-EV-EV-TVHH. In some embodiments, the variable domains and the one or more target cell antigen interacting domains are ordered according to the following N-terminal to C-terminal arrangements in which (-) is the linker: TVHH-TVHH-EV-EV-EV-EV-TVHH-TVHH. In some embodiments, the variable domains and the one or more target cell antigen interacting domains are ordered according to the following N-terminal to C-terminal arrangements in which (-) is the linker: TVHH-EV-EV-EV-EV-TVHH.

In some embodiments, the variable domains and the one or more target cell antigen interacting domains are ordered according to the following N-terminal to C-terminal arrangements in which (-) is the linker: TVHH-TVHH-EV-EV-EV-EV-TVHH-TVHH.

In some embodiments, the variable domains and the one or more target cell antigen interacting domains are ordered according to the following N-terminal to C-terminal arrangements in which (-) is the linker: TVHH-EV-EV-EV-EV-TVHH.

In certain embodiments, the single chain polypeptide comprises one or more target cell antigen interacting domains (T) and one or more effector cell antigen interacting domains (E), wherein the one or more effector cell antigen interacting domains and the one or more target cell antigen interacting domains are ordered according to one or more of the following N-terminal to C-terminal arrangements in which (-) is the linker: TVHH-EVL-EVH-EVL-EVH; TVHH-EVH-EVL-EVH-EVL; EVL-EVH-EVL-EVH-TVHH; EVH-EVL-EVH-EVL-TVHH; TVHH-TVHH-EVL-EVH-EVL-EVH; TVHH-TVHH-EVH-EVL-EVH-EVL; EVL-EVH-EVL-EVH-TVHH-TVHH; EVH-EVL-EVH-EVL-TVHH-TVHH; TVHH-TVHH-TVHH-EVL-EVH-EVL-EVH; TVHH-TVHH-TVHH-EVH-EVL-EVH-EVL; EVL-EVH-EVL-EVH-TVHH-TVHH-TVHH; EVH-EVL-EVH-EVL-TVHH-TVHH-TVHH; TVHH-TVHH-TVHH-TVHH-EVL-EVH-EVL-EVH; TVHH-TVHH-TVHH-TVHH-EVH-EVL-EVH-EVL; EVL-EVH-EVL-EVH-TVHH-TVHH-TVHH-TVHH; EVH-EVL-EVH-EVL-TVHH-TVHH-TVHH-TVHH; TVHH-EVL-EVH-EVL-EVH-TVHH; TVHH-EVH-EVL-EVH-EVL-TVHH; TVHH-EVL-EVH-EVL-EVH-TVHH-TVHH; TVHH-EVH-EVL-EVH-EVL-TVHH-TVHH; TVHH-TVHH-EVL-EVH-EVL-EVH-TVHH; TVHH-TVHH-EVH-EVL-EVH-EVL-TVHH; TVHH-EVL-EVH-EVL-EVH-TVHH-TVHH-TVHH; TVHH-EVH-EVL-EVH-EVL-TVHH-TVHH-TVHH; TVHH-TVHH-TVHH-EVL-EVH-EVL-EVH-TVHH; TVHH-TVHH-TVHH-EVH-EVL-EVH-EVL-TVHH; TVHH-TVHH-EVL-EVH-EVL-EVH-TVHH-TVHH; or TVHH-TVHH-EVH-EVL-EVH-EVL-TVHH-TVHH.

In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH; TVHH-TVHH-EVH-EVL-EVH-EVL-TVHH-TVHH; TVHH-EVL-EVH-EVL-EVH-TVHH; or TVHH-EVH-EVL-EVH-EVL-TVHH. In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH; or TVHH-TVHH-EVH-EVL-EVH-EVL-TVHH-TVHH. In some embodiments, the variable domains and the one or more target cell antigen interacting domains are ordered according to the following N-terminal to C-terminal arrangements in which (-) is the linker: TVHH-EVL-EVH-EVL-EVH-TVHH; or TVHH-EVH-EVL-EVH-EVL-TVHH. In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH. In some embodiments, the variable domains and the one or more target cell antigen interacting domains are ordered according to the following N-terminal to C-terminal arrangements in which (-) is the linker: TVHH-TVHH-EVH-EVL-EVH-EVL-TVHH-TVHH. In some embodiments, the variable domains and the one or more target cell antigen interacting domains are ordered according to the following N-terminal to C-terminal arrangements in which (-) is the linker: TVHH-EVL-EVH-EVL-EVH-TVHH. In some embodiments, the variable domains and the one or more target cell antigen interacting domains are ordered according to the following N-terminal to C-terminal arrangements in which (-) is the linker: TVHH-EVH-EVL-EVH-EVL-TVHH.

In some embodiments, the variable domains and the one or more target cell antigen interacting domains are ordered according to the following N-terminal to C-terminal arrangements in which (-) is the linker: TVHH-TVHH-EVL-EVH-EVL-EVH-TVHH-TVHH.

In some embodiments, the variable domains and the one or more target cell antigen interacting domains are ordered according to the following N-terminal to C-terminal arrangements in which (-) is the linker: TVHH-TVHH-EVH-EVL-EVH-EVL-TVHH-TVHH.

In some embodiments, the variable domains and the one or more target cell antigen interacting domains are ordered according to the following N-terminal to C-terminal arrangements in which (-) is the linker: TVHH-EVL-EVH-EVL-EVH-TVHH.

In some embodiments, the variable domains and the one or more target cell antigen interacting domains are ordered according to the following N-terminal to C-terminal arrangements in which (-) is the linker: TVHH-EVH-EVL-EVH-EVL-TVHH.

In some embodiments, the variable domains and the target cell antigen interacting domains are ordered according to the following N-terminal to C-terminal arrangements in which (-) is the linker: TVHH-EVH-EVL-TVHH-TVHH-EVH-EVL-TVHH; TVHH-EVL-EVH-TVHH-TVHH-EVH-EVL-TVHH; TVHH-EVH-EVL-TVHH-TVHH-EVL-EVH-TVHH; and TVHH-EVL-EVH-TVHH-TVHH-EVL-EVH-TVHH.

In some embodiments, the variable domains and the target cell antigen interacting domains are ordered according to the following N-terminal to C-terminal arrangements in which (-) is the linker: TVHH-EVH-EVL-TVHH-TVHH-EVH-EVL-TVHH.

In some embodiments, the variable domains and the target cell antigen interacting domains are ordered according to the following N-terminal to C-terminal arrangements in which (-) is the linker: TVHH-EVL-EVH-TVHH-TVHH-EVH-EVL-TVHH.

In some embodiments, the variable domains and the target cell antigen interacting domains are ordered according to the following N-terminal to C-terminal arrangements in which (-) is the linker: TVHH-EVH-EVL-TVHH-TVHH-EVL-EVH-TVHH.

In some embodiments, the variable domains and the target cell antigen interacting domains are ordered according to the following N-terminal to C-terminal arrangements in which (-) is the linker: TVHH-EVL-EVH-TVHH-TVHH-EVL-EVH-TVHH.

### Additional Configurations

Disclosed herein are single chain polypeptides comprising two variable domains of a single chain variable fragment (EV and EV) connected by a linker that fold into a conformation to form an effector cell antigen interacting domain (E) wherein the effector cell antigen interacting domain is connected by one or more linkers to two or more target cell antigen interacting domains that interacts with a target cell wherein the target cell antigen interacting domain is a VHH single domain antibody (TVHH), wherein the single chain polypeptide does not comprise an antibody constant domain.

In some embodiments, the two variable domains of a single chain variable fragment (EV and EV) comprise a variable heavy chain of a single chain variable fragment (EVH) and a variable light chain of single chain variable fragment (EVL).

In some embodiments, the two or more target cell antigen interacting domains are connected C-terminal to the effector cell antigen interacting domain. FIG. 4B exemplifies a single chain polypeptide in which six target cell antigen interacting domains are connected C-terminal to the effector cell antigen interacting domain. In some embodiments, the two or more target cell antigen interacting domains are connected N-terminal to the effector cell antigen interacting domain. In some embodiments, the two or more target cell antigen interacting domains are connected N-terminal and C-terminal to the effector cell antigen interacting domain. FIG. 4A exemplifies a single chain polypeptide in which six target cell antigen interacting domains are connected N-terminal and C-terminal to the effector cell antigen interacting domain.

In some embodiments, the effector cell antigen interacting domain is connected to two target cell antigen interacting domains. In some embodiments, the effector cell antigen interacting domain is connected to three target cell antigen interacting domains. In some embodiments, the effector cell antigen interacting domain is connected to four target cell antigen interacting domains. In some embodiments, the effector cell antigen interacting domain is connected to five target cell antigen interacting domains. In some embodiments, the effector cell antigen interacting domain is connected to six target cell antigen interacting domains. In some embodiments, the effector cell antigen interacting domain is connected to seven target cell antigen interacting domains. In some embodiments, the effector cell antigen interacting domain is connected to eight target cell antigen interacting domains. In some embodiments, the effector cell antigen interacting domain is connected to nine target cell antigen interacting domains. In some embodiments, the effector cell antigen interacting domain is connected to ten target cell antigen interacting domains.

### N-Terminal to C-Terminal Arrangements of Single Chain Polypeptides

Further configurations and specific embodiments for the arrangement of target cell antigen interacting domains (T) or effector cell antigen interacting domains (E) are listed below.

In some embodiments, the effector cell antigen interacting domain is connected to two target cell antigen interacting domains and the variable domains and the two target cell antigen interacting domains are ordered according to the following N-terminal to C-terminal arrangements in which (-) is the linker: TVHH-TVHH-EVL-EVH; EVH-EVL-TVHH-TVHH; EVL-EVH-TVHH-TVHH; TVHH-TVHH-EVH-EVL; TVHH-EVL-EVH-TVHH; or TVHH-EVH-EVL-TVHH.

In some embodiments, the effector cell antigen interacting domain is connected to three target cell antigen interacting domains and the variable domains and the three target cell antigen interacting domains are ordered according to the following N-terminal to C-terminal arrangements in which (-) is the linker: TVHH-TVHH-TVHH-EVL-EVH; EVH-EVL-TVHH-TVHH-TVHH; EVL-EVH-TVHH-TVHH-TVHH; TVHH-TVHH-TVHH-EVH-EVL; TVHH-TVHH-EVL-EVH-TVHH; TVHH-EVH-EVL-TVHH-TVHH; TVHH-EVL-EVH-TVHH-TVHH; or TVHH-TVHH-EVH-EVL-TVHH.

In some embodiments, the effector cell antigen interacting domain is connected to four target cell antigen interacting domains and the variable domains and the four target cell antigen interacting domains are ordered according to the following N-terminal to C-terminal arrangements in which (-) is the linker: TVHH-TVHH-TVHH-TVHH-EVL-EVH; EVH-EVL-TVHH-TVHH-TVHH-TVHH; EVL-EVH-TVHH-TVHH-TVHH-TVHH; TVHH-TVHH-TVHH-TVHH-EVH-EVL; TVHH-TVHH-TVHH-EVL-EVH-TVHH; TVHH-EVH-EVL-TVHH-TVHH-TVHH; TVHH-EVL-EVH-TVHH-TVHH-TVHH; TVHH-TVHH-TVHH-EVH-EVL-TVHH; TVHH-TVHH-EVL-EVH-TVHH-TVHH; or TVHH-TVHH-EVH-EVL-TVHH-TVHH.

In some embodiments, the effector cell antigen interacting domain is connected to five target cell antigen interacting domains and the variable domains and the five target cell antigen interacting domains are ordered according to the following N-terminal to C-terminal arrangements in which (-) is the linker: TVHH-TVHH-TVHH-TVHH-TVHH-EVL-EVH; EVH-EVL-TVHH-TVHH-TVHH-TVHH-TVHH; EVL-EVH-TVHH-TVHH-TVHH-TVHH-TVHH; TVHH-TVHH-TVHH-TVHH-TVHH-EVH-EVL; TVHH-TVHH-TVHH-TVHH-EVL-EVH-TVHH; TVHH-EVH-EVL-TVHH-TVHH-TVHH-TVHH; TVHH-EVL-EVH-TVHH-TVHH-TVHH-TVHH; TVHH-TVHH-TVHH-TVHH-EVH-EVL-TVHH; TVHH-TVHH-TVHH-EVL-EVH-TVHH-TVHH; TVHH-TVHH-EVH-EVL-TVHH-TVHH-TVHH; TVHH-TVHH-EVL-EVH-TVHH-TVHH-TVHH; or TVHH-TVHH-TVHH-EVH-EVL-TVHH-TVHH.

In some embodiments, the effector cell antigen interacting domain is connected to six target cell antigen interacting domains and the variable domains and the six target cell antigen interacting domains are ordered according to the following N-terminal to C-terminal arrangements in which (-) is the linker: TVHH-TVHH-TVHH-TVHH-TVHH-TVHH-EVL-EVH; EVH-EVL-TVHH-TVHH-TVHH-TVHH-TVHH-TVHH; EVL-EVH-TVHH-TVHH-TVHH-TVHH-TVHH-TVHH; TVHH-TVHH-TVHH-TVHH-TVHH-TVHH-EVH-EVL; TVHH-TVHH-TVHH-TVHH-TVHH-EVL-EVH-TVHH; TVHH-EVH-EVL-TVHH-TVHH-TVHH-TVHH-TVHH; TVHH-EVL-EVH-TVHH-TVHH-TVHH-TVHH-TVHH; TVHH-TVHH-TVHH-TVHH-TVHH-EVH-EVL-TVHH; TVHH-TVHH-TVHH-TVHH-EVL-EVH-TVHH-TVHH; TVHH-TVHH-EVH-EVL-TVHH-TVHH-TVHH-TVHH; TVHH-TVHH-EVL-EVH-TVHH-TVHH-TVHH-TVHH; TVHH-TVHH-TVHH-TVHH-EVH-EVL-TVHH-TVHH; TVHH-TVHH-TVHH-EVL-EVH-TVHH-TVHH-TVHH; or TVHH-TVHH-TVHH-EVH-EVL-TVHH-TVHH-TVHH.

In some embodiments, the effector cell antigen interacting domain is connected to six target cell antigen interacting domains and the variable domains and the six target cell antigen interacting domains are ordered according to the following N-terminal to C-terminal arrangements in which (-) is the linker: TVHH-TVHH-TVHH-EVH-EVL-TVHH-TVHH-TVHH; or EVH-EVL-TVHH-TVHH-TVHH-TVHH-TVHH-TVHH.

In some embodiments, the effector cell antigen interacting domain is connected to six target cell antigen interacting domains and the variable domains and the six target cell antigen interacting domains are ordered according to the following N-terminal to C-terminal arrangements in which (-) is the linker: TVHH-TVHH-TVHH-EVH-EVL-TVHH-TVHH-TVHH.

In some embodiments, the effector cell antigen interacting domain is connected to six target cell antigen interacting domains and the variable domains and the six target cell antigen interacting domains are ordered according to the following N-terminal to C-terminal arrangements in which (-) is the linker: EVH-EVL-TVHH-TVHH-TVHH-TVHH-TVHH-TVHH.

### Linker Sequences

In certain embodiments, the linkers that connect the two effector cell antigen interacting domains (E) to each other comprise internal linkers.

In certain embodiments, the effector cell antigen interacting domain that is not connected directly to the one or more target cell antigen interacting domains comprises a terminal linker that connects EVL and EVH.

In some embodiments, the effector cell antigen interacting domain comprises a terminal linker that connects EVL and EVH.

In some embodiments, the one or more linkers that connects the two or more target cell antigen interacting domains comprises an internal linker.

In some embodiments, the length of an internal linker is about 4 amino acids to about 30 amino acids. For example, the length of an internal linker is about 5-30, 6-30, 7-30, 8-30, 9-30, 10-30, 5-29, 6-29, 7-29, 8-29, 9-29, 10-29, 5-28, 6-28, 7-28, 8-28, 9-28, 10-28, 5-27, 6-27, 7-27, 8-27, 9-27, 10-27, 5-26, 6-26, 7-26, 8-26, 9-26, 10-26, 5-25, 6-25, 7-25, 8-25, 9-25, 10-25, 5-24, 6-24, 7-24, 8-24, 9-24, 10-24, 5-23, 6-23, 7-23, 8-23, 9-23, 10-23, 5-22, 6-22, 7-22, 8-22, 9-22, 10-22, 5-21, 6-21, 7-21, 8-21, 9-21, 10-21, 5-20, 6-20, 7-20, 8-20, 9-20, 10-20, 5-19, 6-19, 7-19, 8-19, 9-19, 10-19, 5-18, 6-18, 7-18, 8-18, 9-18, 10-18, 5-17, 6-17, 7-17, 8-17, 9-17, 10-17, 5-16, 6-16, 7-16, 8-16, 9-16, 10-16, 5-15, 6-15, 7-15, 8-15, 9-15, or 10-15. In some cases, the internal linker is about 5, 6, 7, 8, 9, 10, 11, 12, 13, 14, or 15 amino acids in length. In certain embodiments, each of the internal linkers is 6-8 amino acids in length.

In some embodiments, the length of terminal linker is about 10 amino acids to about 30 amino acids. For example, the length of a terminal linker is about 10-30, 11-30, 12-30, 13-30, 14-30, 15-30, 10-29, 11-29, 12-29, 13-29, 14-29, 15-29, 10-28, 11-28, 12-28, 13-28, 14-28, 15-28, 10-27, 11-27, 12-27, 13-27, 14-27, 15-27, 10-26, 11-26, 12-26, 13-26, 14-26, 15-26, 10-25, 11-25, 12-25, 13-25, 14-25, 15-25, 10-24, 11-24, 12-24, 13-24, 14-24, 15-24, 10-23, 11-23, 12-23, 13-23, 14-23, 15-23, 10-22, 11-22, 12-22, 13-22, 14-22, 15-22, 10-21, 11-21, 12-21, 13-21, 14-21, 15-21, 10-20, 11-20, 12-20, 13-20, 14-20, 15-20, 10-19, 11-19, 12-19, 13-19, 14-19, 15-19, 10-18, 11-18, 12-18, 13-18, 14-18, 15-18, 10-17, 11-17, 12-17, 13-17, 14-17, 15-17, 10-16, 11-16, 12-16, 13-16, 14-16, or 15-16. In some cases, the terminal linker is about 10, 11, 12, 13, 14, 15, 16, 17, 18, 19, or 20 amino acids in length. In certain embodiments, the terminal linker is 15 or 16 amino acids in length. In some cases, the terminal linker is about 16 amino acids in length.

In some embodiments, the length of the internal linker is about 25 amino acids to about 45 amino acids. For example, the length of the internal linker is about 25-45, 26-44, 27-43, 28-42, 29-41, 30-40, 31-39, 32-38, 33-37, or 34-36 amino acids in length. In some embodiments, the internal linker is about 30, 31, 32, 33, 34, 35, 36, 37, 38, 39, or 40 amino acids in length. In some embodiments, the internal linker is 35 amino acids in length.

In some embodiments, the length of the terminal linker is about 25 amino acids to about 45 amino acids. For example, the length of the terminal linker is about 25-45, 26-44, 27-43, 28-42, 29-41, 30-40, 31-39, 32-38, 33-37, or 34-36 amino acids in length. In some embodiments, the terminal linker is about 30, 31, 32, 33, 34, 35, 36, 37, 38, 39, or 40 amino acids in length. In some embodiments, the terminal linker is 35 amino acids in length.

In some embodiments, the linkers are sized to enforce pairing of the heavy and light chain variable domains to form two effector cell antigen interacting in the single chain polypeptide, eliminate mismatched pairing, prevent aggregation and multiple species during purification, diffusion, and thus facilitate manufacturing.

In some embodiments, the linker comprises a glycine (G) and/or a serine (S). In some embodiments, the linker comprises a sequence as disclosed in Table 1, or a sequence substantially identical thereto (e.g. a sequence with 0-2 amino acid modifications, substitutions or deletions).

TABLE 1
Exemplary linker sequences

| | Amino Acid Sequence | |
|---|---|---|
| Description | (N to C) | SEQ ID NO: |
| Linker-1 (L1) | GGSGGS | 1 |
| Linker-2 (L2) | GGGGSGGGGSGGGGS | 2 |
| Linker-3 (L3) | GGSGGS | 3 |
| Linker-4 (L4) | GGGGSGGGG | 4 |
| Linker-5 (L5) | GGGGSGGGG | 5 |
| Linker-6 (L6) | AAAGS | 6 |

### Effector Cell Antigen Binding Domains

In some embodiments, the effector cell antigen interacting domains bind to an effector cell antigen. In some embodiments, the CD3 is CD3 delta, CD3 gamma, or CD3 epsilon. In some embodiments, the effector cell antigen is on an effector cell. In some embodiments, the effector cell comprises a T cell or a NK cell or a macrophage. In some embodiments, the effector cell antigen comprises CD3, CD16a, or Death Receptor 5 (DR5).

### CD3 Binding Domains

In some embodiments, EVH comprises a variable heavy chain of a single chain variable fragment that binds to CD3, and EVL comprises a variable light chain of a single chain variable fragment that binds to CD3.

In some embodiments, EVH comprises an amino acid sequence according to Table 2, or a sequence substantially identical thereto (e.g., a sequence that has at least 90%, 95%, 96%, 97%, 98%, or 99% sequence identity). In some embodiments, EVH comprises a CDR1 sequence of STYAMN (SEQ ID NO: 13), or a sequence with 0-2 amino acid modifications, substitutions or deletions. In some embodiments, EVH comprises a CDR2 sequence of RIRSKYNNYATYYADSVKD (SEQ ID NO: 14), or a sequence with 0-2 amino acid modifications, substitutions or deletions. In some embodiments, EVH comprises a CDR3 sequence of HGNFGNSYVSWFAY (SEQ ID NO: 15), or a sequence with 0-2 amino acid modifications, substitutions or deletions. In some embodiments, EVH comprises a CDR3 sequence of HGNFGNSYVSYFAY (SEQ ID NO: 16), or a sequence with 0-2 amino acid modifications, substitutions or deletions.

In some embodiments, EVL comprises an amino acid sequence according to Table 3, or a sequence substantially identical thereto (e.g., a sequence that has at least 90%, 95%, 96%, 97%, 98%, or 99% sequence identity). In some embodiments, EVL comprises a CDR1 sequence of RSSTGAVTTSNYAN (SEQ ID NO: 17), or a sequence with 0-2 amino acid modifications, substitutions or deletions. In some embodiments, EVL comprises a CDR2 sequence of GTNKRAP (SEQ ID NO: 18), or a sequence with 0-2 amino acid modifications, substitutions or deletions. In some embodiments, EVL comprises a CDR3 sequence of ALWYSNL (SEQ ID NO: 19), or a sequence with 0-2 amino acid modifications, substitutions or deletions.

TABLE 2
Amino acid sequences of an anti-CD3 variable heavy chain domain (amino acid
sequences of variable heavy CDR1, CDR2 and CDR3 are in bold and underlined)

| | | SEQ ID |
|---|---|---|
| Description | Amino Acid Sequence (N to C) | NO: |
| anti-CD3 VH | EVQLVESGGGLVQPGGSLRLSCAASGFTFSTYAMNWVRQAPGKGL | 7 |
| | EWVGRIRSKYNNYATYYADSVKDRFTISRDDSKNSLYLQMNSLKTE | |
| | DTAVYYCARHGNFGNSYVSYFAYWGQGTLVTVSS | |
| anti-CD3 VH | EVQLVESGGGLVQPGGSLRLSCAASGFTFSTYAMNWVRQAPGKGL | 8 |
| | EWVGRIRSKYNNYATYYADSVKDRFTISRDDSKNSLYLQMNSLKTE | |
| | DTAVYYCARHGNFGNSYVSWFAYWGQGTLVTVSS | |
| anti-CD3 VH | EVQLVESGGGLVQPGGSLKLSCAASGFTFNKYAINWVRQAPGKGLE | 75 |
| | WVARIRSKYNNYATYYADQVKDRFTISRDDSKNTAYLQMNNLKTE | |
| | DTAVYYCVRHANFGNSYISYWAYWGQGTLVTVSS | |
| anti-CD3 VH | EVQLVESGGGLVQPGRSLRLSCAASGFTFDDYAMHWVRQAPGKGL | 77 |
| | EWVSGISWNSGSIGYADSVKGRFTISRDNAKNSLYLQMNSLRAEDT | |
| | ALYYCAKDSRGYGDYRLGGAYWGQGTLVTVSS | |
| anti-CD3 VH | EVQLVESGGGLVQPGGSLKLSCAASGFTFNKYAMNWVRQAPGKGL | 86 |
| | EWVARIRSKYNNYATYYADSVKDRFTISRDDSKNTAYLQMNNLKTE | |
| | DTAVYYCVRHGNFGNSYISYWAYWGQGTLVTVSS | |

TABLE 3
Amino acid sequences of an anti-CD3 variable
light chain domain (amino acid sequences of
variable light chain CDR1, CDR2 and CDR3
are in bold and underlined)

| | | SEQ ID |
|---|---|---|
| Description | Amino Acid Sequence (N to C) | NO: |
| anti-CD3 VL | DIQMTQSPSSLSASVGDRVTITCRSSTGAVTTSNYANWVQQKPGKA | 9 |
| | PKALIGGTNKRAPGVPSRFSGSLIGDKATLTISSLQPEDFATYYCAL | |
| | WYSNLWVFGQGTKVEIK | |
| anti-CD3 VL | DIQMTQSPSSLSASVGDRVTITCRSSTGAVTTSNYANWVQQKPGKA | 10 |
| | PKGLIGGTNKRAPGVPARFSGSGSGTDFTLTISSLQPEDFATYYCAL | |
| | WYSNLWVFGQGTKVEIK | |
| anti-CD3 VL | DIQMTQSPSSLSASVGDRVTITCRSSTGAVTTSNYANWVQQKPGKA | 11 |
| | PKGLIGGTNKRAPGVPSRFSGSLIGDKATLTISSLQPEDFATYYCAL | |
| | WYSNLWVFGQGTKVEIK | |
| anti-CD3 VL | DIQMTQSPSSLSASVGDRVTITCRSSTGAVTTSNYANWVQQKPGKA | 12 |
| | PKGLIGGTNKRAPGVPSRFSGSLIGTDFTLTISSLQPEDFATYYCAL | |
| | WYSNLWVFGQGTKVEIK | |
| anti-CD3 VL | QTVVTQEPSLTVSPGGTVTLTCASSTGAVTSGNYPNWVQQKPGQAPR | 76 |
| | GLIGGTKFLVPGTPARFSGSLLGGKAALTLSGVQPEDEAEYYCTLWY | |
| | SNRWVFGGGTKLTVL | |
| anti-CD3 VL | EIVMTQSPATLSVSPGERATLSCRASQSVSSNLAWYQQKPGQAPRLL | 78 |
| | IYGASTRATGIPARFSGSGSGTEFTLTISSLQSEDFAVYYCQQYNNW | |
| | PWTFGQGTKVEIK | |
| anti-CD3 VL | QTVVTQEPSLTVSPGGTVTLTCGSSTGAVTSGNYPNWVQQKPGQAP | 87 |
| | RGLIGGTKFLAPGTPARFSGSLLGGKAALTLSGVQPEDEAEYYCVLW | |
| | YSNRWVFGGGTKLTVL | |

In some instances, the CDR1, CDR2, CDR3 from EVH or EVL is derived from other CD3 antibodies, such as, for example UCHT1, muromonab-CD3 (OKT3), otelixizumab (TRX4), teplizumab (MGA031), visilizumab (Nuvion), or the like.

In some aspects, described herein are anti-CD3 binding molecules comprising an anti-CD3 binding domain that is humanized or fully human, i.e. of human origin. In some embodiments, described herein are anti-CD3 binding molecules comprising an anti-CD3 binding domain that is camelid or llama.

In certain instances, a CD3 binding site has intermediate or low affinity as a monomer and higher affinity and/or specificity as a bivalent molecule due to avidity.

### CD16a Binding Domains

In some embodiments, EVH comprises a variable heavy chain of a single chain variable fragment that binds to CD16a, and EVL comprises a variable light chain of a single chain variable fragment that binds to CD16a.

In some embodiments, EVH comprises an amino acid sequence according to SEQ ID NO: 20 or a sequence substantially identical thereto (e.g., a sequence that has at least 90%, 95%, 96%, 97%, 98%, or 99% sequence identity).

In some embodiments, EVL comprises an amino acid sequence according to SEQ ID NO: 21 or a sequence substantially identical thereto (e.g., a sequence that has at least 90%, 95%, 96%, 97%, 98%, or 99% sequence identity).

In some aspects, described herein are anti-CD16a binding molecules comprising an anti-CD16a binding domain that is humanized or fully human, i.e. of human origin.

TABLE 4
Amino acid sequences of an anti-CD16a
VH or VL domain

| | Amino Acid Sequence | SEQ ID |
|---|---|---|
| Description | (N to C) | NO: |
| Anti-CD16a | QVQLVQSGAEVKKPGESLKVSCK | 20 |
| VH | ASGYTFTSYYMHWVRQAPGQGLE | |
| | WMGIINPSGGSTSYAQKFQGRVT | |
| | MTRDTSTSTVYMELSSLRSEDTA | |
| | VYYCARGSAYYYDFADYWGQGTL | |
| | VTVSS | |
| Anti-CD16a | SYVLTQPSSVSVAPGQTATISCG | 21 |
| VL | GHNIGSKNVHWYQQRPGQSPVLV | |
| | IYQDNKRPSGIPERFSGSNSGNT | |
| | ATLTISGTQAMDEADYYCQVWDN | |
| | YSVLFGGGTKLTVL | |

### Death Receptor 5 Binding Domains

In some embodiments, EVH comprises a van able heavy chain of a single chain variable fragment that binds to DR5, and EVL comprises a variable light chain of a single chain variable fragment that binds to DR5.

In some embodiments, EVH comprises an amino acid sequence according to SEQ ID NO: 22-26 or a sequence substantially identical thereto (e.g., a sequence that has at least 90%, 95%, 96%, 97%, 98%, or 99% sequence identity).

In some embodiments, EVL comprises an amino acid sequence according to SEQ ID NO: 27-31 or a sequence substantially identical thereto (e.g., a sequence that has at least 90%, 95%, 96%, 97%, 98%, or 99% sequence identity).

In some aspects, described herein are anti-DR5 binding molecules comprising an anti-DR5 binding domain that is humanized or fully human, i.e. of human origin.

TABLE 5
Amino acid sequences of an anti-DR5 VH or VL domain

| | | SEQ ID |
|---|---|---|
| Description | Amino Acid Sequence (N to C) | NO: |
| Anti-DR5 | EVQLLESGGGLVQPGGSLRLSCAASGFTFSSYAMSWVRQAPGKGLE | 22 |
| VH | WVSAISGSGGSTYYADSVKGRFTISRDNSKNTLYLQMNSLRAEDTA | |
| | VYYCAKGVRISFDYWGQGTLVTVSS | |
| Anti-DR5 | EVQLLESGGGLVQPGGSLRLSCAASGFTFSSYAMSWVRQAPGKGLE | 23 |
| VH | WVSAISGSGGSTYYADSVKGRFTISRDNSKNTLYLQMNSLRAEDMA | |
| | VYYCAKGARVSFDYWGQG TLVTVSS | |
| Anti-DR5 | EVQLLESGGGLVQPGGSLRLSCAASGFTFSSYAMSWVRQAPGKGLE | 24 |
| VH | WVSAISGSGGSTYYADSVKGRFTISRDNSKNTLYLQMNSLRAEDTA | |
| | VYYCAKGVRKGFDYWGQGTLVTVSS | |
| Anti-DR5 | EVQLLESGGGLVQPGGSLRLSCAASGFTFSSYAMSWVRQAPGKGLE | 25 |
| VH | WVSAISGSGGSTYYADSVKGRFTISRDNSKNTLYLQMNSLRAEDTA | |
| | VYYCAKGVRVSFDYWGQG TLVTVSS | |
| Anti-DR5 | EVQLLESGGGLVQPGGSLRLSCAASGFTFSSYAMSWVRQAPGKGLE | 26 |
| VH | WVSAISGSGGSTYYADSVKGRFTISRDNSKNTLYLQMNSLRAEDAA | |
| | VYYCAKGVRKKFDYWGQGTLVTVSS | |
| Anti-DR5 | EIVLTQSPGTLSLSPGERATLSCRASQSVSSSYLAWYQQKPGQAPR | 27 |
| VL | LLIYGASSRATGIPDRFSGSGSGTDFTLTISRLEPEDFAVYYCQQG | |
| | SNQPVTFGQGTKVEIK | |
| Anti-DR5 | EIVLTQSPGT LSLSPGERAT LSCRASQSVS SSYLAWYQQK | 28 |
| VL | PGQAPRLLIYGASSRATGIP DRFSGSGSGT DFTLTISRLE | |
| | PEDFAVYYCQ QGSQPPITFGQGTKVEIK | |
| Anti-DR5 | EIVLTQSPGT LSLSPGERAT LSCRASQSVS SSYLAWYQQK | 29 |
| VL | PGQAPRLLIYGASSRATGIP DRFSGSGSGT DFTLTISRLE | |
| | PEDFAVYYCQ QGESPPPTFGQGTKVEIK | |
| Anti-DR5 | EIVLTQSPGT LSLSPGERAT LSCRASQSVS SSYLAWYQQK | 30 |
| VL | PGQAPRLLIYGASSRATGIP DRESGSGSGT DFTLTISRLE | |
| | PEDFAVYYCQ QGTTHPITFGQGTKVEIK | |
| Anti-DR5 | EIVLTQSPGT LSLSPGERAT LSCRASQSVS SSYLAWYQQK | 31 |
| VL | PGQAPRLLIYGASSRATGIP DRESGSGSGT DFTLTISRLE | |
| | PEDFAVYYCQ QGQLPPITFGQGTKVEIK | |

### Target Cell Antigen Interacting Domains

In some embodiments, the target cell antigen interacting domains that interact with a target cell (T) bind to a target cell antigen. In some embodiments, the target cell antigen is clustered in a lipid raft when on a cell associated with a disease state and not clustered in a lipid raft when on a cell that is not associated with a disease state. In some embodiments, the target cell antigen is clustered when on a cell associated with a disease state and not clustered when on a cell that is not associated with a disease state. In some embodiments, the target cell antigens are 100 nm or less when on a cell associated with a disease state and not within 100 nm when on a cell that is not associated with a disease state. In some embodiments, the target cell antigens are 75 nm or less when on a cell associated with a disease state and not within 75 nm when on a cell that is not associated with a disease state. In some embodiments, the target cell antigens are 50 nm or less when on a cell associated with a disease state and not within 50 nm when on a cell that is not associated with a disease state. In some embodiments, the target cell antigens are 25 nm or less when on a cell associated with a disease state and not within 25 nm when on a cell that is not associated with a disease state. In some embodiments, the target cell antigens are 10 nm or less when on a cell associated with a disease state and not within 10 nm when on a cell that is not associated with a disease state. In some embodiments, the target cell antigens are 5 nm or less when on a cell associated with a disease state and not within 5 nm when on a cell that is not associated with a disease state. In some embodiments, the target cell antigens are 2 nm or less when on a cell associated with a disease state and not within 2 nm when on a cell that is not associated with a disease state.

In some embodiments, the target cell antigens are in close proximity when on a cell associated with a disease state and not within close proximity when on a cell that is not associated with a disease state. In some embodiments, the target cell antigen is a dimer, trimer, tetramer, or oligomer when on a cell associated with a disease state and a monomer or dimer when on a cell that is not associated with a disease state. In some embodiments, the target cell antigen is part of a cell signaling complex when on a cell associated with a disease state and not part of the same cell signaling complex when on a cell that is not associated with a disease state. In some embodiments, the target cell antigen is part of a cell signaling complex when on a cell associated with a disease state and not part of a cell signaling complex when on a cell that is not associated with a disease state. In some embodiments, the cell associated with a disease state is a myeloid-cell, fibroblast, or cancer cell. In some embodiments, the target cell antigen comprises CD33, FAP, EGFR, HER2, or EpCAM.

### CD33 Binding Domains

In some embodiments, TVHH comprises a VHH single domain antibody that binds to CD33. In some embodiments, TVHH comprises an amino acid sequence according to SEQ ID NO: 32-43, 72-74 or a sequence substantially identical thereto (e.g., a sequence that has at least 90%, 95%, 96%, 97%, 98%, or 99% sequence identity).

TABLE 6
Amino acid sequences of an anti-CD33 VHH single domain antibody

| | | SEQ ID |
|---|---|---|
| Description | Amino Acid Sequence (N to C) | NO: |
| Anti-CD33 | QVQLQESGGGLVQPGGSLRLSCAASGFTFGSYDMAWVRQAPGKGP | 32 |
| VHH | EWVSSINSSGGSTEYASSVKGRFTVSRDNAKNMLYLQMDRLKLEDT | |
| | AVYYCADEGNPSTTWYYEDQGTQVTVSS | |
| Anti-CD33 | QVQLQESGGGLVQAGGSLRLSCAASGSIGSINFIFWYRQAPGKQREF | 33 |
| VHH | VARISRSGRSDYVESVKGRFTISRDNAKNTVYLQMNNLKSEDTAVY | |
| | YCALGNNWGQGTQVTVSS | |
| Anti-CD33 | QVQLQESGGGLVQPGGSLRLSCVASRSIFSNSIITSADHTNYRDAVKG | 34 |
| VHH | RFTISRDSAKNTVYLQMNSLKPEDTAVYFCADGRYWGQGTQVTVSS | |
| Anti-CD33 | QVQLQESGGGLVQAGGSLRLSCAASESAFNFYTMGWHRQAPGKQR | 35 |
| VHH | ELVASITSGGDTNYANFVEGRFTISRDNAKNTVSLQMNSLNPEDTAI | |
| | YYCDGKNPRSSSSTWYWGQGTQVTVSS | |
| Anti-CD33 | QVQLQESGGGLVQAGGSLRLSCAASTGIFRATVMGWYRQPPGKQRE | 36 |
| VHH | LVARLVSGGSTRYADSVKGRVTISRDNAKNTVYLQINSLKPEDTAV | |
| | YYCDGIIMNRTYWGQGTQVTVSS | |
| Anti-CD33 | QVQLQESGGGLVQAGGSLRLSCASSGSILSMNVMGWYRQAPGKQR | 37 |
| VHH | EMVAQITRIGDTNYSSSMKGRFTISRDNADNTLYLQMNRLEPEDTAV | |
| | YFCANNNRSTYYYYWGQGTQVTVSS | |
| Anti-CD33 | QVQLQESGGGLVQAGGSLRLSCTALRSVGSFNGMGWYRQSPGNQR | 38 |
| VHH | ELVATVTSDTSTTYRDSVKGRFAISRDSAKTVSLQMNNLKPEDTAVY | |
| | YCADEEGHHLLNRRSTVWYWGQGTQVTVSS | |
| Anti-CD33 | QVQLQESGGGLVQAGGSLRLSCTAFRSVGVIDVMGWYRQAPGTQR | 39 |
| VHH | ELVATVTSSSSTTYADSVKGRFAISRDDAKTVSLQMNSLKPEDTAVY | |
| | YCADDEEGLLNNRRSSVWYWGQGTQVTVSS | |
| Anti-CD33 | QVQLQESGGGLVQAGGSLRLSCTAFRSVGVIDVMGWYRQAPGKQR | 40 |
| VHH | ELVATVTSGSSTTYADSVKGRFAISRDNAKTVSLQMNSLKPEDTAVY | |
| | YCADDDEGKLLNRRSVWYYWSQGTQVTVSS | |
| Anti-CD33 | QVQLQESGGGLVQAGESLRLSCAASQKIFNNMGWYRQAPGKEREL | 41 |
| VHH | VAEISRGGDTNYAAFTKDRFTISRDNAKNTVYLQMNSLKPEDTAVY | |
| | YCADGHILPSTVWYWGQGTQVTVSS | |
| Anti-CD33 | QVQLQESGGGVVQAGGSLRLSCAPNGGMFVFDAMGWYRQAPGKQ | 42 |
| VHH | RELVATITRSSRRDYADSVKGRFTISKDAAKNTVYLQMNSLKPEDTA | |
| | VYYCAADGGHNNRSVYYYWGQGTQVTVSS | |
| Anti-CD33 | QVQLQESGGGLVQAGGSLRLSCAASGILFSSYLMGWYRQAPGKQRE | 43 |
| VHH | LVAQISADMDTSYVNSVKGRFTISRDNAENTVYLQMNSLEPEDTAV | |
| | YYCAAFGGKNPRSSTTWYWGQGTQVTVSS | |
| Anti-CD33 | EVQLVESGGGEVQPGGSLRLSCAASGSGFSASLMSWHRQAPGKQRD | 72 |
| VHH | LVASITRDGRANYVESVKGRFTISRDNAKNTLYLQMSSLRAEDTAV | |
| | YYCHAYSFDYPIRSYWGQGTLVTVSS | |
| Anti-CD33 | EVQLVESGGGEVQPGGSLRLSCAASGSIFSISIMGWYRQAPGKEREL | 73 |
| VHH | VASTTSSGTTNYVESVKGRFTISRDNAKNTLYLQMSSLRAEDTAVYY | |
| | CHAYIATTTDRGYRGYWGQGTLVTVSS | |
| Anti-CD33 | EVQLVESGGGEVQPGGSLRLSCAASGFTFSSYAMGWFRQAPGKGRE | 74 |
| VHH | WVAAITTSGDTTYYAESVKGRFTISRDNAKNTVYLQMSSLRAEDTA | |
| | VYYCAAHRGGGVIDYWGQGTLVTVSS | |

### HER2 Binding Domains

In some embodiments, TVHH comprises a VHH single domain antibody that binds to HER2. In some embodiments, TVHH comprises an amino acid sequence according to SEQ ID NO: 44-46 or a sequence substantially identical thereto (e.g., a sequence that has at least 90%, 95%, 96%, 97%, 98%, or 99% sequence identity).

TABLE 7
Amino acid sequences of an anti-HER2
VHH single domain antibody

| | Amino Acid Sequence | SEQ ID |
|---|---|---|
| Description | (N to C) | NO: |
| Anti-HER2 | EVQLVESGGSLVQPGGSLRL | 44 |
| VHH | SCAASGFTFDDYAMSWVRQV | |
| | PGKGLEWVSSINWSGTHTDY | |
| | ADSVKGRFTISRNNANNTLY | |
| | LQMNSLKSEDTAVYYCAKNW | |
| | RDAGTTWFEKSGSAGQGTQV | |
| | TVSS | |
| Anti-HER2 | EVQLVESGGGLVQAGGSLRL | 45 |
| VHH | SCAASGITFSINTMGWYRQA | |
| | PGKQRELVALISSIGDTYYA | |
| | DSVKGRFTISRDNAKNTVYL | |
| | QMNSLKPEDTAVYYCKRFRT | |
| | MQGTDYWGQGTQVTVSS | |
| Anti-HER2 | KVQLVESGGGLVQPGGSLRL | 46 |
| VHH | SCAASGSIFGFNDMAWYRQA | |
| | PGKQRELVALISRVGVTSSA | |
| | DSVKGRFTISRVNAKDTVYL | |
| | QMNSLKPEDTAVYYCYMDQR | |
| | LDGSTLAYWGQGTQVTVSS | |

### EGFR Binding Domains

In some embodiments, TVHH comprises a VHH single domain antibody that binds to EGFR. In some embodiments, TVHH comprises an amino acid sequence according to SEQ ID NO: 47-49 or a sequence substantially identical thereto (e.g., a sequence that has at least 90%, 95%, 96%, 97%, 98%, or 99% sequence identity).

TABLE 8
Amino acid sequences of an anti-EGFR
VHH single domain antibody

| | Amino Acid Sequence | SEQ ID |
|---|---|---|
| Description | (N to C) | NO: |
| Anti-EGFR | QVKLEESGGGSVQTGGSLRLTCAAS | 47 |
| VHH | GRTSRSYGMGWFRQAPGKEREFVSG | |
| | ISWRGDSTGYADSVKGRFTISRDNA | |
| | KNTVDLQMNSLKPEDTAIYYCAAAA | |
| | GSAWYGTLYEYDYWGQGTQVTVSS | |
| Anti-EGFR | AVQLVESGGGSVQAGGSLRLTCAAS | 48 |
| VHH | GRTSRSYGMGWFRQAPGKEREFVSG | |
| | ISWRGDSTGYADSVKGRFTISRDNA | |
| | KNTVDLQMNSLKPEDTAIYYCAAAA | |
| | GSTWYGTLYEYDYWGQGTQVTVSS | |
| Anti-EGFR | EVQLVESGGGLVQAGGSLRLSCAAS | 49 |
| VHH | GRTFSSYAMGWFRQAPGKEREFVVA | |
| | INWSSGSTYYADSVKGRFTISRDNA | |
| | KNTMYLQMNSLKPEDTAVYYCAAGY | |
| | QINSGNYNFKDYEYDYWGQGTQVTV | |
| | SS | |
| Anti-EGFR | EVQLVESGGGLVQAGGSLRLSCAAS | 50 |
| VHH | GRTFSSYVMGWFRQATGKEREFVAT | |
| | IAWDSGSTYYADSVKGRFTISRDNA | |
| | KNTVHLQMNSLKPEDTAVYYCAASY | |
| | NVYYNNYYYPISRDEYDYWGQGTQV | |
| | TVSS | |

### FAP Binding Domains

In some embodiments, TVHH comprises a VHH single domain antibody that binds to FAP. In some embodiments, TVHH comprises an amino acid sequence according to SEQ ID NO: 51, 65-67 or a sequence substantially identical thereto (e.g., a sequence that has at least 90%, 95%, 96%, 97%, 98%, or 99% sequence identity).

TABLE 9
Amino acid sequences of an anti-FAP
VHH single domain antibody

| | Amino Acid Sequence | SEQ ID |
|---|---|---|
| Description | (N to C) | NO: |
| Anti-FAP | QVQLQESGGGSVQLRLSCAASGYTV | 51 |
| VHH | RSSYMGWFRQVPGKQREAVAIITSG | |
| | GTTYYADSVKGRFTISRDNAKNTLY | |
| | LSLKPEDTAMYYCAGRTGFIGGIWF | |
| | RDRDYDYWGQGTQVTVSS | |
| Anti-FAP | QVQLQESGGGLVQTGGSLRLSCAAS | 65 |
| VHH | GSIFVGNAMGWYRQALGNQRELVAG | |
| | ITSDGTTYYPDSVKGRFTISRDNDK | |
| | NTIYLQMNSLKPEDTAVYYCNLWPP | |
| | RIGFASWGQGTQVTVSS | |
| Anti-FAP | QVQLQESGGGLVQAGGSLRLSCWSG | 66 |
| VHH | TILSSNSMGWYRQAPGKRRELVASI | |
| | STDGSTNYADSVKGRFTISRDNAKS | |
| | TVFLQMNSLKPEDTAVYYCNFHPPW | |
| | RDWGDTYWGTQVTVSS | |
| Anti-FAP | QVQLQESGGGLVQAGGSLRLSCVAS | 67 |
| VHH | GTIFSSGAMAMGWYRQAPGKQREWV | |
| | AGITGSRTTTYADSVKGRFTISRDN | |
| | AENTVFLQMNNLKSEDTAVYYCNLW | |
| | PPSRPDHWGQGTQVTVSS | |

### EpCAM Binding Domains

In some embodiments, TVHH comprises a VHH single domain antibody that binds to EpCAM. In some embodiments, TVHH comprises an amino acid sequence according to SEQ ID NO: 68-71 or a sequence substantially identical thereto (e.g., a sequence that has at least 90%, 95%, 96%, 97%, 98%, or 99% sequence identity).

TABLE 10
Amino acid sequences of an anti-EpCAM
VHH single domain antibody

| | Amino Acid Sequence | SEQ ID |
|---|---|---|
| Description | (N to C) | NO: |
| Anti-EpCAM | QVQLVESGGGLVQPGGSLTLSCAAS | 68 |
| VHH | GTGSIFSINLMGWYRQAPGKQRELV | |
| | ARITSGDSTVYADSVKGRFTISRDN | |
| | SKNTLYLQMNSLRPEDTAVYYCNLL | |
| | LRSSPGATTPYWGQGTLVTVSS | |
| Anti-EpCAM | QVQLVESGGGLVQPGGSLTLSCVIS | 69 |
| VHH | GSFSALWAMRWYRQAPGQQRELVAS | |
| | SRGGTTSYADSVKGRFTISRDNSKN | |
| | TLYLQMNSLRPEDTAVYYCNAIDGH | |
| | LAYWGQGTLVTVSS | |
| Anti-EpCAM | QVQLLESGGGLVQPGGSLRLSCAAS | 70 |
| VHH | GRTFSDYDMGWFRQGPGKEREFVAA | |
| | ISWSGGHTNYADSVKGRFTISRDNS | |
| | KNTLYLQMNSLRAEDTAVYYCAADL | |
| | RFTGGDTTTPETYDYWGQGTLVTVS | |
| | S | |
| Anti-EpCAM | QVQLVESGGGLVQPGRSLRLSCAAS | 71 |
| VHH | GRTLDNYDMGWFRQGPGKEREFVAA | |
| | ISWSGGSTDYAYSVTGRFTISRDNA | |
| | KNSLYLQMNSLRAEDTALYYCAADL | |
| | RFTGGDTMTPETYDYWGQGTLVTVS | |
| | S | |

### Exemplary Single Chain Polypeptide (SCP) Compositions

### SCP 1

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: EVH-EVL-EVH-EVL-TVHH-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 52. A summary of the amino acid sequences of the domains of SCP 1 are provided in Table 11.

TABLE 11
Summary of Sequences for SCP 1

| | Amino Acid Sequence | SEQ ID |
|---|---|---|
| Description | (N to C) | NO: |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L1 | GGSGGS | 1 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L2 | GGGGSGGGGSGGGGS | 2 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L3 | GGSGGS | 3 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L4 | GGGGSGGGG | 4 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L5 | GGGGSGGGG | 5 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L6 | AAAGS | 6 |
| Purification | HHHHHH | 64 |
| Tag | | |
| Full length | EVQLVESGGGLVQPGGSLRLSCAAS | 52 |
| sequence of | GFTFSTYAMNWVRQAPGKGLEWVGR | |
| SCP 1 | IRSKYNNYATYYADSVKDRFTISRD | |
| | DSKNSLYLQMNSLKTEDTAVYYCAR | |
| | HGNFGNSYVSYFAYWGQGTLVTVSS | |
| | GGSGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGGGSGGGG | |
| | SGGGGSEVQLVESGGGLVQPGGSLR | |
| | LSCAASGFTFSTYAMNWVRQAPGKG | |
| | LEWVGRIRSKYNNYATYYADSVKDR | |
| | FTISRDDSKNSLYLQMNSLKTEDTA | |
| | VYYCARHGNFGNSYVSYFAYWGQGT | |
| | LVTVSSGGSGGSDIQMTQSPSSLSA | |
| | SVGDRVTITCRSSTGAVTTSNYANW | |
| | VQQKPGKAPKALIGGTNKRAPGVPS | |
| | RFSGSLIGDKATLTISSLQPEDFAT | |
| | YYCALWYSNLWVFGQGTKVEIKGGG | |
| | GSGGGGQVQLQESGGGLVQPGGSLR | |
| | LSCAASGFTFGSYDMAWVRQAPGKG | |
| | PEWVSSINSSGGSTEYASSVKGRFT | |
| | VSRDNAKNMLYLQMDRLKLEDTAVY | |
| | YCADEGNPSTTWYYEDQGTQVTVSS | |
| | GGGGSGGGGQVQLQESGGGLVQPGG | |
| | SLRLSCAASGFTFGSYDMAWVRQAP | |
| | GKGPEWVSSINSSGGSTEYASSVKG | |
| | RFTVSRDNAKNMLYLQMDRLKLEDT | |
| | AVYYCADEGNPSTTWYYEDQGTQVT | |
| | VSSAAAGSHHHHHH | |

### SCP 2

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: EVL-EVH-EVL-EVH-TVHH-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 53. A summary of the amino acid sequences of the domains of SCP 2 are provided in Table 12.

TABLE 12
Summary of Sequences for SCP 2

| | Amino Acid Sequence | SEQ ID |
|---|---|---|
| Description | (N to C) | NO: |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L1 | GGSGGS | 1 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L2 | GGGGSGGGGSGGGGS | 2 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L3 | GGSGGS | 3 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L4 | GGGGSGGGG | 4 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L5 | GGGGSGGGG | 5 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L6 | AAAGS | 6 |
| Purification | HHHHHH | 64 |
| Tag | | |
| Full length | DIQMTQSPSSLSASVGDRVTITCRS | 53 |
| sequence of | STGAVTTSNYANWVQQKPGKAPKAL | |
| SCP 2 | IGGTNKRAPGVPSRFSGSLIGDKAT | |
| | LTISSLQPEDFATYYCALWYSNLWV | |
| | FGQGTKVEIKGGSGGSEVQLVESGG | |
| | GLVQPGGSLRLSCAASGFTFSTYAM | |
| | NWVRQAPGKGLEWVGRIRSKYNNYA | |
| | TYYADSVKDRFTISRDDSKNSLYLQ | |
| | MNSLKTEDTAVYYCARHGNFGNSYV | |
| | SYFAYWGQGTLVTVSSGGGGSGGGG | |
| | SGGGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGSGGSEVQ | |
| | LVESGGGLVQPGGSLRLSCAASGFT | |
| | FSTYAMNWVRQAPGKGLEWVGRIRS | |
| | KYNNYATYYADSVKDRFTISRDDSK | |
| | NSLYLQMNSLKTEDTAVYYCARHGN | |
| | FGNSYVSYFAYWGQGTLVTVSSGGG | |
| | GSGGGGQVQLQESGGGLVQPGGSLR | |
| | LSCAASGFTFGSYDMAWVRQAPGKG | |
| | PEWVSSINSSGGSTEYASSVKGRFT | |
| | VSRDNAKNMLYLQMDRLKLEDTAVY | |
| | YCADEGNPSTTWYYEDQGTQVTVSS | |
| | GGGGSGGGGQVQLQESGGGLVQPGG | |
| | SLRLSCAASGFTFGSYDMAWVRQAP | |
| | GKGPEWVSSINSSGGSTEYASSVKG | |
| | RFTVSRDNAKNMLYLQMDRLKLEDT | |
| | AVYYCADEGNPSTTWYYEDQGTQVT | |
| | VSSAAAGSHHHHHH | |

### SCP 3

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: EVH-EVL-EVH-EVL-TVHH-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 54. A summary of the amino acid sequences of the domains of SCP 3 are provided in Table 13.

TABLE 13
Summary of Sequences for SCP 3

| | Amino Acid Sequence | SEQ ID |
|---|---|---|
| Description | (N to C) | NO: |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L1 | GGSGGS | 1 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L2 | GGGGSGGGGSGGGGS | 2 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L3 | GGSGGS | 3 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L4 | GGGGSGGGG | 4 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L5 | GGGGSGGGG | 5 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L6 | AAAGS | 6 |
| Purification | HHHHHH | 64 |
| Tag | | |
| Full length | EVQLVESGGGLVQPGGSLRLSCAAS | 54 |
| sequence of | GFTFSTYAMNWVRQAPGKGLEWVGR | |
| SCP 3 | IRSKYNNYATYYADSVKDRFTISRD | |
| | DSKNSLYLQMNSLKTEDTAVYYCAR | |
| | HGNFGNSYVSYFAYWGQGTLVTVSS | |
| | GGSGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGGGSGGGG | |
| | SGGGGSEVQLVESGGGLVQPGGSLR | |
| | LSCAASGFTFSTYAMNWVRQAPGKG | |
| | LEWVGRIRSKYNNYATYYADSVKDR | |
| | FTISRDDSKNSLYLQMNSLKTEDTA | |
| | VYYCARHGNFGNSYVSYFAYWGQGT | |
| | LVTVSSGGSGGSDIQMTQSPSSLSA | |
| | SVGDRVTITCRSSTGAVTTSNYANW | |
| | VQQKPGKAPKALIGGTNKRAPGVPS | |
| | RFSGSLIGDKATLTISSLQPEDFAT | |
| | YYCALWYSNLWVFGQGTKVEIKGGG | |
| | GSGGGGQVQLQESGGGLVQAGGSLR | |
| | LSCAASGSIGSINFIFWYRQAPGKQ | |
| | REFVARISRSGRSDYVESVKGRFTI | |
| | SRDNAKNTVYLQMNNLKSEDTAVYY | |
| | CALGNNWGQGTQVTVSSGGGGSGGG | |
| | GQVQLQESGGGLVQAGGSLRLSCAA | |
| | SGSIGSINFIFWYRQAPGKQREFVA | |
| | RISRSGRSDYVESVKGRFTISRDNA | |
| | KNTVYLQMNNLKSEDTAVYYCALGN | |
| | NWGQGTQVTVSSAAAGSHHHHHH | |

### SCP 4

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: EVL-EVH-EVL-EVH-TVHH-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 55. A summary of the amino acid sequences of the domains of SCP 4 are provided in Table 14.

TABLE 14
Summary of Sequences for SCP 4

| | Amino Acid Sequence | SEQ ID |
|---|---|---|
| Description | (N to C) | NO: |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L1 | GGSGGS | 1 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L2 | GGGGSGGGGSGGGGS | 2 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L3 | GGSGGS | 3 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L4 | GGGGSGGGG | 4 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L5 | GGGGSGGGG | 5 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L6 | AAAGS | 6 |
| Purification | HHHHHH | 64 |
| Tag | | |
| Full length | DIQMTQSPSSLSASVGDRVTITCRS | 55 |
| sequence of | STGAVTTSNYANWVQQKPGKAPKAL | |
| SCP 4 | IGGTNKRAPGVPSRFSGSLIGDKAT | |
| | LTISSLQPEDFATYYCALWYSNLWV | |
| | FGQGTKVEIKGGSGGSEVQLVESGG | |
| | GLVQPGGSLRLSCAASGFTFSTYAM | |
| | NWVRQAPGKGLEWVGRIRSKYNNYA | |
| | TYYADSVKDRFTISRDDSKNSLYLQ | |
| | MNSLKTEDTAVYYCARHGNFGNSYV | |
| | SYFAYWGQGTLVTVSSGGGGSGGGG | |
| | SGGGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGSGGSEVQ | |
| | LVESGGGLVQPGGSLRLSCAASGFT | |
| | FSTYAMNWVRQAPGKGLEWVGRIRS | |
| | KYNNYATYYADSVKDRFTISRDDSK | |
| | NSLYLQMNSLKTEDTAVYYCARHGN | |
| | FGNSYVSYFAYWGQGTLVTVSSGGG | |
| | GSGGGGQVQLQESGGGLVQAGGSLR | |
| | LSCAASGSIGSINFIFWYRQAPGKQ | |
| | REFVARISRSGRSDYVESVKGRFTI | |
| | SRDNAKNTVYLQMNNLKSEDTAVYY | |
| | CALGNNWGQGTQVTVSSGGGGSGGG | |
| | GQVQLQESGGGLVQAGGSLRLSCAA | |
| | SGSIGSINFIFWYRQAPGKQREFVA | |
| | RISRSGRSDYVESVKGRFTISRDNA | |
| | KNTVYLQMNNLKSEDTAVYYCALGN | |
| | NWGQGTQVTVSSAAAGSHHHHHH | |

### SCP 5

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: TVHH-TVHH-EVH-EVL-EVH-EVL. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 56. A summary of the amino acid sequences of the domains of SCP 5 are provided in Table 15.

TABLE 15
Summary of Sequences for SCP 5

| | Amino Acid Sequence | SEQ ID |
|---|---|---|
| Description | (N to C) | NO: |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L5 | GGGGSGGGG | 5 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L4 | GGGGSGGGG | 4 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L3 | GGSGGS | 3 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L2 | GGGGSGGGGSGGGGS | 2 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L1 | GGSGGS | 1 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L6 | AAAGS | 6 |
| Purification | HHHHHH | 64 |
| Tag | | |
| Full length | QVQLQESGGGLVQPGGSLRLSCAAS | 56 |
| sequence of | GFTFGSYDMAWVRQAPGKGPEWVSS | |
| SCP 5 | INSSGGSTEYASSVKGRFTVSRDNA | |
| | KNMLYLQMDRLKLEDTAVYYCADEG | |
| | NPSTTWYYEDQGTQVTVSSGGGGSG | |
| | GGGQVQLQESGGGLVQPGGSLRLSC | |
| | AASGFTFGSYDMAWVRQAPGKGPEW | |
| | VSSINSSGGSTEYASSVKGRFTVSR | |
| | DNAKNMLYLQMDRLKLEDTAVYYCA | |
| | DEGNPSTTWYYEDQGTQVTVSSGGG | |
| | GSGGGGEVQLVESGGGLVQPGGSLR | |
| | LSCAASGFTFSTYAMNWVRQAPGKG | |
| | LEWVGRIRSKYNNYATYYADSVKDR | |
| | FTISRDDSKNSLYLQMNSLKTEDTA | |
| | VYYCARHGNFGNSYVSYFAYWGQGT | |
| | LVTVSSGGSGGSDIQMTQSPSSLSA | |
| | SVGDRVTITCRSSTGAVTTSNYANW | |
| | VQQKPGKAPKALIGGTNKRAPGVPS | |
| | RFSGSLIGDKATLTISSLQPEDFAT | |
| | YYCALWYSNLWVFGQGTKVEIKGGG | |
| | GSGGGGSGGGGSEVQLVESGGGLVQ | |
| | PGGSLRLSCAASGFTFSTYAMNWVR | |
| | QAPGKGLEWVGRIRSKYNNYATYYA | |
| | DSVKDRFTISRDDSKNSLYLQMNSL | |
| | KTEDTAVYYCARHGNFGNSYVSYFA | |
| | YWGQGTLVTVSSGGSGGSDIQMTQS | |
| | PSSLSASVGDRVTITCRSSTGAVTT | |
| | SNYANWVQQKPGKAPKALIGGTNKR | |
| | APGVPSRFSGSLIGDKATLTISSLQ | |
| | PEDFATYYCALWYSNLWVFGQGTKV | |
| | EIKAAAGSHHHHHH | |

### SCP 6

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: TVHH-TVHH-EVL-EVH-EVL-EVH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 57. A summary of the amino acid sequences of the domains of SCP6 are provided in Table 16.

TABLE 16
Summary of Sequences for SCP 6

| | Amino Acid Sequence | SEQ ID |
|---|---|---|
| Description | (N to C) | NO: |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L5 | GGGGSGGGG | 5 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L4 | GGGGSGGGG | 4 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L3 | GGSGGS | 3 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L2 | GGGGSGGGGSGGGGS | 2 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L1 | GGSGGS | 1 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L6 | AAAGS | 6 |
| Purification | HHHHHHH | 64 |
| Tag | | |
| Full length | QVQLQESGGGLVQPGGSLRLSCAAS | 57 |
| sequence of | GFTFGSYDMAWVRQAPGKGPEWVSS | |
| SCP 6 | INSSGGSTEYASSVKGRFTVSRDNA | |
| | KNMLYLQMDRLKLEDTAVYYCADEG | |
| | NPSTTWYYEDQGTQVTVSSGGGGSG | |
| | GGGQVQLQESGGGLVQPGGSLRLSC | |
| | AASGFTFGSYDMAWVRQAPGKGPEW | |
| | VSSINSSGGSTEYASSVKGRFTVSR | |
| | DNAKNMLYLQMDRLKLEDTAVYYCA | |
| | DEGNPSTTWYYEDQGTQVTVSSGGG | |
| | GSGGGGDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGSGGSEVQ | |
| | LVESGGGLVQPGGSLRLSCAASGFT | |
| | FSTYAMNWVRQAPGKGLEWVGRIRS | |
| | KYNNYATYYADSVKDRFTISRDDSK | |
| | NSLYLQMNSLKTEDTAVYYCARHGN | |
| | FGNSYVSYFAYWGQGTLVTVSSGGG | |
| | GSGGGGSGGGGSDIQMTQSPSSLSA | |
| | SVGDRVTITCRSSTGAVTTSNYANW | |
| | VQQKPGKAPKALIGGTNKRAPGVPS | |
| | RFSGSLIGDKATLTISSLQPEDFAT | |
| | YYCALWYSNLWVFGQGTKVEIKGGS | |
| | GGSEVQLVESGGGLVQPGGSLRLSC | |
| | AASGFTFSTYAMNWVRQAPGKGLEW | |
| | VGRIRSKYNNYATYYADSVKDRFTI | |
| | SRDDSKNSLYLQMNSLKTEDTAVYY | |
| | CARHGNFGNSYVSYFAYWGQGTLVT | |
| | VSSAAAGSHHHHHH | |

### SCP 7

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: TVHH-TVHH-EVH-EVL-EVH-EVL. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 58. A summary of the amino acid sequences of the domains of SCP 7 are provided in Table 17.

TABLE 17
Summary of Sequences for SCP 7

| | Amino Acid Sequence | SEQ ID |
|---|---|---|
| Description | (N to C) | NO: |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L5 | GGGGSGGGG | 5 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L4 | GGGGSGGGG | 4 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L3 | GGSGGS | 3 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L2 | GGGGSGGGGSGGGGS | 2 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L1 | GGSGGS | 1 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L6 | AAAGS | 6 |
| Purification | HHHHHH | 64 |
| Tag | | |
| Full length | QVQLQESGGGLVQAGGSLRLSCAAS | 58 |
| sequence of | GSIGSINFIFWYRQAPGKQREFVAR | |
| SCP 7 | ISRSGRSDYVESVKGRFTISRDNAK | |
| | NTVYLQMNNLKSEDTAVYYCALGNN | |
| | WGQGTQVTVSSGGGGSGGGGQVQLQ | |
| | ESGGGLVQAGGSLRLSCAASGSIGS | |
| | INFIFWYRQAPGKQREFVARISRSG | |
| | RSDYVESVKGRFTISRDNAKNTVYL | |
| | QMNNLKSEDTAVYYCALGNNWGQGT | |
| | QVTVSSGGGGSGGGGEVQLVESGGG | |
| | LVQPGGSLRLSCAASGFTFSTYAMN | |
| | WVRQAPGKGLEWVGRIRSKYNNYAT | |
| | YYADSVKDRFTISRDDSKNSLYLQM | |
| | NSLKTEDTAVYYCARHGNFGNSYVS | |
| | YFAYWGQGTLVTVSSGGSGGSDIQM | |
| | TQSPSSLSASVGDRVTITCRSSTGA | |
| | VTTSNYANWVQQKPGKAPKALIGGT | |
| | NKRAPGVPSRFSGSLIGDKATLTIS | |
| | SLOPEDFATYYCALWYSNLWVFGQG | |
| | TKVEIKGGGGSGGGGSGGGGSEVQL | |
| | VESGGGLVQPGGSLRLSCAASGFTF | |
| | STYAMNWVRQAPGKGLEWVGRIRSK | |
| | YNNYATYYADSVKDRFTISRDDSKN | |
| | SLYLQMNSLKTEDTAVYYCARHGNF | |
| | GNSYVSYFAYWGQGTLVTVSSGGSG | |
| | GSDIQMTQSPSSLSASVGDRVTITC | |
| | RSSTGAVTTSNYANWVQQKPGKAPK | |
| | ALIGGTNKRAPGVPSRFSGSLIGDK | |
| | ATLTISSLQPEDFATYYCALWYSNL | |
| | WVFGQGTKVEIKAAAGSHHHHHH | |

### SCP 8

In some embodiments, the single chain polypeptide comprises two target cell antigen interacting domains. In some embodiments, the variable domains and the two target cell antigen interacting domain are ordered according to the following N-terminal to C-terminal arrangement in which (-) is the linker: TVHH-TVHH-EVL-EVH-EVL-EVH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 59. A summary of the amino acid sequences of the domains of SCP 8 are provided in Table 18.

TABLE 18
Summary of Sequences for SCP 8

| | | SEQ |
|---|---|---|
| | Amino Acid Sequence | ID |
| Description | (N to C) | NO: |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L5 | GGGGSGGGG | 5 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L4 | GGGGSGGGG | 4 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L3 | GGSGGS | 3 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L2 | GGGGSGGGGSGGGGS | 2 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L1 | GGSGGS | 1 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L6 | AAAGS | 6 |
| Purification | HHHHHH | 64 |
| Tag | | |
| Full length | QVQLQESGGGLVQAGGSLRLSCAAS | 59 |
| sequence of | GSIGSINFIFWYRQAPGKQREFVAR | |
| SCP 8 | ISRSGRSDYVESVKGRFTISRDNAK | |
| | NTVYLQMNNLKSEDTAVYYCALGNN | |
| | WGQGTQVTVSSGGGGSGGGGQVQLQ | |
| | ESGGGLVQAGGSLRLSCAASGSIGS | |
| | INFIFWYRQAPGKQREFVARISRSG | |
| | RSDYVESVKGRFTISRDNAKNTVYL | |
| | QMNNLKSEDTAVYYCALGNNWGQGT | |
| | QVTVSSGGGGSGGGGDIQMTQSPSS | |
| | LSASVGDRVTITCRSSTGAVTTSNY | |
| | ANWVQQKPGKAPKALIGGTNKRAPG | |
| | VPSRFSGSLIGDKATLTISSLQPED | |
| | FATYYCALWYSNLWVFGQGTKVEIK | |
| | GGSGGSEVQLVESGGGLVQPGGSLR | |
| | LSCAASGFTFSTYAMNWVRQAPGKG | |
| | LEWVGRIRSKYNNYATYYADSVKDR | |
| | FTISRDDSKNSLYLQMNSLKTEDTA | |
| | VYYCARHGNFGNSYVSYFAYWGQGT | |
| | LVTVSSGGGGSGGGGSGGGGSDIQM | |
| | TQSPSSLSASVGDRVTITCRSSTGA | |
| | VTTSNYANWVQQKPGKAPKALIGGT | |
| | NKRAPGVPSRFSGSLIGDKATLTIS | |
| | SLQPEDFATYYCALWYSNLWVFGQG | |
| | TKVEIKGGSGGSEVQLVESGGGLVQ | |
| | PGGSLRLSCAASGFTFSTYAMNWVR | |
| | QAPGKGLEWVGRIRSKYNNYATYYA | |
| | DSVKDRFTISRDDSKNSLYLQMNSL | |
| | KTEDTAVYYCARHGNFGNSYVSYFA | |
| | YWGQGTLVTVSSAAAGSHHHHHH | |

### SCP 9

In some embodiments, the single chain polypeptide comprises one target cell antigen interacting domain. In some embodiments, the variable domains and the one target cell antigen interacting domain are ordered according to the following N-terminal to C-terminal arrangement in which (-) is the linker: EVH-EVL-EVH-EVL-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 60. A summary of the amino acid sequences of the domains of SCP 9 are provided in Table 19.

TABLE 19
Summary of Sequences for SCP 9

| | Amino Acid Sequence | SEQ ID |
|---|---|---|
| Description | (N to C) | NO: |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L1 | GGSGGS | 1 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L2 | GGGGSGGGGSGGGGS | 2 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L3 | GGSGGS | 3 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L4 | GGGGSGGGG | 4 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L6 | AAAGS | 6 |
| Purification | HHHHHH | 64 |
| Tag | | |
| Full length | EVQLVESGGGLVQPGGSLRLSCAAS | 60 |
| sequence of | GFTFSTYAMNWVRQAPGKGLEWVGR | |
| SCP 9 | IRSKYNNYATYYADSVKDRFTISRD | |
| | DSKNSLYLQMNSLKTEDTAVYYCAR | |
| | HGNFGNSYVSYFAYWGQGTLVTVSS | |
| | GGSGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGGGSGGGG | |
| | SGGGGSEVQLVESGGGLVQPGGSLR | |
| | LSCAASGFTFSTYAMNWVRQAPGKG | |
| | LEWVGRIRSKYNNYATYYADSVKDR | |
| | FTISRDDSKNSLYLQMNSLKTEDTA | |
| | VYYCARHGNFGNSYVSYFAYWGQGT | |
| | LVTVSSGGSGGSDIQMTQSPSSLSA | |
| | SVGDRVTITCRSSTGAVTTSNYANW | |
| | VQQKPGKAPKALIGGTNKRAPGVPS | |
| | RFSGSLIGDKATLTISSLQPEDFAT | |
| | YYCALWYSNLWVFGQGTKVEIKGGG | |
| | GSGGGGQVQLQESGGGLVQPGGSLR | |
| | LSCAASGFTFGSYDMAWVRQAPGKG | |
| | PEWVSSINSSGGSTEYASSVKGRFT | |
| | VSRDNAKNMLYLQMDRLKLEDTAVY | |
| | YCADEGNPSTTWYYEDQGTQVTVSS | |
| | AAAGSHHHHHH | |

### SCP 10

In some embodiments, the single chain polypeptide comprises one target cell antigen interacting domain. In some embodiments, the variable domains and the one target cell antigen interacting domain are ordered according to the following N-terminal to C-terminal arrangement in which (-) is the linker: EVL-EVH-EVL-EVH-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 61. A summary of the amino acid sequences of the domains of SCP 10 are provided in Table 20.

TABLE 20
Summary of Sequences for SCP 10

| | Amino Acid Sequence | SEQ ID |
|---|---|---|
| Description | (N to C) | NO: |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L1 | GGSGGS | 1 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L2 | GGGGSGGGGSGGGGS | 2 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L3 | GGSGGS | 3 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| LA | GGGGSGGGG | 4 |
| TVHH | QVQLQESGGGLVQPGGSLRLSCAAS | 32 |
| | GFTFGSYDMAWVRQAPGKGPEWVSS | |
| | INSSGGSTEYASSVKGRFTVSRDNA | |
| | KNMLYLQMDRLKLEDTAVYYCADEG | |
| | NPSTTWYYEDQGTQVTVSS | |
| L6 | AAAGS | 6 |
| Purification | HHHHIHIH | 64 |
| Tag | | |
| Full length | DIQMTQSPSSLSASVGDRVTITCRS | 61 |
| sequence of | STGAVTTSNYANWVQQKPGKAPKAL | |
| SCP 10 | IGGTNKRAPGVPSRFSGSLIGDKAT | |
| | LTISSLQPEDFATYYCALWYSNLWV | |
| | FGQGTKVEIKGGSGGSEVQLVESGG | |
| | GLVQPGGSLRLSCAASGFTFSTYAM | |
| | NWVRQAPGKGLEWVGRIRSKYNNYA | |
| | TYYADSVKDRFTISRDDSKNSLYLQ | |
| | MNSLKTEDTAVYYCARHGNFGNSYV | |
| | SYFAYWGQGTLVTVSSGGGGSGGGG | |
| | SGGGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGSGGSEVQ | |
| | LVESGGGLVQPGGSLRLSCAASGFT | |
| | FSTYAMNWVRQAPGKGLEWVGRIRS | |
| | KYNNYATYYADSVKDRFTISRDDSK | |
| | NSLYLQMNSLKTEDTAVYYCARHGN | |
| | FGNSYVSYFAYWGQGTLVTVSSGGG | |
| | GSGGGGQVQLQESGGGLVQPGGSLR | |
| | LSCAASGFTFGSYDMAWVRQAPGKG | |
| | PEWVSSINSSGGSTEYASSVKGRFT | |
| | VSRDNAKNMLYLQMDRLKLEDTAVY | |
| | YCADEGNPSTTWYYEDQGTQVTVSS | |
| | AAAGSHHHHHH | |

### SCP 11

In some embodiments, the single chain polypeptide comprises one target cell antigen interacting domain. In some embodiments, the variable domains and the one target cell antigen interacting domain are ordered according to the following N-terminal to C-terminal arrangement in which (-) is the linker: EVH-EVL-EVH-EVL-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 62. A summary of the amino acid sequences of the domains of SCP 11 are provided in Table 21.

TABLE 21
Summary of Sequences for SCP 11

| | | SEQ ID |
|---|---|---|
| Description | Amino Acid Sequence (N to C) | NO: |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L1 | GGSGGS | 1 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L2 | GGGGSGGGGSGGGGS | 2 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L3 | GGSGGS | 3 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L4 | GGGGSGGGG | 4 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L6 | AAAGS | 6 |
| Purification | HHHHHIH | 64 |
| Tag | | |
| Full length | EVQLVESGGGLVQPGGSLRLSCAAS | 62 |
| sequence of | GFTFSTYAMNWVRQAPGKGLEWVGR | |
| SCP 11 | IRSKYNNYATYYADSVKDRFTISRD | |
| | DSKNSLYLQMNSLKTEDTAVYYCAR | |
| | HGNFGNSYVSYFAYWGQGTLVTVSS | |
| | GGSGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGGGSGGGG | |
| | SGGGGSEVQLVESGGGLVQPGGSLR | |
| | LSCAASGFTFSTYAMNWVRQAPGKG | |
| | LEWVGRIRSKYNNYATYYADSVKDR | |
| | FTISRDDSKNSLYLQMNSLKTEDTA | |
| | VYYCARHGNFGNSYVSYFAYWGQGT | |
| | LVTVSSGGSGGSDIQMTQSPSSLSA | |
| | SVGDRVTITCRSSTGAVTTSNYANW | |
| | VQQKPGKAPKALIGGTNKRAPGVPS | |
| | RFSGSLIGDKATLTISSLQPEDFAT | |
| | YYCALWYSNLWVFGQGTKVEIKGGG | |
| | GSGGGGQVQLQESGGGLVQAGGSLR | |
| | LSCAASGSIGSINFIFWYRQAPGKQ | |
| | REFVARISRSGRSDYVESVKGRFTI | |
| | SRDNAKNTVYLQMNNLKSEDTAVYY | |
| | CALGNNWGQGTQVTVSSAAAGSHHH | |
| | HHH | |

### SCP 12

In some embodiments, the single chain polypeptide comprises one target cell antigen interacting domain. In some embodiments, the variable domains and the one target cell antigen interacting domain are ordered according to the following N-terminal to C-terminal arrangement in which (-) is the linker: EVL-EVH-EVL-EVH-TVHH. In some embodiments, EVH comprises a variable heavy chain of a single chain variable fragment that binds to CD3, and EVL comprises a variable light chain of a single chain variable fragment that binds to CD3. In some embodiments, TVHH comprises a VHH single domain antibody that binds to CD33. In some embodiments, the single chain polypeptide comprises an amino acid sequence according to SEQ ID NO: 63. A summary of the amino acid sequences of the domains of SCP 12 are provided in Table 22.

TABLE 22
Summary of Sequences for SCP 12

| | Amino Acid Sequence | SEQ ID |
|---|---|---|
| Description | (N to C) | NO: |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L1 | GGSGGS | 1 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L2 | GGGGSGGGGSGGGGS | 2 |
| EVL | DIQMTQSPSSLSASVGDRVTITCRS | 9 |
| | STGAVTTSNYANWVQQKPGKAPKAL | |
| | IGGTNKRAPGVPSRFSGSLIGDKAT | |
| | LTISSLQPEDFATYYCALWYSNLWV | |
| | FGQGTKVEIK | |
| L3 | GGSGGS | 3 |
| EVH | EVQLVESGGGLVQPGGSLRLSCAAS | 7 |
| | GFTFSTYAMNWVRQAPGKGLEWVGR | |
| | IRSKYNNYATYYADSVKDRFTISRD | |
| | DSKNSLYLQMNSLKTEDTAVYYCAR | |
| | HGNFGNSYVSYFAYWGQGTLVTVSS | |
| L4 | GGGGSGGGG | 4 |
| TVHH | QVQLQESGGGLVQAGGSLRLSCAAS | 33 |
| | GSIGSINFIFWYRQAPGKQREFVAR | |
| | ISRSGRSDYVESVKGRFTISRDNAK | |
| | NTVYLQMNNLKSEDTAVYYCALGNN | |
| | WGQGTQVTVSS | |
| L6 | AAAGS | 6 |
| Purification | HHHHHH | 64 |
| Tag | | |
| Full length | DIQMTQSPSSLSASVGDRVTITCRS | 63 |
| sequence of | STGAVTTSNYANWVQQKPGKAPKAL | |
| SCP 12 | IGGTNKRAPGVPSRFSGSLIGDKAT | |
| | LTISSLQPEDFATYYCALWYSNLWV | |
| | FGQGTKVEIKGGSGGSEVQLVESGG | |
| | GLVQPGGSLRLSCAASGFTFSTYAM | |
| | NWVRQAPGKGLEWVGRIRSKYNNYA | |
| | TYYADSVKDRFTISRDDSKNSLYLQ | |
| | MNSLKTEDTAVYYCARHGNFGNSYV | |
| | SYFAYWGQGTLVTVSSGGGGSGGGG | |
| | SGGGGSDIQMTQSPSSLSASVGDRV | |
| | TITCRSSTGAVTTSNYANWVQQKPG | |
| | KAPKALIGGTNKRAPGVPSRFSGSL | |
| | IGDKATLTISSLQPEDFATYYCALW | |
| | YSNLWVFGQGTKVEIKGGSGGSEVQ | |
| | LVESGGGLVQPGGSLRLSCAASGFT | |
| | FSTYAMNWVRQAPGKGLEWVGRIRS | |
| | KYNNYATYYADSVKDRFTISRDDSK | |
| | NSLYLQMNSLKTEDTAVYYCARHGN | |
| | FGNSYVSYFAYWGQGTLVTVSSGGG | |
| | GSGGGGQVQLQESGGGLVQAGGSLR | |
| | LSCAASGSIGSINFIFWYRQAPGKQ | |
| | REFVARISRSGRSDYVESVKGRFTI | |
| | SRDNAKNTVYLQMNNLKSEDTAVYY | |
| | CALGNNWGQGTQVTVSSAAAGSHHH | |
| | HHH | |

### Conserved Variants of CDR Sequences and Heavy and Light Chain Domains

In some embodiments, binding domains of the binding molecules comprise immunologically active homologues or variants of the CDR sequences described herein. Accordingly in some embodiments, a CDR sequence in a heavy or light chain domain that binds to a target antigen or effector cell is similar to, but not identical to, the amino acid sequence depicted in SEQ ID NOs: 7-63. In certain instances, a CDR variant sequence has a sequence identity of 99%, 98%, 97%, 96%, 95%, 94%, 93%, 92%, 91%, 90%, 89%, 88%, 87%, 86%, 85%, 84%, 83%, 82%, 81%, or 80% compared to the sequence of SEQ ID NOs: 13-19 and which is immunologically active.

In further instances, a CDR variant sequence incorporates 1, 2, 3, 4, or 5 conserved amino acid substitutions. Conservative substitutions include amino acid substitutions that substitute a given amino acid with another amino acid of similar characteristics and further include, among the aliphatic amino acids interchange of alanine, valine, leucine, and isoleucine; interchange of the hydroxyl residues serine and threonine, exchange of the acidic residues aspartate and glutamate, substitution between the amide residues asparagine and glutamine, exchange of the basic residues lysine and arginine, and replacements among the aromatic residues phenylalanine and tyrosine.

In yet further instances, a CDR variant sequence incorporates substitutions that enhance properties of the CDR such as increase in stability, resistance to proteases and/or binding affinities to target antigen or CD3.

In other instances, a CDR variant sequence is modified to change non-critical residues or residues in non-critical regions. Amino acids that are not critical can be identified by known methods, such as affinity maturation, CDR walking, site-directed mutagenesis, crystallization, nuclear magnetic resonance, photoaffinity labeling, or alanine-scanning mutagenesis.

In further embodiments, binding molecules comprise heavy and light chain domains that are immunologically active homologues or variants of heavy and light chain domain sequences provided herein. Accordingly, in some embodiments, a binding domain comprises a heavy or light chain domain sequence that is similar to, but not identical to, a binding domain provided herein. In certain instances, a variant heavy or light chain domain sequence has a sequence identity of 99%, 98%, 97%, 96%, 95%, 94%, 93%, 92%, 91%, 90%, 89%, 88%, 87%, 86%, 85%, 84%, 83%, 82%, 81%, or 80% compared to a sequence herein, and which is immunologically active.

In further embodiments, a variant heavy or light chain domain sequence incorporates 1, 2, 3, 4, or 5 conserved amino acid substitutions. Conservative substitutions include amino acid substitutions that substitute a given amino acid with another amino acid of similar characteristics and further include, among the aliphatic amino acids interchange of alanine, valine, leucine, and isoleucine; interchange of the hydroxyl residues serine and threonine, exchange of the acidic residues aspartate and glutamate, substitution between the amide residues asparagine and glutamine, exchange of the basic residues lysine and arginine, and replacements among the aromatic residues phenylalanine and tyrosine.

In further instances, a variant heavy or light chain domain sequence incorporates substitutions that enhance properties of the CDR such as increase in stability, resistance to proteases and/or binding affinities to an antigen.

In further instances, a variant heavy or light chain domain sequence is modified to change non-critical residues or residues in non-critical regions. Amino acids that are not critical can be identified by known methods, such as affinity maturation, CDR walking, site-directed mutagenesis, crystallization, nuclear magnetic resonance, photoaffinity labeling, or alanine-scanning mutagenesis.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Methods of Treatment

In certain embodiments, disclosed herein, are antibodies useful for the treatment of a cancer or tumor. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. For treating bacterial or viral disease treatment includes, but is not limited to reducing one or more symptoms associated with the viral or bacterial disease, such as reducing fever, nausea, diarrhea, vomiting, sore-throat, cough, runny-nose, and/or rash. Treatment of bacterial or viral disease can reduce overall levels of virus or bacteria in the body, reduce a period which an individual can infect others, or reduce overall disease or convalescence time. Treatment of autoimmune or inflammatory diseases includes but is not limited to reduction in total or self-antibody levels, or reduction in total or self-cellular immune responses. Treatment can also be associate with specific symptoms of autoimmune disease related to an overzealous antibody or cellular immune response. Treatment of fibrotic disease may reduce or slow the appearance of fibrotic tissue or the deposition of collagen in the tissue. Treatment of cardiovascular disease may increase indicia of cardiovascular health including reducing blood pressure, reducing atherosclerotic lesions, or increasing heart function as indicated by the ability to pump blood. It is understood by those of skill in the art that not all individuals will respond equally, or at all, to a treatment that is administered, nevertheless these individuals are considered to be treated.

In certain embodiments, the single chain polypeptides are for use in treating a viral infection. In certain embodiments, the single chain polypeptides are for use in treating a bacterial infection. In certain embodiments, the single chain polypeptides are for use in treating a solid tumor cancer. In certain embodiments, the single chain polypeptides are for use in treating a hematological cancer. In certain embodiments, the single chain polypeptides are for use in treating an inflammatory condition. In certain embodiments, the single chain polypeptides are for use in treating an autoimmune disease. In certain embodiments the single chain polypeptides are for use in treating a cardiovascular disease. In certain embodiments, the single chain polypeptides are for use in treating a fibrotic disease.

The single chain polypeptide molecules described herein are contemplated for use as a medicament. Administration is effected by different ways, e.g. by intravenous, intraperitoneal, subcutaneous, intramuscular, intralesional, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently.

An “effective dose” refers to amounts of the active ingredient that are sufficient to affect the course and the severity of the disease, leading to the reduction or remission of such pathology. For example, an “effective dose” useful for treating and/or preventing a CD33⁺ cancer such as AML may be determined using known methods. Maximum tolerated doses (MTD) and maximum response doses (MRD) can be determined via established animal and human experimental protocols as well as in the examples described herein.

In some embodiments, administration of a polypeptide herein is at a dose level determined and contemplated by a medical practitioner. In certain therapeutic applications, polypeptide is administered to a patient already suffering from a cancer, in an amount sufficient to cure or at least partially arrest the symptoms of the cancer. Amounts effective for this use depend on the severity and course of the cancer, previous therapy, the patient's health status, weight, and response to the drugs, and the judgment of the treating physician. Therapeutically effective amounts are optionally determined by methods including, but not limited to, a dose escalation clinical trial, such as described in the examples.

### Pharmaceutical Compositions

In some aspects, provided herein are pharmaceutical compositions comprising the single chain polypeptide molecule, a vector comprising the polynucleotide encoding the single chain polypeptide molecule or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents. In further aspects, the pharmaceutical compositions comprise excipients for sustained release, e.g. PLGA nanoparticles and the like. In further aspects, the pharmaceutical compositions are coated on a device for insertion into the body for sustained release at a particular site.

In certain embodiments, the single chain polypeptides of the current disclosure are included in a pharmaceutical composition comprising one or more pharmaceutically acceptable excipients, carriers, and diluents. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the single chain polypeptides of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

Also described herein are kits comprising one or more of the single chain polypeptides described herein in a suitable container and one or more additional components selected from: instructions for use; a diluent, an excipient, a carrier, and a device for administration.

In certain embodiments, described herein is a method of preparing a cancer treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### Protein Production

In one aspect provided herein comprise a single chain polypeptide. The polypeptide is, in certain instances, a single chain fusion protein, which is not branched. For example, a plurality of antigen binding domains are linked in the polypeptide.

In some embodiments, a polypeptide described herein is produced by expressing polynucleotides encoding the polypeptide. Therefore, another aspect is a polynucleotide, e.g. DNA or RNA, encoding a polypeptide herein.

The polynucleotide is constructed by known methods such as by combining the genes encoding at least two or three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. The polynucleotide encoding said peptide linkers may appropriately be a flexible linker (e.g., a Gly-Ser linker; (Gly₄Ser)ₙ wherein n is 1, 2, 3, 4, 5, or more; (Gly₃Ser)ₙ wherein n is 1, 2, 3, 4, 5, or more(Gly₃Ser)ₙ wherein n is 1, 2, 3, 4, 5, or more). Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods.

A variety of expression vector/host systems may be utilized to contain and express the polynucleotide encoding the polypeptide. Examples of expression vectors for expression in E. coli are pSKK (Le Gall et al., J Immunol Methods. (2004) 285(1):111-27) or pcDNA5 (Invitrogen) for expression in mammalian cells.

Thus, the polypeptide as described herein, in some embodiments, is produced by introducing a vector encoding the polypeptide as described above into a host cell and culturing said host cell under conditions whereby the polypeptide chains are expressed, may be isolated and, optionally, further purified.

In some aspects, the single chain polypeptide described herein has a modification. Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, drug conjugation, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In further embodiments, the multivalent antibody is modified with additional amino acids, such as a leader or secretory sequence or a sequence for purification of the polypeptide.

In some aspects, a single chain polypeptide molecule described herein comprises a half-life extension domain that extends half-life of the polypeptide. In some embodiments, the half-life extension domain is connected to the N-terminus of the single chain polypeptide molecule. In some embodiments, the half-life extension domain is connected to the C-terminus of the single chain polypeptide molecule. In some embodiments, the half-life extension domain replaces a binding domain, such as a VHH domain of a molecule herein. In some embodiments, the half-life extension domain replaces a binding domain, such as a VH and VL pair of an single chain polypeptide molecule herein. Such domains are contemplated to include but are not limited to HSA binding domains, pegylation, small molecules, and other half-life extension domains known in the art. Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma. Noncovalent association with albumin extends the elimination half-time of proteins. In some embodiments, the half-life extension domain is a domain that binds to HSA including but not limited to domains from a monoclonal antibody, a polyclonal antibody, a recombinant antibody, a human antibody, a humanized antibody, a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA.

### EMBODIMENTS LISTING

Embodiment 1 comprises a single chain polypeptide comprising variable domains connected by linkers that fold into a conformation such that the variable domains form two effector cell antigen interacting domains (E) wherein each effector cell antigen interacting domain comprises two variable domains of a single chain variable fragment (EV and EV), and one of the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain.

Embodiment 2 comprises the single chain polypeptide of embodiment 1, wherein the two variable domains of a single chain variable fragment (EV and EV) comprise a variable heavy chain of a single chain variable fragment (EVH) and a variable light chain of single chain variable fragment (EVL).

Embodiment 3 comprises the single chain polypeptide of embodiments 1 or 2, wherein the one of the effector cell antigen interacting domains that is connected to the one or more target cell antigen interacting domains is connected to one target cell antigen interacting domain.

Embodiment 4 comprises the single chain polypeptide of embodiment 3, wherein the one target cell antigen interacting domain is connected C-terminal to the one of the effector cell antigen interacting domains.

Embodiment 5 comprises the single chain polypeptide of embodiment 3, wherein the one target cell antigen interacting domains is connected N-terminal to the one of the effector cell antigen interacting domains.

Embodiment 6 comprises the single chain polypeptide of embodiments 1 or 2, wherein the one of the effector cell antigen interacting domains that is connected to the one or more target cell antigen interacting domains is connected to two target cell antigen interacting domains.

Embodiment 7 comprises the single chain polypeptide of embodiment 6, wherein the two-target cell antigen interacting domains are connected C-terminal to the one of the effector cell antigen interacting domains.

Embodiment 8 comprises the single chain polypeptide of embodiment 6, wherein the two-target cell antigen interacting domains are connected N-terminal to the one of the effector cell antigen interacting domains.

Embodiment 9 comprises the single chain polypeptide of embodiments 1 or 2, wherein the one of the effector cell antigen interacting domains that is connected to the one or more target cell antigen interacting domains is connected to three target cell antigen interacting domains.

Embodiment 10 comprises the single chain polypeptide of embodiment 9, wherein the three-target cell antigen interacting domains are connected C-terminal to the one of the effector cell antigen interacting domains.

Embodiment 11 comprises the single chain polypeptide of embodiment 9, wherein the three-target cell antigen interacting domains are connected N-terminal to the one of the effector cell antigen interacting domains.

Embodiment 12 comprises the single chain polypeptide of embodiments 1 or 2, wherein the one of the effector cell antigen interacting domains that is connected to the one or more target cell antigen interacting domains is connected to four target cell antigen interacting domains.

Embodiment 13 comprises the single chain polypeptide of embodiment 12, wherein the four-target cell antigen interacting domains are connected C-terminal to the one of the effector cell antigen interacting domains.

Embodiment 14 comprises the single chain polypeptide of embodiment 12, wherein the four-target cell antigen interacting domains are connected N-terminal to the one of the effector cell antigen interacting domains.

Embodiment 15 comprises the single chain polypeptide of embodiment 2, wherein the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to one TVHH.

Embodiment 16 comprises the single chain polypeptide of embodiment 2, wherein the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to two TVHH.

Embodiment 17 comprises the single chain polypeptide of embodiment 2, wherein the EVL of the one of the effector cell antigen interacting domains that is connected to the one or more target cell antigen interacting domains is connected to one TVHH, and the EVH of the one of the effector cell antigen interacting domains that is connected to the one or more target cell antigen interacting domains is connected to three TVHH.

Embodiment 18 comprises the single chain polypeptide of embodiment 2, wherein the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to two TVHH.

Embodiment 19 comprises the single chain polypeptide of embodiment 2, wherein the EVH of the one of the effector cell antigen interacting domains that is connected to the one or more target cell antigen interacting domains is connected to one TVHH, and the EVL of the one of the effector cell antigen interacting domains that is connected to the one or more target cell antigen interacting domains is connected to three TVHH.

Embodiment 20 comprises the single chain polypeptide of embodiment 2, wherein the EVH of the one of the effector cell antigen interacting domains that is connected to the one or more target cell antigen interacting domains is connected to two TVHH, and the EVL of the one of the effector cell antigen interacting domains that is connected to the one or more target cell antigen interacting domains is connected to two TVHH.

Embodiment 21 comprises the single chain polypeptide of embodiments 2-20, wherein the variable domains and the one or more target cell antigen interacting domains are ordered according to the following N-terminal to C-terminal arrangements in which (-) is the linker:

TVHH-EV-EV-EV-EV;
EV-EV-EV-EV-TVHH;
TVHH-TVHH-EV-EV-EV-EV;
EV-EV-EV-EV-TVHH-TVHH;
TVHH-TVHH-TVHH-EV-EV-EV-EV;
EV-EV-EV-EV-TVHH-TVHH-TVHH;
TVHH-TVHH-TVHH-TVHH-EV-EV-EV-EV;
EV-EV-EV-EV-TVHH-TVHH-TVHH-TVHH;
TVHH-EV-EV-EV-EV-TVHH;
TVHH-EV-EV-EV-EV-TVHH-TVHH;
TVHH-TVHH-EV-EV-EV-EV-TVHH;
TVHH -EV-EV-EV-EV-TVHH-TVHH-TVHH;
TVHH-TVHH-TVHH-EV-EV-EV-EV-TVHH;
or
TVHH-TVHH-EV-EV-EV-EV-TVHH-TVHH.

Embodiment 22 comprises the single chain polypeptide of embodiments 2-20, wherein the variable domains and the one or more target cell antigen interacting domains are ordered according to the following N-terminal to C-terminal arrangements in which (-) is the linker:

TVHH-EVL-EVH-EVL-EVH;
TVHH-EVH-EVL-EVH-EVL;
EVL-EVH-EVL-EVH-TVHH;
EVH-EVL-EVH-EVL-TVHH;
TVHH-TVHH-EVL-EVH-EVL-EVH;
TVHH-TVHH-EVH-EVL-EVH-EVL;
EVL-EVH-EVL-EVH-TVHH-TVHH;
EVH-EVL-EVH-EVL-TVHH-TVHH;
TVHH-TVHH-TVHH-EVL-EVH-EVL-EVH;
TVHH-TVHH-TVHH-EVH-EVL-EVH-EVL;
EVL-EVH-EVL-EVH-TVHH-TVHH-TVHH;
EVH-EVL-EVH-EVL-TVHH-TVHH-TVHH;
TVHH-TVHH-TVHH-TVHH-EVL-EVH-EVL-EVH;
TVHH-TVHH-TVHH-TVHH-EVH-EVL-EVH-EVL;
EVL-EVH-EVL-EVH-TVHH-TVHH-TVHH-TVHH;
EVH-EVL-EVH-EVL-TVHH-TVHH-TVHH-TVHH;
TVHH-EVL-EVH-EVL-EVH-TVHH;
TVHH-EVH-EVL-EVH-EVL-TVHH;
TVHH-EVL-EVH-EVL-EVH-TVHH-TVHH;
TVHH-EVH-EVL-EVH-EVL-TVHH-TVHH;
TVHH-TVHH-EVL-EVH-EVL-EVH-TVHH;
TVHH-TVHH-EVH-EVL-EVH-EVL-TVHH;
TVHH-EVL-EVH-EVL-EVH-TVHH-TVHH-TVHH;
TVHH-EVH-EVL-EVH-EVL-TVHH-TVHH-TVHH;
TVHH-TVHH-TVHH-EVL-EVH-EVL-EVH-TVHH;
TVHH-TVHH-TVHH-EVH-EVL-EVH-EVL-TVHH;
TVHH-TVHH-EVL-EVH-EVL-EVH-TVHH-TVHH;
or
TVHH-TVHH-EVH-EVL-EVH-EVL-TVHH-TVHH.

Embodiment 23 comprises the single chain polypeptide of embodiments 1-22, wherein the linkers that connect the two effector cell antigen interacting domains (E) to each other comprise internal linkers.

Embodiment 24 comprises the single chain polypeptide of embodiments 2-23, wherein the effector cell antigen interacting domain that is not connected directly to the one or more target cell antigen interacting domains comprises a terminal linker that connects EVL and EVH.

Embodiment 25 comprises the single chain polypeptide of embodiment 24, wherein each of the internal linkers is 6-8 amino acids in length.

Embodiment 26 comprises the single chain polypeptide of embodiment 25, wherein the terminal linker is 15 or 16 amino acids in length.

Embodiment 27 comprises the single chain polypeptide of embodiment 2, wherein both of the effector cell antigen interacting domains are connected directly to target cell antigen interacting domains.

Embodiment 28 comprises the single chain polypeptide of embodiment 27, wherein both of the effector cell antigen interacting domains are connected N-terminal and C-terminal to target cell antigen interacting domains.

Embodiment 29 comprises the single chain polypeptide of embodiments 27-28, wherein both of the effector cell antigen interacting domains comprise a terminal linker that connects EVL and EVH.

Embodiment 30 comprises the single chain polypeptide of embodiment 29, wherein the terminal linker is 15 or 16 amino acids in length.

Embodiment 31 comprises the single chain polypeptide of embodiments 27-30, wherein the variable domains and the target cell antigen interacting domains are ordered according to the following N-terminal to C-terminal arrangements in which (-) is the linker:

TVHH-EVH-EVL-TVHH-TVHH-EVH-EVL-TVHH;
TVHH-EVL-EVH-TVHH-TVHH-EVH-EVL-TVHH;
TVHH-EVH-EVL-TVHH-TVHH-EVL-EVH-TVHH;
and
TVHH-EVL-EVH-TVHH-TVHH-EVL-EVH-TVHH.

Embodiment 32 comprises the single chain polypeptide of embodiments 1-31, wherein each of the effector cell antigen interacting domains bind to an effector cell antigen.

Embodiment 33 comprises the single chain polypeptide of embodiment 32, wherein the effector cell antigen comprises CD3, CD16a, or Death Receptor 5 (DR5).

Embodiment 34 comprises the single chain polypeptide of embodiment 33, wherein the CD3 is CD3 delta, CD3 gamma, or CD3 epsilon.

Embodiment 35 comprises the single chain polypeptide of embodiment 32, wherein the effector cell antigen is on an effector cell.

Embodiment 36 comprises the single chain polypeptide of embodiment 35, wherein the effector cell comprises a T cell, NK cell, or a macrophage.

Embodiment 37 comprises the single chain polypeptide of embodiments 1-36, wherein the target cell antigen interacting domains that interact with a target cell (T) bind to a target cell antigen.

Embodiment 38 comprises the single chain polypeptide of embodiment 37, wherein the target cell antigen is clustered in a lipid raft when on a cell associated with a disease state and not clustered in a lipid raft when on a cell that is not associated with a disease state.

Embodiment 39 comprises the single chain polypeptide of embodiment 38, wherein the target cell antigen is clustered when on a cell associated with a disease state and not clustered when on a cell that is not associated with a disease state.

Embodiment 40 comprises the single chain polypeptide of embodiment 38, wherein the target cell antigen is a dimer, trimer, tetramer, or oligomer when on a cell associated with a disease state and a monomer or dimer when on a cell that is not associated with a disease state.

Embodiment 41 comprises the single chain polypeptide of embodiment 38, wherein the target cell antigen is part of a cell signaling complex when on a cell associated with a disease state and not part of the same cell signaling complex when on a cell that is not associated with a disease state.

Embodiment 42 comprises the single chain polypeptide of embodiment 38, wherein the target cell antigen is part of a cell signaling complex when on a cell associated with a disease state and not part of a cell signaling complex when on a cell that is not associated with a disease state.

Embodiment 43 comprises the single chain polypeptide of embodiments 37-40, wherein the target cell antigen comprises CD33, FAP, EGFR, HER2, or EpCAM.

Embodiment 44 comprises the single chain polypeptide of embodiments 38-41, wherein the cell associated with a disease state is a myeloid-cell, fibroblast, or cancer cell.

Embodiment 45 comprises a single chain polypeptide comprising variable domains connected by linkers that fold into a conformation such that the variable domains form one or more effector cell antigen interacting domains (E) wherein the effector cell antigen interacting domain comprises two variable domains of a single chain variable fragment (EV and EV), and the effector cell antigen interacting domains is connected directly to one or more target cell antigen interacting domains that interacts with a target cell wherein the target cell antigen interacting domain is a VHH single domain antibody (TVHH), wherein the single chain polypeptide does not comprise an antibody constant domain.

Embodiment 46 comprises a single chain polypeptide comprising two variable domains of a single chain variable fragment (EV and EV) connected by a linker that fold into a conformation to form an effector cell antigen interacting domain (E) wherein the effector cell antigen interacting domain is connected by one or more linkers to two or more target cell antigen interacting domains that interacts with a target cell wherein the target cell antigen interacting domain is a VHH single domain antibody (TVHH), wherein the single chain polypeptide does not comprise an antibody constant domain.

Embodiment 47 comprises the single chain polypeptide of embodiment 46, wherein the two variable domains of a single chain variable fragment (EV and EV) comprise a variable heavy chain of a single chain variable fragment (EVH) and a variable light chain of single chain variable fragment (EVL).

Embodiment 48 comprises the single chain polypeptide of embodiments 46-47, wherein the two or more target cell antigen interacting domains are connected C-terminal to the effector cell antigen interacting domain.

Embodiment 49 comprises the single chain polypeptide of embodiments 46-47, wherein the two or more target cell antigen interacting domains are connected N-terminal to the effector cell antigen interacting domain.

Embodiment 50 comprises the single chain polypeptide of embodiments 46-47, wherein the two or more target cell antigen interacting domains are connected N-terminal and C-terminal to the effector cell antigen interacting domain.

Embodiment 51 comprises the single chain polypeptide of embodiments 46-50, wherein the effector cell antigen interacting domain is connected to two target cell antigen interacting domains.

Embodiment 52 comprises the single chain polypeptide of embodiments 46-50, wherein the effector cell antigen interacting domain is connected to three target cell antigen interacting domains.

Embodiment 53 comprises the single chain polypeptide of embodiments 46-50, wherein the effector cell antigen interacting domain is connected to four target cell antigen interacting domains.

Embodiment 54 comprises the single chain polypeptide of embodiments 46-50, wherein the effector cell antigen interacting domain is connected to five target cell antigen interacting domains.

Embodiment 55 comprises the single chain polypeptide of embodiments 46-50, wherein the effector cell antigen interacting domain is connected to six target cell antigen interacting domains.

Embodiment 56 comprises the single chain polypeptide of embodiments 46-50, wherein the effector cell antigen interacting domain is connected to two target cell antigen interacting domains and the variable domains and the two target cell antigen interacting domains are ordered according to the following N-terminal to C-terminal arrangements in which (-) is the linker:

EVH-EVL-TVHH-TVHH;
EVL-EVH-TVHH-TVHH;
TVHH-TVHH-EVH-EVL;
TVHH-EVL-EVH-TVHH;
or
TVHH-EVH-EVL-TVHH.

Embodiment 57 comprises the single chain polypeptide of embodiments 46-50, wherein the effector cell antigen interacting domain is connected to three target cell antigen interacting domains and the variable domains and the three target cell antigen interacting domains are ordered according to the following N-terminal to C-terminal arrangements in which (-) is the linker:

TVHH-TVHH-TVHH-EVL-EVH;
EVH-EVL-TVHH-TVHH-TVHH;
EVL-EVH-TVHH-TVHH-TVHH;
TVHH-TVHH-TVHH-EVH-EVL;
TVHH-TVHH-EVL-EVH-TVHH;
TVHH-EVH-EVL-TVHH-TVHH;
TVHH-EVL-EVH-TVHH-TVHH;
or
TVHH-TVHH-EVH-EVL-TVHH.

Embodiment 58 comprises the single chain polypeptide of embodiments 46-50, wherein the effector cell antigen interacting domain is connected to four target cell antigen interacting domains and the variable domains and the four target cell antigen interacting domains are ordered according to the following N-terminal to C-terminal arrangements in which (-) is the linker:

TVHH-TVHH-TVHH-TVHH-EVL-EVH;
EVH-EVL-TVHH-TVHH-TVHH-TVHH;
EVL-EVH-TVHH-TVHH-TVHH-TVHH;
TVHH-TVHH-TVHH-TVHH-EVH-EVL;
TVHH-TVHH-TVHH-EVL-EVH-TVHH;
TVHH-EVH-EVL-TVHH-TVHH-TVHH;
TVHH-EVL-EVH-TVHH-TVHH-TVHH;
TVHH-TVHH-TVHH-EVH-EVL-TVHH;
TVHH-TVHH-EVL-EVH-TVHH-TVHH;
or
TVHH-TVHH-EVH-EVL-TVHH-TVHH.

Embodiment 59 comprises the single chain polypeptide of embodiments 46-50, wherein the effector cell antigen interacting domain is connected to five target cell antigen interacting domains and the variable domains and the five target cell antigen interacting domains are ordered according to the following N-terminal to C-terminal arrangements in which (-) is the linker:

TVHH-TVHH-TVHH-TVHH-TVHH-EVL-EVH;
EVH-EVL-TVHH-TVHH-TVHH-TVHH-TVHH;
EVL-EVH-TVHH-TVHH-TVHH-TVHH-TVHH;
TVHH-TVHH-TVHH-TVHH-TVHH-EVH-EVL;
TVHH-TVHH-TVHH-TVHH-EVL-EVH-TVHH;
TVHH-EVH-EVL-TVHH-TVHH-TVHH-TVHH;
TVHH-EVL-EVH-TVHH-TVHH-TVHH-TVHH;
TVHH-TVHH-TVHH-TVHH-EVH-EVL-TVHH;
TVHH-TVHH-TVHH-EVL-EVH-TVHH-TVHH;
TVHH-TVHH-EVH-EVL-TVHH-TVHH-TVHH;
TVHH-TVHH-EVL-EVH-TVHH-TVHH-TVHH;
or
TVHH-TVHH-TVHH-EVH-EVL-TVHH-TVHH.

Embodiment 60 comprises the single chain polypeptide of embodiments 46-50, wherein the effector cell antigen interacting domain is connected to six target cell antigen interacting domains and the variable domains and the six target cell antigen interacting domains are ordered according to the following N-terminal to C-terminal arrangements in which (-) is the linker:

TVHH-TVHH-TVHH-TVHH-TVHH-TVHH-EVL-EVH;
EVH-EVL-TVHH-TVHH-TVHH-TVHH-TVHH-TVHH;
EVL-EVH-TVHH-TVHH-TVHH-TVHH-TVHH-TVHH;
TVHH-TVHH-TVHH-TVHH-TVHH-TVHH-EVH-EVL;
TVHH-TVHH-TVHH-TVHH-TVHH-EVL-EVH-TVHH;
TVHH-EVH-EVL-TVHH-TVHH-TVHH-TVHH-TVHH;
TVHH-EVL-EVH-TVHH-TVHH-TVHH-TVHH-TVHH;
TVHH-TVHH-TVHH-TVHH-TVHH-EVH-EVL-TVHH;
TVHH-TVHH-TVHH-TVHH-EVL-EVH-TVHH-TVHH;
TVHH-TVHH-EVH-EVL-TVHH-TVHH-TVHH-TVHH;
TVHH-TVHH-EVL-EVH-TVHH-TVHH-TVHH-TVHH;
TVHH-TVHH-TVHH-TVHH-EVH-EVL-TVHH-TVHH;
TVHH-TVHH-TVHH-EVL-EVH-TVHH-TVHH-TVHH;
or
TVHH-TVHH-TVHH-EVH-EVL-TVHH-TVHH-TVHH.

Embodiment 61 comprises the single chain polypeptide of embodiments 46-50, wherein the effector cell antigen interacting domain is connected to six target cell antigen interacting domains and the variable domains and the six target cell antigen interacting domains are ordered according to the following N-terminal to C-terminal arrangements in which (-) is the linker:

TVHH-TVHH-TVHH-EVH-EVL-TVHH-TVHH-TVHH;
or
EVH-EVL-TVHH-TVHH-TVHH-TVHH-TVHH-TVHH.

Embodiment 62 comprises the single chain polypeptide of embodiments 47-61, wherein the effector cell antigen interacting domain comprises a terminal linker that connects EVL and EVH.

Embodiment 63 comprises the single chain polypeptide of embodiment 62, wherein the terminal linker is 15 or 16 amino acids in length.

Embodiment 64 comprises the single chain polypeptide of embodiments 46-63, wherein the one or more linkers that connects the two or more target cell antigen interacting domains comprises an internal linker.

Embodiment 65 comprises the single chain polypeptide of embodiment 64, wherein each of the internal linkers is 6-8 amino acids in length.

Embodiment 66 comprises the single chain polypeptide of embodiments 46-65, wherein the effector cell antigen interacting domains bind to an effector cell antigen.

Embodiment 67 comprises the single chain polypeptide of embodiment 66, wherein the effector cell antigen comprises CD3, CD16a, or Death Receptor 5 (DR5).

Embodiment 68 comprises the single chain polypeptide of embodiment 67, wherein the CD3 is CD3 delta, CD3 gamma, or CD3 epsilon.

Embodiment 69 comprises the single chain polypeptide of embodiment 68, wherein the effector cell antigen is on an effector cell.

Embodiment 70 comprises the single chain polypeptide of embodiment 69, wherein the effector cell comprises a T cell, NK cell, or a macrophage.

Embodiment 71 comprises the single chain polypeptide of embodiments 46-70, wherein the target cell antigen interacting domain that interacts with a target cell (T) binds to a target cell antigen.

Embodiment 72 comprises the single chain polypeptide of embodiment 71, wherein the target cell antigen is clustered in a lipid raft when on a cell associated with a disease state and not clustered in a lipid raft when on a cell that is not associated with a disease state.

Embodiment 73 comprises the single chain polypeptide of embodiment 71, wherein the target cell antigen is clustered when on a cell associated with a disease state and not clustered when on a cell that is not associated with a disease state.

Embodiment 74 comprises the single chain polypeptide of embodiment 71, wherein the target cell antigen is a dimer, trimer, tetramer, or oligomer when on a cell associated with a disease state and a monomer or dimer when on a cell that is not associated with a disease state.

Embodiment 75 comprises the single chain polypeptide of embodiment 71, wherein the target cell antigen is part of a cell signaling complex when on a cell associated with a disease state and not part of the same cell signaling complex when on a cell that is not associated with a disease state.

Embodiment 76 comprises the single chain polypeptide of embodiment 71, wherein the target cell antigen is part of a cell signaling complex when on a cell associated with a disease state and not part of a cell signaling complex when on a cell that is not associated with a disease state.

Embodiment 77 comprises the single chain polypeptide of embodiments 71-76, wherein the target cell antigen comprises CD33, FAP, EGFR, HER2, or EpCAM.

Embodiment 78 comprises the single chain polypeptide of embodiments 71-77, wherein the cell associated with a disease state is a myeloid-cell, fibroblast, or cancer cell.

Embodiment 79 comprises the single chain polypeptide of embodiments 46-78, wherein the single chain polypeptide has one effector cell antigen interacting domain.

Embodiment 80 comprises a pharmaceutical composition comprising: (a) the single chain polypeptide of any one of embodiments 1-79; and (b) a pharmaceutically acceptable excipient.

Embodiment 81 comprises an isolated recombinant nucleic acid encoding a single chain polypeptide according to any one of embodiments 1-78.

Embodiment 82 comprises a vector comprising an isolated nucleic acid according to embodiment 81.

Embodiment 83 comprises a host cell comprising an isolated nucleic acid of embodiment 81 or a vector according to embodiment 81.

Embodiment 84 comprises a method of treating a cancer comprising administering to a subject in need thereof a single chain polypeptide according to any one of embodiments 1-79.

Embodiment 85 comprises the method of embodiment 84, wherein the cancer is a solid tumor cancer.

Embodiment 86 comprises the method of embodiment 84, wherein the cancer is a hematological cancer.

Embodiment 87 comprises a method of treating an inflammatory disease or condition comprising administering to a subject in need thereof a single chain polypeptide according to any one of embodiments 1-78.

Embodiment 88 comprises a method of treating an autoimmune disease or condition comprising administering to a subject in need thereof a single chain polypeptide according to any one of embodiments 1-79.

Embodiment 89 comprises a method of treating a cardiovascular disease or condition comprising administering to a subject in need thereof a single chain polypeptide according to any one of embodiments 1-79.

Embodiment 90 comprises a method of treating a fibrotic disease or condition comprising administering to a subject in need thereof a single chain polypeptide according to any one of embodiments 1-79.

Embodiment 91 comprises a method of treating a bacterial infection comprising administering to the subject in need thereof a single chain polypeptide according to any one of embodiments 1-79.

Embodiment 92 comprises a method of treating a viral infection comprising administering to the subject in need thereof a single chain polypeptide according to any one of embodiments 1-79.

### Examples

The following illustrative examples are representative of embodiments of compositions and methods described herein and are not meant to be limiting in any way.

### Example 1

### Cloning of DNA Expression Constructs Encoding Single Chain Polypeptides Specific for CD3 and HER2.

The coding sequences of single chain polypeptides specific for CD3 and HER2 are generated.

For expression of single chain polypeptides in CHO cells, the coding sequence of each is cloned into a mammalian expression vector system. Expression constructs are designed to contain coding sequences for a C-terminal hexahistidine (6xHis)-tag to facilitate antibody secretion and purification, respectively.

### Expression of Single Chain Polypeptides in Stably Transfected CHO Cells

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing single chain polypeptides are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing single chain polypeptides are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Single chain polypeptides are produced in 10-day fed-batch cultures of stably transfected CHO cell lines by secretion into the cell culture supernatant. Cell culture supernatants are harvested after 10 days at culture viabilities of typically >75%. Samples are collected from production cultures every other day and cell density and viability is assessed. On day of harvest, cell culture supernatants are cleared by centrifugation and vacuum filtration before further use.

Protein expression titers and product integrity in cell culture supernatants are analyzed by SDS-PAGE.

### Purification of Single Chain Polypeptides

Single chain polypeptides are purified from CHO cell culture supernatants in a two-step procedure. His6-tagged constructs are subjected to Ni-NTA Superflow chromatography in a first step followed by preparative size exclusion chromatography (SEC) on Superdex 200 in a second step. Eluted single chain polypeptides are characterized with regard size and pooled. Pooled samples are buffer-exchanged and concentrated by ultrafiltration to a typical concentration of >1 mg/mL. Purity and homogeneity of final samples is assessed by SDS-PAGE under reducing and non-reducing conditions, followed by immunoblotting using an anti-His-Tag antibody as well as by analytical SEC, respectively. Purified proteins are stored at aliquots at −80° C. until use.

TABLE 23
Single Chain Polypeptide Constructs Specific for CD3 and HER2

| | Anti- | Anti- | Configuration | | SEQ ID |
|---|---|---|---|---|---|
| | HER2 | CD3 | (Expressed Y/N; | | NO: |
| | SEQ | SEQ | Yield in | | for Full |
| Construct | ID | ID | mg/purity | | Length |
| # | NO: | NOs: | in %) | Full length Sequence | Sequence |
| 1 | 44 | 75 (VH) | EVH-EVL-EVH- | EVQLVESGGGLVQPGGSLKLSCAAS | 79 |
| | | and 76 | EVL-TVHH- | GFTFNKYAINWVRQAPGKGLEWVAR | |
| | | (VL) | TVHH | IRSKYNNYATYYADQVKDRFTISRD | |
| | | | (Y/4.2/71) | DSKNTAYLQMNNLKTEDTAVYYCVR | |
| | | | | HANFGNSYISYWAYWGQGTLVTVSS | |
| | | | | GGGSGGGSQTVVTQEPSLTVSPGGT | |
| | | | | VTLTCASSTGAVTSGNYPNWVQQKP | |
| | | | | GQAPRGLIGGTKFLVPGTPARFSGS | |
| | | | | LLGGKAALTLSGVQPEDEAEYYCTL | |
| | | | | WYSNRWVFGGGTKLTVLGGGGSGGG | |
| | | | | GSGGGGSGGGGSEVQLVESGGGLVQ | |
| | | | | PGGSLKLSCAASGFTFNKYAINWVR | |
| | | | | QAPGKGLEWVARIRSKYNNYATYYA | |
| | | | | DQVKDRFTISRDDSKNTAYLQMNNL | |
| | | | | KTEDTAVYYCVRHANFGNSYISYWA | |
| | | | | YWGQGTLVTVSSGGGSGGGSQTVVT | |
| | | | | QEPSLTVSPGGTVTLTCASSTGAVT | |
| | | | | SGNYPNWVQQKPGQAPRGLIGGTKF | |
| | | | | LVPGTPARFSGSLLGGKAALTLSGV | |
| | | | | QPEDEAEYYCTLWYSNRWVFGGGTK | |
| | | | | LTVLGGGGSGGGGSGGGGSGGGGSE | |
| | | | | VQLVESGGSLVQPGGSLRLSCAASG | |
| | | | | FTFDDYAMSWVRQVPGKGLEWVSSI | |
| | | | | NWSGTHTDYADSVKGRFTISRNNAN | |
| | | | | NTLYLQMNSLKSEDTAVYYCAKNWR | |
| | | | | DAGTTWFEKSGSAGQGTQVTVSSGG | |
| | | | | GGSGGGGSGGGGSGGGGSEVQLVES | |
| | | | | GGSLVQPGGSLRLSCAASGFTFDDY | |
| | | | | AMSWVRQVPGKGLEWVSSINWSGTH | |
| | | | | TDYADSVKGRFTISRNNANNTLYLQ | |
| | | | | MNSLKSEDTAVYYCAKNWRDAGTTW | |
| | | | | FEKSGSAGQGTQVTVSSAAAGSHHH | |
| | | | | HHH | |
| 2 | 44 | 75 (VH) | EVL-EVH-EVL- | QTVVTQEPSLTVSPGGTVTLTCASS | 80 |
| | | and 76 | EVH-TVHH- | TGAVTSGNYPNWVQQKPGQAPRGLI | |
| | | (VL) | TVHH | GGTKFLVPGTPARFSGSLLGGKAAL | |
| | | | (Y/14/66.9) | TLSGVQPEDEAEYYCTLWYSNRWVF | |
| | | | | GGGTKLTVLGGGSGGGSEVQLVESG | |
| | | | | GGLVQPGGSLKLSCAASGFTFNKYA | |
| | | | | INWVRQAPGKGLEWVARIRSKYNNY | |
| | | | | ATYYADQVKDRFTISRDDSKNTAYL | |
| | | | | QMNNLKTEDTAVYYCVRHANFGNSY | |
| | | | | ISYWAYWGQGTLVTVSSGGGGSGGG | |
| | | | | GSGGGGSGGGGSQTVVTQEPSLTVS | |
| | | | | PGGTVTLTCASSTGAVTSGNYPNWV | |
| | | | | QQKPGQAPRGLIGGTKFLVPGTPAR | |
| | | | | FSGSLLGGKAALTLSGVQPEDEAEY | |
| | | | | YCTLWYSNRWVFGGGTKLTVLGGGS | |
| | | | | GGGSEVQLVESGGGLVQPGGSLKLS | |
| | | | | CAASGFTFNKYAINWVRQAPGKGLE | |
| | | | | WVARIRSKYNNYATYYADQVKDRFT | |
| | | | | ISRDDSKNTAYLQMNNLKTEDTAVY | |
| | | | | YCVRHANFGNSYISYWAYWGQGTLV | |
| | | | | TVSSGGGGSGGGGSGGGGSGGGGSE | |
| | | | | VQLVESGGSLVQPGGSLRLSCAASG | |
| | | | | FTFDDYAMSWVRQVPGKGLEWVSSI | |
| | | | | NWSGTHTDYADSVKGRFTISRNNAN | |
| | | | | NTLYLQMNSLKSEDTAVYYCAKNWR | |
| | | | | DAGTTWFEKSGSAGQGTQVTVSSGG | |
| | | | | GGSGGGGSGGGGSGGGGSEVQLVES | |
| | | | | GGSLVQPGGSLRLSCAASGFTFDDY | |
| | | | | AMSWVRQVPGKGLEWVSSINWSGTH | |
| | | | | TDYADSVKGRFTISRNNANNTLYLQ | |
| | | | | MNSLKSEDTAVYYCAKNWRDAGTTW | |
| | | | | FEKSGSAGQGTQVTVSSAAAGSHHH | |
| | | | | HHH | |
| 3 | 45 | 75 (VH) | EVH-EVL-EVH- | EVQLVESGGGLVQPGGSLKLSCAAS | 81 |
| | | and 76 | EVL-TVHH- | GFTFNKYAINWVRQAPGKGLEWVAR | |
| | | (VL) | TVHH | IRSKYNNYATYYADQVKDRFTISRD | |
| | | | (Y/7.4/70.2) | DSKNTAYLQMNNLKTEDTAVYYCVR | |
| | | | | HANFGNSYISYWAYWGQGTLVTVSS | |
| | | | | GGGSGGGSQTVVTQEPSLTVSPGGT | |
| | | | | VTLTCASSTGAVTSGNYPNWVQQKP | |
| | | | | GQAPRGLIGGTKFLVPGTPARFSGS | |
| | | | | LLGGKAALTLSGVQPEDEAEYYCTL | |
| | | | | WYSNRWVFGGGTKLTVLGGGGSGGG | |
| | | | | GSGGGGSGGGGSEVQLVESGGGLVQ | |
| | | | | PGGSLKLSCAASGFTFNKYAINWVR | |
| | | | | QAPGKGLEWVARIRSKYNNYATYYA | |
| | | | | DQVKDRFTISRDDSKNTAYLQMNNL | |
| | | | | KTEDTAVYYCVRHANFGNSYISYWA | |
| | | | | YWGQGTLVTVSSGGGSGGGSQTVVT | |
| | | | | QEPSLTVSPGGTVTLTCASSTGAVT | |
| | | | | SGNYPNWVQQKPGQAPRGLIGGTKF | |
| | | | | LVPGTPARFSGSLLGGKAALTLSGV | |
| | | | | QPEDEAEYYCTLWYSNRWVFGGGTK | |
| | | | | LTVLGGGGSGGGGSGGGGSGGGGSE | |
| | | | | VQLVESGGGLVQAGGSLRLSCAASG | |
| | | | | ITFSINTMGWYRQAPGKQRELVALI | |
| | | | | SSIGDTYYADSVKGRFTISRDNAKN | |
| | | | | TVYLQMNSLKPEDTAVYYCKRFRTM | |
| | | | | QGTDYWGQGTQVTVSSGGGGSGGGG | |
| | | | | SGGGGSGGGGSEVQLVESGGGLVQA | |
| | | | | GGSLRLSCAASGITFSINTMGWYRQ | |
| | | | | APGKQRELVALISSIGDTYYADSVK | |
| | | | | GRFTISRDNAKNTVYLQMNSLKPED | |
| | | | | TAVYYCKRFRTMQGTDYWGQGTQVT | |
| | | | | VSSAAAGSHHHHHH | |
| 4 | 45 75 (VH) | | EVL-EVH-EVL- | QTVVTQEPSLTVSPGGTVTLTCASS | 82 |
| | and 76 | | EVH-TVHH- | TGAVTSGNYPNWVQQKPGQAPRGLI | |
| | (VL) | | TVHH | GGTKFLVPGTPARFSGSLLGGKAAL | |
| | | | (Y/7.9/100.0) | TLSGVQPEDEAEYYCTLWYSNRWVF | |
| | | | | GGGTKLTVLGGGSGGGSEVQLVESG | |
| | | | | GGLVQPGGSLKLSCAASGFTFNKYA | |
| | | | | INWVRQAPGKGLEWVARIRSKYNNY | |
| | | | | ATYYADQVKDRFTISRDDSKNTAYL | |
| | | | | QMNNLKTEDTAVYYCVRHANFGNSY | |
| | | | | ISYWAYWGQGTLVTVSSGGGGSGGG | |
| | | | | GSGGGGSGGGGSQTVVTQEPSLTVS | |
| | | | | PGGTVTLTCASSTGAVTSGNYPNWV | |
| | | | | QQKPGQAPRGLIGGTKFLVPGTPAR | |
| | | | | FSGSLLGGKAALTLSGVQPEDEAEY | |
| | | | | YCTLWYSNRWVFGGGTKLTVLGGGS | |
| | | | | GGGSEVQLVESGGGLVQPGGSLKLS | |
| | | | | CAASGFTFNKYAINWVRQAPGKGLE | |
| | | | | WVARIRSKYNNYATYYADQVKDRFT | |
| | | | | ISRDDSKNTAYLQMNNLKTEDTAVY | |
| | | | | YCVRHANFGNSYISYWAYWGQGTLV | |
| | | | | TVSSGGGGSGGGGSGGGGSGGGGSE | |
| | | | | VQLVESGGGLVQAGGSLRLSCAASG | |
| | | | | ITFSINTMGWYRQAPGKQRELVALI | |
| | | | | SSIGDTYYADSVKGRFTISRDNAKN | |
| | | | | TVYLQMNSLKPEDTAVYYCKRFRTM | |
| | | | | QGTDYWGQGTQVTVSSGGGGSGGGG | |
| | | | | SGGGGSGGGGSEVQLVESGGGLVQA | |
| | | | | GGSLRLSCAASGITFSINTMGWYRQ | |
| | | | | APGKQRELVALISSIGDTYYADSVK | |
| | | | | GRFTISRDNAKNTVYLQMNSLKPED | |
| | | | | TAVYYCKRFRTMQGTDYWGQGTQVT | |
| | | | | VSSAAAGSHHHHHH | |
| 5 | 46 | 75 (VH) | EVH-EVL-EVH- | EVQLVESGGGLVQPGGSLKLSCAAS | 83 |
| | | and 76 | EVL-TVHH- | GFTFNKYAINWVRQAPGKGLEWVAR | |
| | | (VL) | TVHH | IRSKYNNYATYYADQVKDRFTISRD | |
| | | | (Y/3.7/100.0) | DSKNTAYLQMNNLKTEDTAVYYCVR | |
| | | | | HANFGNSYISYWAYWGQGTLVTVSS | |
| | | | | GGGSGGGSQTVVTQEPSLTVSPGGT | |
| | | | | VTLTCASSTGAVTSGNYPNWVQQKP | |
| | | | | GQAPRGLIGGTKFLVPGTPARFSGS | |
| | | | | LLGGKAALTLSGVQPEDEAEYYCTL | |
| | | | | WYSNRWVFGGGTKLTVLGGGGSGGG | |
| | | | | GSGGGGSGGGGSEVQLVESGGGLVQ | |
| | | | | PGGSLKLSCAASGFTFNKYAINWVR | |
| | | | | QAPGKGLEWVARIRSKYNNYATYYA | |
| | | | | DQVKDRFTISRDDSKNTAYLQMNNL | |
| | | | | KTEDTAVYYCVRHANFGNSYISYWA | |
| | | | | YWGQGTLVTVSSGGGSGGGSQTVVT | |
| | | | | QEPSLTVSPGGTVTLTCASSTGAVT | |
| | | | | SGNYPNWVQQKPGQAPRGLIGGTKF | |
| | | | | LVPGTPARFSGSLLGGKAALTLSGV | |
| | | | | QPEDEAEYYCTLWYSNRWVFGGGTK | |
| | | | | LTVLGGGGSGGGGSGGGGSGGGGSK | |
| | | | | VQLVESGGGLVQPGGSLRLSCAASG | |
| | | | | SIFGFNDMAWYRQAPGKQRELVALI | |
| | | | | SRVGVTSSADSVKGRFTISRVNAKD | |
| | | | | TVYLQMNSLKPEDTAVYYCYMDQRL | |
| | | | | DGSTLAYWGQGTQVTVSSGGGGSGG | |
| | | | | GGSGGGGSGGGGSKVQLVESGGGLV | |
| | | | | QPGGSLRLSCAASGSIFGFNDMAWY | |
| | | | | RQAPGKQRELVALISRVGVTSSADS | |
| | | | | VKGRFTISRVNAKDTVYLQMNSLKP | |
| | | | | EDTAVYYCYMDQRLDGSTLAYWGQG | |
| | | | | TQVTVSSAAAGSHHHHHH | |
| 6 | 46 | 75 (VH) | EVL-EVH-EVL- | QTVVTQEPSLTVSPGGTVTLTCASS | 84 |
| | | and 76 | EVH-TVHH- | TGAVTSGNYPNWVQQKPGQAPRGLI | |
| | | (VL) | TVHH | GGTKFLVPGTPARFSGSLLGGKAAL | |
| | | | (Y/6.9/100.0) | TLSGVQPEDEAEYYCTLWYSNRWVF | |
| | | | | GGGTKLTVLGGGSGGGSEVQLVESG | |
| | | | | GGLVQPGGSLKLSCAASGFTFNKYA | |
| | | | | INWVRQAPGKGLEWVARIRSKYNNY | |
| | | | | ATYYADQVKDRFTISRDDSKNTAYL | |
| | | | | QMNNLKTEDTAVYYCVRHANFGNSY | |
| | | | | ISYWAYWGQGTLVTVSSGGGGSGGG | |
| | | | | GSGGGGSGGGGSQTVVTQEPSLTVS | |
| | | | | PGGTVTLTCASSTGAVTSGNYPNWV | |
| | | | | QQKPGQAPRGLIGGTKFLVPGTPAR | |
| | | | | FSGSLLGGKAALTLSGVQPEDEAEY | |
| | | | | YCTLWYSNRWVFGGGTKLTVLGGGS | |
| | | | | GGGSEVQLVESGGGLVQPGGSLKLS | |
| | | | | CAASGFTFNKYAINWVRQAPGKGLE | |
| | | | | WVARIRSKYNNYATYYADQVKDRFT | |
| | | | | ISRDDSKNTAYLQMNNLKTEDTAVY | |
| | | | | YCVRHANFGNSYISYWAYWGQGTLV | |
| | | | | TVSSGGGGSGGGGSGGGGSGGGGSK | |
| | | | | VQLVESGGGLVQPGGSLRLSCAASG | |
| | | | | SIFGFNDMAWYRQAPGKQRELVALI | |
| | | | | SRVGVTSSADSVKGRFTISRVNAKD | |
| | | | | TVYLQMNSLKPEDTAVYYCYMDQRL | |
| | | | | DGSTLAYWGQGTQVTVSSGGGGSGG | |
| | | | | GGSGGGGSGGGGSKVQLVESGGGLV | |
| | | | | QPGGSLRLSCAASGSIFGFNDMAWY | |
| | | | | RQAPGKQRELVALISRVGVTSSADS | |
| | | | | VKGRFTISRVNAKDTVYLQMNSLKP | |
| | | | | EDTAVYYCYMDQRLDGSTLAYWGQG | |
| | | | | TQVTVSSAAAGSHHHHHH | |

### Example 2

SCP-CD3/HER2 Affinity Determination to huHER2

The aim of this Example is to determine by SPR, using Biacore 8K+, the affinity of SCP-CD3/HER2 construct samples to huHER2-His protein in multi cycle kinetics (MCK). Constructs were injected for binding in-solution to the immobilized huHER2-His protein. The equipment used was a Biacore 8K+ instrument, a CFJB614 CM5 Series S sensor chip (Cytivia, Cat nr. 29104992) immobilized with huHER2-His (AcroBiosystems; Cat. nr. HE2-H5225) by amine coupling at low density (≈1200 RUs) on flow channels 1-to-4 and at high density (≈5000 RUs) on flow channels 5-to-8. Construct samples that are engagers to huHER2 (Construct #1, 2, 3, 5 and 6) at 1, 10 and 100 nM were injected in flow cells 1 and 2 for 2 min at 30 μl/min. The control mAb: Human anti-HER2 (Trastuzumab) (R&D Systems; Cat. nr. MAB9589) at 5 nM was injected in flow cells 1 and 2 for 2 min at 30 μl/min. An irrelevant construct that is an engager to huCD33 at 1, 10 and 100 nM was injected in flow cells 1 and 2 for 2 min at 30 μl/min. The off-rate measurement was 300 seconds, baseline levels were restored with regeneration solution. The buffer used was 1×HBS-EP+pH 7.4. Data was analyzed using the multi cycle kinetics predefined evaluation method of the Biacore Insight Evaluation Software. Kinetic parameters were calculated using the 1:1 binding fitting model. Results are shown in Table 24.

TABLE 24
Summarizes SCP - CD3/HER2 Affinity Data to huHER2

| | | | | Kinetics | | | | |
|---|---|---|---|---|---|---|---|---|
| Construct | Immobilized | Immobilized | Rmax | Chi² | ka | kd | KD | KD |
| # | Ligand | Density | (RU) | (RU²) | (1/Ms) | (1/s) | (M) | (nM) |
| 1 | huHER2-His | Low | 508 | 382 | 4.1E+06 | 3.1E−05 | 7.7E−12 | 0.01 |
| 1 | huHER2-His | High | 1906 | 5500 | 5.3E+05 | <1.0E−06 | <1.9E−12 | <0.002 |
| 2 | huHER2-His | Low | 488 | 85 | 4.3E+06 | 7.9E−05 | 1.8E−11 | 0.02 |
| 2 | huHER2-His | High | 1878 | 201 | 7.6E+05 | 3.5E−05 | 4.6E−11 | 0.05 |
| 3 | huHER2-His | Low | 21 | 4 | 2.6E+08 | 2.9E+00 | 1.1E−08 | 11.0 |
| 3 | huHER2-His | High | 150 | 44 | 1.2E+09 | 9.1E+01 | 7.8E−08 | 78.0 |
| 5 | huHER2-His | Low | 573 | 66 | 2.1E+06 | 2.0E−04 | 9.5E−11 | 0.10 |
| 5 | huHER2-His | High | 1748 | 151 | 2.0E+06 | 1.6E−04 | 7.9E−11 | 0.079 |
| 6 | huHER2-His | Low | 489 | 38 | 2.0E+06 | 2.4E−04 | 1.2E−10 | 0.12 |
| 6 | huHER2-His | High | 1742 | 366 | 1.8E+06 | 1.5E−04 | 8.2E−11 | 0.082 |

### Example 3

### Characterization of Single Chain Polypeptides

The single chain polypeptide constructs according to Table 25 and Table 26 were made and purified according to the protocols outlined in Example 1.

TABLE 25
Single Chain Polypeptide Construct Summary

| | | Format | | | | | |
|---|---|---|---|---|---|---|---|
| | | N-terminal to | | | | | |
| | | C-terminal | | | | | |
| | | arrangement | | | | | |
| | | in which | | EVH | EVL | | |
| | | (-) | Effector | SEQ | SEQ | Target | TVHH |
| Construct | Configuration | is the | Cell | ID | ID | Cell | SEQ ID |
| # | (Figure) | linker | Antigen | NO: | NO: | Antigen | NO: |
| 7 | 4B | EVH-EVL-EVH- | CD3 | 75 | 76 | HER2 | 46 |
| | | EVL-TVHH | | | | | |
| 8 | 4A | EVL-EVH-EVL- | CD3 | 75 | 76 | HER2 | 46 |
| | | EVH-TVHH | | | | | |
| 9 | 1B | EVH-EVL-EVH- | CD3 | 75 | 76 | HER2 | 46 |
| | | EVL-TVHH- | | | | | |
| | | TVHH | | | | | |
| 10 | 1A | EVL-EVH-EVL- | CD3 | 75 | 76 | HER2 | 46 |
| | | EVH-TVHH- | | | | | |
| | | TVHH | | | | | |
| 11 | 5B | EVH-EVL-EVH- | CD3 | 75 | 76 | HER2 | 46 |
| | | EVL-TVHH- | | | | | |
| | | TVHH-TVHH | | | | | |
| 12 | 5A | EVL-EVH-EVL- | CD3 | 75 | 76 | HER2 | 46 |
| | | EVH-TVHH- | | | | | |
| | | TVHH-TVHH | | | | | |
| 13 | 2B | EVH-EVL-EVH- | CD3 | 75 | 76 | HER2 | 46 |
| | | EVL-TVHH- | | | | | |
| | | TVHH-TVHH- | | | | | |
| | | TVHH | | | | | |
| 14 | 2A | EVL-EVH-EVL- | CD3 | 75 | 76 | HER2 | 46 |
| | | EVH-TVHH- | | | | | |
| | | TVHH-TVHH- | | | | | |
| | | TVHH | | | | | |
| 15 | 6B | TVHH-EVH- | CD3 | 75 | 76 | HER2 | 46 |
| | | EVL-EVH-EVL- | | | | | |
| | | TVHH | | | | | |
| 16 | 6A | TVHH-EVL- | CD3 | 75 | 76 | HER2 | 46 |
| | | EVH-EVL-EVH- | | | | | |
| | | TVHH | | | | | |
| 17 | 3B | TVHH-TVHH- | CD3 | 75 | 76 | HER2 | 46 |
| | | EVH-EVL-EVH- | | | | | |
| | | EVL-TVHH- | | | | | |
| | | TVHH | | | | | |
| 18 | 3A | TVHH-TVHH- | CD3 | 75 | 76 | HER2 | 46 |
| | | EVL-EVH-EVL- | | | | | |
| | | EVH-TVHH- | | | | | |
| | | TVHH | | | | | |
| 19 | 1B | EVH-EVL-EVH- | CD3 | 75 | 76 | HER2 | 46 |
| | | EVL-TVHH- | | | | | |
| | | TVHH | | | | | |
| 20 | 1A | EVL-EVH-EVL- | CD3 | 75 | 76 | HER2 | 46 |
| | | EVH-TVHH- | | | | | |
| | | TVHH | | | | | |
| 21 | 1B | EVH-EVL-EVH- | CD3 | 75 | 76 | HER2 | 46 |
| | | EVL-TVHH- | | | | | |
| | | TVHH | | | | | |
| 22 | 1A | EVL-EVH-EVL- | CD3 | 75 | 76 | HER2 | 46 |
| | | EVH-TVHH- | | | | | |
| | | TVHH | | | | | |
| 23 | 1A | EVL-EVH-EVL- | CD3 | 75 | 76 | HER2 | 46 |
| | | EVH-TVHH- | | | | | |
| | | TVHH | | | | | |
| 24 | 1A | EVL-EVH-EVL- | CD3 | 75 | 76 | HER2 | 46 |
| | | EVH-TVHH- | | | | | |
| | | TVHH | | | | | |
| 25 | 1A | EVL-EVH-EVL- | CD3 | 75 | 76 | HER2 | 46 |
| | | EVH-TVHH- | | | | | |
| | | TVHH | | | | | |
| 26 | 4A | EVL-EVH-EVL- | CD3 | 75 | 76 | HER2 | 46 |
| | | EVH-TVHH | | | | | |
| 27 | 4A | EVL-EVH-EVL- | CD3 | 75 | 76 | HER2 | 46 |
| | | EVH-TVHH | | | | | |
| 28 | 4A | EVL-EVH-EVL- | CD3 | 75 | 76 | HER2 | 46 |
| | | EVH-TVHH | | | | | |
| 29 | 1B | EVH-EVL-EVH- | CD3 | 75 | 76 | EGFR- | 47 |
| | | EVL-TVHH- | | | | binder | |
| | | TVHH | | | | 2 | |
| 30 | 1A | EVL-EVH-EVL- | CD3 | 75 | 76 | EGFR- | 47 |
| | | EVH-TVHH- | | | | binder | |
| | | TVHH | | | | 2 | |
| 31 | 1B | EVH-EVL-EVH- | CD3 | 75 | 76 | EGFR- | 47 |
| | | EVL-TVHH- | | | | binder | |
| | | TVHH | | | | 2 | |
| 32 | 1A | EVL-EVH-EVL- | CD3 | 75 | 76 | EGFR- | 47 |
| | | EVH-TVHH- | | | | binder | |
| | | TVHH | | | | 2 | |
| 33 | 1A | EVL-EVH-EVL- | CD3 | 75 | 76 | EGFR- | 47 |
| | | EVH-TVHH- | | | | binder | |
| | | TVHH | | | | 2 | |
| 34 | 1A | EVL-EVH-EVL- | CD3 | 75 | 76 | EGFR- | 47 |
| | | EVH-TVHH- | | | | binder | |
| | | TVHH | | | | 2 | |
| 35 | 1A | EVL-EVH-EVL- | CD3 | 75 | 76 | EGFR- | 47 |
| | | EVH-TVHH- | | | | binder | |
| | | TVHH | | | | 2 | |
| 36 | 4A | EVL-EVH-EVL- | CD3 | 75 | 76 | EGFR- | 47 |
| | | EVH-TVHH | | | | binder | |
| | | | | | | 2 | |
| 37 | 4A | EVL-EVH-EVL- | CD3 | 75 | 76 | EGFR- | 47 |
| | | EVH-TVHH | | | | binder | |
| | | | | | | 2 | |
| 38 | 4A | EVL-EVH-EVL- | CD3 | 75 | 76 | EGFR- | 47 |
| | | EVH-TVHH | | | | binder | |
| | | | | | | 2 | |
| 39 | 1B | EVH-EVL-EVH- | CD3 | 75 | 76 | EGFR- | 47 |
| | | EVL-TVHH- | | | | binder | |
| | | TVHH | | | | 2 | |
| 40 | 1A | EVL-EVH-EVL- | CD3 | 75 | 76 | EGFR- | 47 |
| | | EVH-TVHH- | | | | binder | |
| | | TVHH | | | | 2 | |
| 41 | 1A | EVL-EVH-EVL- | CD3 | 77 | 78 | EGFR- | 47 |
| | | EVH-TVHH- | | | | binder | |
| | | TVHH | | | | 2 | |
| 42 | 1A | EVL-EVH-EVL- | CD3 | 86 | 87 | EGFR- | 47 |
| | | EVH-TVHH- | | | | binder | |
| | | TVHH | | | | 2 | |
| 43 | 2 scFv | na | CD3 | 75 | 76 | HER2 | 46 |
| 44 | 2 scFv | na | CD3 | 75 | 76 | HER2 | 46 |
| 45 | 2 scFv | na | CD3 | 75 | 76 | EGFR- | 47 |
| | | | | | | binder | |
| | | | | | | 2 | |
| 46 | 2 scFv | na | CD3 | 75 | 76 | EGFR- | 47 |
| | | | | | | binder | |
| | | | | | | 2 | |
| 47 | 1 scFv | na | CD3 | 75 | 76 | HER2 | 46 |
| 48 | 1 scFv | na | CD3 | 75 | 76 | HER2 | 46 |
| 49 | 1 scFv | na | CD3 | 75 | 76 | EGFR- | 47 |
| | | | | | | binder | |
| | | | | | | 2 | |
| 50 | 1 scFv | na | CD3 | 75 | 76 | EGFR- | 47 |
| | | | | | | binder | |
| | | | | | | 2 | |

TABLE 26
Sequences of Single Chain Polypeptides
of Table 25.

| Con- | | SEQ |
|---|---|---|
| struct | Amino Sequence | ID |
| # | (N-term to C-term) | NO |
| 7 | EVQLVESGGGLVQPGGSLKLSCAASGFTFNKYAIN | 88 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGSQTVVTQE | |
| | PSLTVSPGGTVTLTCASSTGAVTSGNYPNWVQQKP | |
| | GQAPRGLIGGTKFLVPGTPARFSGSLLGGKAALTL | |
| | SGVQPEDEAEYYCTLWYSNRWVFGGGTKLTVLGGG | |
| | SGGGSGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GSGGGSQTVVTQEPSLTVSPGGTVTLTCASSTGAV | |
| | TSGNYPNWVQQKPGQAPRGLIGGTKFLVPGTPARF | |
| | SGSLLGGKAALTLSGVQPEDEAEYYCTLWYSNRWV | |
| | FGGGTKLTVLGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSS | |
| 8 | QTVVTQEPSLTVSPGGTVTLTCASSTGAVTSGNYP | 89 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSS | |
| 9 | EVQLVESGGGLVQPGGSLKLSCAASGFTFNKYAIN | 90 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGSQTVVTQE | |
| | PSLTVSPGGTVTLTCASSTGAVTSGNYPNWVQQKP | |
| | GQAPRGLIGGTKFLVPGTPARFSGSLLGGKAALTL | |
| | SGVQPEDEAEYYCTLWYSNRWVFGGGTKLTVLGGG | |
| | SGGGSGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GSGGGSQTVVTQEPSLTVSPGGTVTLTCASSTGAV | |
| | TSGNYPNWVQQKPGQAPRGLIGGTKFLVPGTPARF | |
| | SGSLLGGKAALTLSGVQPEDEAEYYCTLWYSNRWV | |
| | FGGGTKLTVLGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| 10 | QTVVTQEPSLTVSPGGTVTLTCASSTGAVTSGNYP | 91 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| 11 | EVQLVESGGGLVQPGGSLKLSCAASGFTFNKYAIN | 92 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGSQTVVTQE | |
| | PSLTVSPGGTVTLTCASSTGAVTSGNYPNWVQQKP | |
| | GQAPRGLIGGTKFLVPGTPARFSGSLLGGKAALTL | |
| | SGVQPEDEAEYYCTLWYSNRWVFGGGTKLTVLGGG | |
| | SGGGSGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GSGGGSQTVVTQEPSLTVSPGGTVTLTCASSTGAV | |
| | TSGNYPNWVQQKPGQAPRGLIGGTKFLVPGTPARF | |
| | SGSLLGGKAALTLSGVQPEDEAEYYCTLWYSNRWV | |
| | FGGGTKLTVLGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| | GGGSGGGSGGGSGGGSKVQLVESGGGLVQPGGSLR | |
| | LSCAASGSIFGFNDMAWYRQAPGKQRELVALISRV | |
| | GVTSSADSVKGRFTISRVNAKDTVYLQMNSLKPED | |
| | TAVYYCYMDQRLDGSTLAYWGQGTQVTVSS | |
| 12 | QTVVTQEPSLTVSPGGTVTLTCASSTGAVTSGNYP | 93 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| | GGGSGGGSGGGSGGGSKVQLVESGGGLVQPGGSLR | |
| | LSCAASGSIFGFNDMAWYRQAPGKQRELVALISRV | |
| | GVTSSADSVKGRFTISRVNAKDTVYLQMNSLKPED | |
| | TAVYYCYMDQRLDGSTLAYWGQGTQVTVSS | |
| 13 | EVQLVESGGGLVQPGGSLKLSCAASGFTFNKYAIN | 94 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGSQTVVTQE | |
| | PSLTVSPGGTVTLTCASSTGAVTSGNYPNWVQQKP | |
| | GQAPRGLIGGTKFLVPGTPARFSGSLLGGKAALTL | |
| | SGVQPEDEAEYYCTLWYSNRWVFGGGTKLTVLGGG | |
| | SGGGSGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GSGGGSQTVVTQEPSLTVSPGGTVTLTCASSTGAV | |
| | TSGNYPNWVQQKPGQAPRGLIGGTKFLVPGTPARF | |
| | SGSLLGGKAALTLSGVQPEDEAEYYCTLWYSNRWV | |
| | FGGGTKLTVLGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| | GGGSGGGSGGGSGGGSKVQLVESGGGLVQPGGSLR | |
| | LSCAASGSIFGFNDMAWYRQAPGKQRELVALISRV | |
| | GVTSSADSVKGRFTISRVNAKDTVYLQMNSLKPED | |
| | TAVYYCYMDQRLDGSTLAYWGQGTQVTVSSGGGSG | |
| | GGSGGGSGGGSKVQLVESGGGLVQPGGSLRLSCAA | |
| | SGSIFGFNDMAWYRQAPGKQRELVALISRVGVTSS | |
| | ADSVKGRFTISRVNAKDTVYLQMNSLKPEDTAVYY | |
| | CYMDQRLDGSTLAYWGQGTQVTVSS | |
| 14 | QTVVTQEPSLTVSPGGTVTLTCASSTGAVTSGNYP | 95 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| | GGGSGGGSGGGSGGGSKVQLVESGGGLVQPGGSLR | |
| | LSCAASGSIFGFNDMAWYRQAPGKQRELVALISRV | |
| | GVTSSADSVKGRFTISRVNAKDTVYLQMNSLKPED | |
| | TAVYYCYMDQRLDGSTLAYWGQGTQVTVSSGGGSG | |
| | GGSGGGSGGGSKVQLVESGGGLVQPGGSLRLSCAA | |
| | SGSIFGFNDMAWYRQAPGKQRELVALISRVGVTSS | |
| | ADSVKGRFTISRVNAKDTVYLQMNSLKPEDTAVYY | |
| | CYMDQRLDGSTLAYWGQGTQVTVSS | |
| 15 | KVQLVESGGGLVQPGGSLRLSCAASGSIFGFNDMA | 96 |
| | WYRQAPGKQRELVALISRVGVTSSADSVKGRFTIS | |
| | RVNAKDTVYLQMNSLKPEDTAVYYCYMDQRLDGST | |
| | LAYWGQGTQVTVSSGGGSGGGSGGGSGGGSEVQLV | |
| | ESGGGLVQPGGSLKLSCAASGFTFNKYAINWVRQA | |
| | PGKGLEWVARIRSKYNNYATYYADQVKDRFTISRD | |
| | DSKNTAYLQMNNLKTEDTAVYYCVRHANFGNSYIS | |
| | YWAYWGQGTLVTVSSGGGSGGGSQTVVTQEPSLTV | |
| | SPGGTVTLTCASSTGAVTSGNYPNWVQQKPGQAPR | |
| | GLIGGTKFLVPGTPARFSGSLLGGKAALTLSGVQP | |
| | EDEAEYYCTLWYSNRWVFGGGTKLTVLGGGSGGGS | |
| | GGGSGGGSEVQLVESGGGLVQPGGSLKLSCAASGF | |
| | TFNKYAINWVRQAPGKGLEWVARIRSKYNNYATYY | |
| | ADQVKDRFTISRDDSKNTAYLQMNNLKTEDTAVYY | |
| | CVRHANFGNSYISYWAYWGQGTLVTVSSGGGSGGG | |
| | SQTVVTQEPSLTVSPGGTVTLTCASSTGAVTSGNY | |
| | PNWVQQKPGQAPRGLIGGTKFLVPGTPARFSGSLL | |
| | GGKAALTLSGVQPEDEAEYYCTLWYSNRWVFGGGT | |
| | KLTVLGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| 16 | KVQLVESGGGLVQPGGSLRLSCAASGSIFGFNDMA | 97 |
| | WYRQAPGKQRELVALISRVGVTSSADSVKGRFTIS | |
| | RVNAKDTVYLQMNSLKPEDTAVYYCYMDQRLDGST | |
| | LAYWGQGTQVTVSSGGGSGGGSGGGSGGGSQTVVT | |
| | QEPSLTVSPGGTVTLTCASSTGAVTSGNYPNWVQQ | |
| | KPGQAPRGLIGGTKFLVPGTPARFSGSLLGGKAAL | |
| | TLSGVQPEDEAEYYCTLWYSNRWVFGGGTKLTVLG | |
| | GGSGGGSEVQLVESGGGLVQPGGSLKLSCAASGFT | |
| | FNKYAINWVRQAPGKGLEWVARIRSKYNNYATYYA | |
| | DQVKDRFTISRDDSKNTAYLQMNNLKTEDTAVYYC | |
| | VRHANFGNSYISYWAYWGQGTLVTVSSGGGSGGGS | |
| | GGGSGGGSQTVVTQEPSLTVSPGGTVTLTCASSTG | |
| | AVTSGNYPNWVQQKPGQAPRGLIGGTKFLVPGTPA | |
| | RFSGSLLGGKAALTLSGVQPEDEAEYYCTLWYSNR | |
| | WVFGGGTKLTVLGGGSGGGSEVQLVESGGGLVQPG | |
| | GSLKLSCAASGFTFNKYAINWVRQAPGKGLEWVAR | |
| | IRSKYNNYATYYADQVKDRFTISRDDSKNTAYLQM | |
| | NNLKTEDTAVYYCVRHANFGNSYISYWAYWGQGTL | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| 17 | KVQLVESGGGLVQPGGSLRLSCAASGSIFGFNDMA | 98 |
| | WYRQAPGKQRELVALISRVGVTSSADSVKGRFTIS | |
| | RVNAKDTVYLQMNSLKPEDTAVYYCYMDQRLDGST | |
| | LAYWGQGTQVTVSSGGGSGGGSGGGSGGGSKVQLV | |
| | ESGGGLVQPGGSLRLSCAASGSIFGFNDMAWYRQA | |
| | PGKQRELVALISRVGVTSSADSVKGRFTISRVNAK | |
| | DTVYLQMNSLKPEDTAVYYCYMDQRLDGSTLAYWG | |
| | QGTQVTVSSGGGSGGGSGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSQTVVTQEPSLTVSPGGT | |
| | VTLTCASSTGAVTSGNYPNWVQQKPGQAPRGLIGG | |
| | TKFLVPGTPARFSGSLLGGKAALTLSGVQPEDEAE | |
| | YYCTLWYSNRWVFGGGTKLTVLGGGSGGGSGGGSG | |
| | GGSEVQLVESGGGLVQPGGSLKLSCAASGFTFNKY | |
| | AINWVRQAPGKGLEWVARIRSKYNNYATYYADQVK | |
| | DRFTISRDDSKNTAYLQMNNLKTEDTAVYYCVRHA | |
| | NFGNSYISYWAYWGQGTLVTVSSGGGSGGGSQTVV | |
| | TQEPSLTVSPGGTVTLTCASSTGAVTSGNYPNWVQ | |
| | QKPGQAPRGLIGGTKFLVPGTPARFSGSLLGGKAA | |
| | LTLSGVQPEDEAEYYCTLWYSNRWVFGGGTKLTVL | |
| | GGGSGGGSGGGSGGGSKVQLVESGGGLVQPGGSLR | |
| | LSCAASGSIFGFNDMAWYRQAPGKQRELVALISRV | |
| | GVTSSADSVKGRFTISRVNAKDTVYLQMNSLKPED | |
| | TAVYYCYMDQRLDGSTLAYWGQGTQVTVSSGGGSG | |
| | GGSGGGSGGGSKVQLVESGGGLVQPGGSLRLSCAA | |
| | SGSIFGFNDMAWYRQAPGKQRELVALISRVGVTSS | |
| | ADSVKGRFTISRVNAKDTVYLQMNSLKPEDTAVYY | |
| | CYMDQRLDGSTLAYWGQGTQVTVSS | |
| 18 | KVQLVESGGGLVQPGGSLRLSCAASGSIFGFNDMA | 99 |
| | WYRQAPGKQRELVALISRVGVTSSADSVKGRFTIS | |
| | RVNAKDTVYLQMNSLKPEDTAVYYCYMDQRLDGST | |
| | LAYWGQGTQVTVSSGGGSGGGSGGGSGGGSKVQLV | |
| | ESGGGLVQPGGSLRLSCAASGSIFGFNDMAWYRQA | |
| | PGKQRELVALISRVGVTSSADSVKGRFTISRVNAK | |
| | DTVYLQMNSLKPEDTAVYYCYMDQRLDGSTLAYWG | |
| | QGTQVTVSSGGGSGGGSGGGSGGGSQTVVTQEPSL | |
| | TVSPGGTVTLTCASSTGAVTSGNYPNWVQQKPGQA | |
| | PRGLIGGTKFLVPGTPARFSGSLLGGKAALTLSGV | |
| | QPEDEAEYYCTLWYSNRWVFGGGTKLTVLGGGSGG | |
| | GSEVQLVESGGGLVQPGGSLKLSCAASGFTFNKYA | |
| | INWVRQAPGKGLEWVARIRSKYNNYATYYADQVKD | |
| | RFTISRDDSKNTAYLQMNNLKTEDTAVYYCVRHAN | |
| | FGNSYISYWAYWGQGTLVTVSSGGGSGGGSGGGSG | |
| | GGSQTVVTQEPSLTVSPGGTVTLTCASSTGAVTSG | |
| | NYPNWVQQKPGQAPRGLIGGTKFLVPGTPARFSGS | |
| | LLGGKAALTLSGVQPEDEAEYYCTLWYSNRWVFGG | |
| | GTKLTVLGGGSGGGSEVQLVESGGGLVQPGGSLKL | |
| | SCAASGFTFNKYAINWVRQAPGKGLEWVARIRSKY | |
| | NNYATYYADQVKDRFTISRDDSKNTAYLQMNNLKT | |
| | EDTAVYYCVRHANFGNSYISYWAYWGQGTLVTVSS | |
| | GGGSGGGSGGGSGGGSKVQLVESGGGLVQPGGSLR | |
| | LSCAASGSIFGFNDMAWYRQAPGKQRELVALISRV | |
| | GVTSSADSVKGRFTISRVNAKDTVYLQMNSLKPED | |
| | TAVYYCYMDQRLDGSTLAYWGQGTQVTVSSGGGSG | |
| | GGSGGGSGGGSKVQLVESGGGLVQPGGSLRLSCAA | |
| | SGSIFGFNDMAWYRQAPGKQRELVALISRVGVTSS | |
| | ADSVKGRFTISRVNAKDTVYLQMNSLKPEDTAVYY | |
| | CYMDQRLDGSTLAYWGQGTQVTVSS | |
| 19 | EVQLVESGGGLVQPGGSLKLSCAASGFTFNKYAIN | 100 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGSGGSQTVVTQEPS | |
| | LTVSPGGTVTLTCASSTGAVTSGNYPNWVQQKPGQ | |
| | APRGLIGGTKFLVPGTPARFSGSLLGGKAALTLSG | |
| | VQPEDEAEYYCTLWYSNRWVFGGGTKLTVLGGGSG | |
| | GGSGGGSGGGSEVQLVESGGGLVQPGGSLKLSCAA | |
| | SGFTFNKYAINWVRQAPGKGLEWVARIRSKYNNYA | |
| | TYYADQVKDRFTISRDDSKNTAYLQMNNLKTEDTA | |
| | VYYCVRHANFGNSYISYWAYWGQGTLVTVSSGGSG | |
| | GSQTVVTQEPSLTVSPGGTVTLTCASSTGAVTSGN | |
| | YPNWVQQKPGQAPRGLIGGTKFLVPGTPARFSGSL | |
| | LGGKAALTLSGVQPEDEAEYYCTLWYSNRWVFGGG | |
| | TKLTVLGGGSGGGSGGGSGGGSKVQLVESGGGLVQ | |
| | PGGSLRLSCAASGSIFGFNDMAWYRQAPGKQRELV | |
| | ALISRVGVTSSADSVKGRFTISRVNAKDTVYLQMN | |
| | SLKPEDTAVYYCYMDQRLDGSTLAYWGQGTQVTVS | |
| | SGGGSGGGSGGGSGGGSKVQLVESGGGLVQPGGSL | |
| | RLSCAASGSIFGFNDMAWYRQAPGKQRELVALISR | |
| | VGVTSSADSVKGRFTISRVNAKDTVYLQMNSLKPE | |
| | DTAVYYCYMDQRLDGSTLAYWGQGTQVTVSS | |
| 20 | QTVVTQEPSLTVSPGGTVTLTCASSTGAVTSGNYP | 101 |
| | NWVQQKPGQAPRGLIGGTKFLVPGTPARFSGSLLG | |
| | GKAALTLSGVQPEDEAEYYCTLWYSNRWVFGGGTK | |
| | LTVLGGSGGSEVQLVESGGGLVQPGGSLKLSCAAS | |
| | GFTFNKYAINWVRQAPGKGLEWVARIRSKYNNYAT | |
| | YYADQVKDRFTISRDDSKNTAYLQMNNLKTEDTAV | |
| | YYCVRHANFGNSYISYWAYWGQGTLVTVSSGGGSG | |
| | GGSGGGSGGGSQTVVTQEPSLTVSPGGTVTLTCAS | |
| | STGAVTSGNYPNWVQQKPGQAPRGLIGGTKFLVPG | |
| | TPARFSGSLLGGKAALTLSGVQPEDEAEYYCTLWY | |
| | SNRWVFGGGTKLTVLGGSGGSEVQLVESGGGLVQP | |
| | GGSLKLSCAASGFTFNKYAINWVRQAPGKGLEWVA | |
| | RIRSKYNNYATYYADQVKDRFTISRDDSKNTAYLQ | |
| | MNNLKTEDTAVYYCVRHANFGNSYISYWAYWGQGT | |
| | LVTVSSGGGSGGGSGGGSGGGSKVQLVESGGGLVQ | |
| | PGGSLRLSCAASGSIFGFNDMAWYRQAPGKQRELV | |
| | ALISRVGVTSSADSVKGRFTISRVNAKDTVYLQMN | |
| | SLKPEDTAVYYCYMDQRLDGSTLAYWGQGTQVTVS | |
| | SGGGSGGGSGGGSGGGSKVQLVESGGGLVQPGGSL | |
| | RLSCAASGSIFGFNDMAWYRQAPGKQRELVALISR | |
| | VGVTSSADSVKGRFTISRVNAKDTVYLQMNSLKPE | |
| | DTAVYYCYMDQRLDGSTLAYWGQGTQVTVSS | |
| 21 | EVQLVESGGGLVQPGGSLKLSCAASGFTFNKYAIN | 102 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGQTVVTQEP | |
| | SLTVSPGGTVTLTCASSTGAVTSGNYPNWVQQKPG | |
| | QAPRGLIGGTKFLVPGTPARFSGSLLGGKAALTLS | |
| | GVQPEDEAEYYCTLWYSNRWVFGGGTKLTVLGGGS | |
| | GGGSGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGQTVVTQEPSLTVSPGGTVTLTCASSTGAVTS | |
| | GNYPNWVQQKPGQAPRGLIGGTKFLVPGTPARFSG | |
| | SLLGGKAALTLSGVQPEDEAEYYCTLWYSNRWVFG | |
| | GGTKLTVLGGGSGGGSGGGSGGGSKVQLVESGGGL | |
| | VQPGGSLRLSCAASGSIFGFNDMAWYRQAPGKQRE | |
| | LVALISRVGVTSSADSVKGRFTISRVNAKDTVYLQ | |
| | MNSLKPEDTAVYYCYMDQRLDGSTLAYWGQGTQVT | |
| | VSSGGGSGGGSGGGSGGGSKVQLVESGGGLVQPGG | |
| | SLRLSCAASGSIFGFNDMAWYRQAPGKQRELVALI | |
| | SRVGVTSSADSVKGRFTISRVNAKDTVYLQMNSLK | |
| | PEDTAVYYCYMDQRLDGSTLAYWGQGTQVTVSS | |
| 22 | QTVVTQEPSLTVSPGGTVTLTCASSTGAVTSGNYP | 103 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGEVQLVESGGGLVQPGGSLKLSCAA | |
| | SGFTFNKYAINWVRQAPGKGLEWVARIRSKYNNYA | |
| | TYYADQVKDRFTISRDDSKNTAYLQMNNLKTEDTA | |
| | VYYCVRHANFGNSYISYWAYWGQGTLVTVSSGGGS | |
| | GGGSGGGSGGGSQTVVTQEPSLTVSPGGTVTLTCA | |
| | SSTGAVTSGNYPNWVQQKPGQAPRGLIGGTKFLVP | |
| | GTPARFSGSLLGGKAALTLSGVQPEDEAEYYCTLW | |
| | YSNRWVFGGGTKLTVLGGGSGGGEVQLVESGGGLV | |
| | QPGGSLKLSCAASGFTFNKYAINWVRQAPGKGLEW | |
| | VARIRSKYNNYATYYADQVKDRFTISRDDSKNTAY | |
| | LQMNNLKTEDTAVYYCVRHANFGNSYISYWAYWGQ | |
| | GTLVTVSSGGGSGGGSGGGSGGGSKVQLVESGGGL | |
| | VQPGGSLRLSCAASGSIFGFNDMAWYRQAPGKQRE | |
| | LVALISRVGVTSSADSVKGRFTISRVNAKDTVYLQ | |
| | MNSLKPEDTAVYYCYMDQRLDGSTLAYWGQGTQVT | |
| | VSSGGGSGGGSGGGSGGGSKVQLVESGGGLVQPGG | |
| | SLRLSCAASGSIFGFNDMAWYRQAPGKQRELVALI | |
| | SRVGVTSSADSVKGRFTISRVNAKDTVYLQMNSLK | |
| | PEDTAVYYCYMDQRLDGSTLAYWGQGTQVTVSS | |
| 23 | QTVVTQEPSLTVSPGGTVTLTCASSTGAVTSGNYP | 104 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSKVQLVESGGGLVQPGGS | |
| | LRLSCAASGSIFGFNDMAWYRQAPGKQRELVALIS | |
| | RVGVTSSADSVKGRFTISRVNAKDTVYLQMNSLKP | |
| | EDTAVYYCYMDQRLDGSTLAYWGQGTQVTVSSGGG | |
| | SGGGSGGGSGGGSKVQLVESGGGLVQPGGSLRLSC | |
| | AASGSIFGFNDMAWYRQAPGKQRELVALISRVGVT | |
| | SSADSVKGRFTISRVNAKDTVYLQMNSLKPEDTAV | |
| | YYCYMDQRLDGSTLAYWGQGTQVTVSS | |
| 24 | QTVVTQEPSLTVSPGGTVTLTCASSTGAVTSGNYP | 105 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSKVQLVESGGGLVQ | |
| | PGGSLRLSCAASGSIFGFNDMAWYRQAPGKQRELV | |
| | ALISRVGVTSSADSVKGRFTISRVNAKDTVYLQMN | |
| | SLKPEDTAVYYCYMDQRLDGSTLAYWGQGTQVTVS | |
| | SGGGSGGGSGGGSGGGSKVQLVESGGGLVQPGGSL | |
| | RLSCAASGSIFGFNDMAWYRQAPGKQRELVALISR | |
| | VGVTSSADSVKGRFTISRVNAKDTVYLQMNSLKPE | |
| | DTAVYYCYMDQRLDGSTLAYWGQGTQVTVSS | |
| 25 | QTVVTQEPSLTVSPGGTVTLTCASSTGAVTSGNYP | 106 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSGGGSKVQLV | |
| | ESGGGLVQPGGSLRLSCAASGSIFGFNDMAWYRQA | |
| | PGKQRELVALISRVGVTSSADSVKGRFTISRVNAK | |
| | DTVYLQMNSLKPEDTAVYYCYMDQRLDGSTLAYWG | |
| | QGTQVTVSSGGGSGGGSGGGSGGGSKVQLVESGGG | |
| | LVQPGGSLRLSCAASGSIFGFNDMAWYRQAPGKQR | |
| | ELVALISRVGVTSSADSVKGRFTISRVNAKDTVYL | |
| | QMNSLKPEDTAVYYCYMDQRLDGSTLAYWGQGTQV | |
| | TVSS | |
| 26 | QTVVTQEPSLTVSPGGTVTLTCASSTGAVTSGNYP | 107 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSKVQLVESGGGLVQPGGS | |
| | LRLSCAASGSIFGFNDMAWYRQAPGKQRELVALIS | |
| | RVGVTSSADSVKGRFTISRVNAKDTVYLQMNSLKP | |
| | EDTAVYYCYMDQRLDGSTLAYWGQGTQVTVSS | |
| 27 | QTVVTQEPSLTVSPGGTVTLTCASSTGAVTSGNYP | 108 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSKVQLVESGGGLVQ | |
| | PGGSLRLSCAASGSIFGFNDMAWYRQAPGKQRELV | |
| | ALISRVGVTSSADSVKGRFTISRVNAKDTVYLQMN | |
| | SLKPEDTAVYYCYMDQRLDGSTLAYWGQGTQVTVS | |
| | S | |
| 28 | QTVVTQEPSLTVSPGGTVTLTCASSTGAVTSGNYP | 109 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSGGGSKVQLV | |
| | ESGGGLVQPGGSLRLSCAASGSIFGFNDMAWYRQA | |
| | PGKQRELVALISRVGVTSSADSVKGRFTISRVNAK | |
| | DTVYLQMNSLKPEDTAVYYCYMDQRLDGSTLAYWG | |
| | QGTQVTVSS | |
| 29 | EVQLVESGGGLVQPGGSLKLSCAASGFTFNKYAIN | 110 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGSGGSQTVVTQEPS | |
| | LTVSPGGTVTLTCASSTGAVTSGNYPNWVQQKPGQ | |
| | APRGLIGGTKFLVPGTPARFSGSLLGGKAALTLSG | |
| | VQPEDEAEYYCTLWYSNRWVFGGGTKLTVLGGGSG | |
| | GGSGGGSGGGSEVQLVESGGGLVQPGGSLKLSCAA | |
| | SGFTFNKYAINWVRQAPGKGLEWVARIRSKYNNYA | |
| | TYYADQVKDRFTISRDDSKNTAYLQMNNLKTEDTA | |
| | VYYCVRHANFGNSYISYWAYWGQGTLVTVSSGGSG | |
| | GSQTVVTQEPSLTVSPGGTVTLTCASSTGAVTSGN | |
| | YPNWVQQKPGQAPRGLIGGTKFLVPGTPARFSGSL | |
| | LGGKAALTLSGVQPEDEAEYYCTLWYSNRWVFGGG | |
| | TKLTVLGGGSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSSGGGSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSS | |
| 30 | QTVVTQEPSLTVSPGGTVTLTCASSTGAVTSGNYP | 111 |
| | NWVQQKPGQAPRGLIGGTKFLVPGTPARFSGSLLG | |
| | GKAALTLSGVQPEDEAEYYCTLWYSNRWVFGGGTK | |
| | LTVLGGSGGSEVQLVESGGGLVQPGGSLKLSCAAS | |
| | GFTFNKYAINWVRQAPGKGLEWVARIRSKYNNYAT | |
| | YYADQVKDRFTISRDDSKNTAYLQMNNLKTEDTAV | |
| | YYCVRHANFGNSYISYWAYWGQGTLVTVSSGGGSG | |
| | GGSGGGSGGGSQTVVTQEPSLTVSPGGTVTLTCAS | |
| | STGAVTSGNYPNWVQQKPGQAPRGLIGGTKFLVPG | |
| | TPARFSGSLLGGKAALTLSGVQPEDEAEYYCTLWY | |
| | SNRWVFGGGTKLTVLGGSGGSEVQLVESGGGLVQP | |
| | GGSLKLSCAASGFTFNKYAINWVRQAPGKGLEWVA | |
| | RIRSKYNNYATYYADQVKDRFTISRDDSKNTAYLQ | |
| | MNNLKTEDTAVYYCVRHANFGNSYISYWAYWGQGT | |
| | LVTVSSGGGSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSSGGGSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSS | |
| 31 | EVQLVESGGGLVQPGGSLKLSCAASGFTFNKYAIN | 112 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGQTVVTQEP | |
| | SLTVSPGGTVTLTCASSTGAVTSGNYPNWVQQKPG | |
| | QAPRGLIGGTKFLVPGTPARFSGSLLGGKAALTLS | |
| | GVQPEDEAEYYCTLWYSNRWVFGGGTKLTVLGGGS | |
| | GGGSGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGQTVVTQEPSLTVSPGGTVTLTCASSTGAVTS | |
| | GNYPNWVQQKPGQAPRGLIGGTKFLVPGTPARFSG | |
| | SLLGGKAALTLSGVQPEDEAEYYCTLWYSNRWVFG | |
| | GGTKLTVLGGGSGGGSGGGSGGGSQVKLEESGGGS | |
| | VQTGGSLRLTCAASGRTSRSYGMGWFRQAPGKERE | |
| | FVSGISWRGDSTGYADSVKGRFTISRDNAKNTVDL | |
| | QMNSLKPEDTAIYYCAAAAGSAWYGTLYEYDYWGQ | |
| | GTQVTVSSGGGSGGGSGGGSGGGSQVKLEESGGGS | |
| | VQTGGSLRLTCAASGRTSRSYGMGWFRQAPGKERE | |
| | FVSGISWRGDSTGYADSVKGRFTISRDNAKNTVDL | |
| | QMNSLKPEDTAIYYCAAAAGSAWYGTLYEYDYWGQ | |
| | GTQVTVSS | |
| 32 | QTVVTQEPSLTVSPGGTVTLTCASSTGAVTSGNYP | 113 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGEVQLVESGGGLVQPGGSLKLSCAA | |
| | SGFTFNKYAINWVRQAPGKGLEWVARIRSKYNNYA | |
| | TYYADQVKDRFTISRDDSKNTAYLQMNNLKTEDTA | |
| | VYYCVRHANFGNSYISYWAYWGQGTLVTVSSGGGS | |
| | GGGSGGGSGGGSQTVVTQEPSLTVSPGGTVTLTCA | |
| | SSTGAVTSGNYPNWVQQKPGQAPRGLIGGTKFLVP | |
| | GTPARFSGSLLGGKAALTLSGVQPEDEAEYYCTLW | |
| | YSNRWVFGGGTKLTVLGGGSGGGEVQLVESGGGLV | |
| | QPGGSLKLSCAASGFTFNKYAINWVRQAPGKGLEW | |
| | VARIRSKYNNYATYYADQVKDRFTISRDDSKNTAY | |
| | LQMNNLKTEDTAVYYCVRHANFGNSYISYWAYWGQ | |
| | GTLVTVSSGGGSGGGSGGGSGGGSQVKLEESGGGS | |
| | VQTGGSLRLTCAASGRTSRSYGMGWFRQAPGKERE | |
| | FVSGISWRGDSTGYADSVKGRFTISRDNAKNTVDL | |
| | QMNSLKPEDTAIYYCAAAAGSAWYGTLYEYDYWGQ | |
| | GTQVTVSSGGGSGGGSGGGSGGGSQVKLEESGGGS | |
| | VQTGGSLRLTCAASGRTSRSYGMGWFRQAPGKERE | |
| | FVSGISWRGDSTGYADSVKGRFTISRDNAKNTVDL | |
| | QMNSLKPEDTAIYYCAAAAGSAWYGTLYEYDYWGQ | |
| | GTQVTVSS | |
| 33 | QTVVTQEPSLTVSPGGTVTLTCASSTGAVTSGNYP | 114 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSQVKLEESGGGSVQTGGS | |
| | LRLTCAASGRTSRSYGMGWFRQAPGKEREFVSGIS | |
| | WRGDSTGYADSVKGRFTISRDNAKNTVDLQMNSLK | |
| | PEDTAIYYCAAAAGSAWYGTLYEYDYWGQGTQVTV | |
| | SSGGGSGGGSGGGSGGGSQVKLEESGGGSVQTGGS | |
| | LRLTCAASGRTSRSYGMGWFRQAPGKEREFVSGIS | |
| | WRGDSTGYADSVKGRFTISRDNAKNTVDLQMNSLK | |
| | PEDTAIYYCAAAAGSAWYGTLYEYDYWGQGTQVTV | |
| | SS | |
| 34 | QTVVTQEPSLTVSPGGTVTLTCASSTGAVTSGNYP | 115 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSSGGGSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSS | |
| 35 | QTVVTQEPSLTVSPGGTVTLTCASSTGAVTSGNYP | 116 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSGGGSQVKLE | |
| | ESGGGSVQTGGSLRLTCAASGRTSRSYGMGWFRQA | |
| | PGKEREFVSGISWRGDSTGYADSVKGRFTISRDNA | |
| | KNTVDLQMNSLKPEDTAIYYCAAAAGSAWYGTLYE | |
| | YDYWGQGTQVTVSSGGGSGGGSGGGSGGGSQVKLE | |
| | ESGGGSVQTGGSLRLTCAASGRTSRSYGMGWFRQA | |
| | PGKEREFVSGISWRGDSTGYADSVKGRFTISRDNA | |
| | KNTVDLQMNSLKPEDTAIYYCAAAAGSAWYGTLYE | |
| | YDYWGQGTQVTVSS | |
| 36 | QTVVTQEPSLTVSPGGTVTLTCASSTGAVTSGNYP | 117 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSQVKLEESGGGSVQTGGS | |
| | LRLTCAASGRTSRSYGMGWFRQAPGKEREFVSGIS | |
| | WRGDSTGYADSVKGRFTISRDNAKNTVDLQMNSLK | |
| | PEDTAIYYCAAAAGSAWYGTLYEYDYWGQGTQVTV | |
| | SS | |
| 37 | QTVVTQEPSLTVSPGGTVTLTCASSTGAVTSGNYP | 118 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSQVKLEESGGGSVQ | |
| | TGGSLRLTCAASGRTSRSYGMGWFRQAPGKEREFV | |
| | SGISWRGDSTGYADSVKGRFTISRDNAKNTVDLQM | |
| | NSLKPEDTAIYYCAAAAGSAWYGTLYEYDYWGQGT | |
| | QVTVSS | |
| 38 | QTVVTQEPSLTVSPGGTVTLTCASSTGAVTSGNYP | 119 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSGGGSQVKLE | |
| | ESGGGSVQTGGSLRLTCAASGRTSRSYGMGWFRQA | |
| | PGKEREFVSGISWRGDSTGYADSVKGRFTISRDNA | |
| | KNTVDLQMNSLKPEDTAIYYCAAAAGSAWYGTLYE | |
| | YDYWGQGTQVTVSS | |
| 39 | EVQLVESGGGLVQPGGSLKLSCAASGFTFNKYAIN | 120 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGSGGGSQTVVTQE | |
| | PSLTVSPGGTVTLTCASSTGAVTSGNYPNWVQQKP | |
| | GQAPRGLIGGTKFLVPGTPARFSGSLLGGKAALTL | |
| | SGVQPEDEAEYYCTLWYSNRWVFGGGTKLTVLGGG | |
| | SGGGSGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GSGGGSQTVVTQEPSLTVSPGGTVTLTCASSTGAV | |
| | TSGNYPNWVQQKPGQAPRGLIGGTKFLVPGTPARF | |
| | SGSLLGGKAALTLSGVQPEDEAEYYCTLWYSNRWV | |
| | FGGGTKLTVLGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSSGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSS | |
| 40 | QTVVTQEPSLTVSPGGTVTLTCASSTGAVTSGNYP | 121 |
| | NWVQQKPGQAPRGLIGGTKFLVPGTPARFSGSLLG | |
| | GKAALTLSGVQPEDEAEYYCTLWYSNRWVFGGGTK | |
| | LTVLGGGSGGGSEVQLVESGGGLVQPGGSLKLSCA | |
| | ASGFTFNKYAINWVRQAPGKGLEWVARIRSKYNNY | |
| | ATYYADQVKDRFTISRDDSKNTAYLQMNNLKTEDT | |
| | AVYYCVRHANFGNSYISYWAYWGQGTLVTVSSGGG | |
| | SGGGSGGGSGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGSGGGSEVQLVESGGG | |
| | LVQPGGSLKLSCAASGFTFNKYAINWVRQAPGKGL | |
| | EWVARIRSKYNNYATYYADQVKDRFTISRDDSKNT | |
| | AYLQMNNLKTEDTAVYYCVRHANFGNSYISYWAYW | |
| | GQGTLVTVSSGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSSGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSS | |
| 41 | EIVMTQSPATLSVSPGERATLSCRASQSVSSNLAW | 122 |
| | YQQKPGQAPRLLIYGASTRATGIPARFSGSGSGTE | |
| | FTLTISSLQSEDFAVYYCQQYNNWPWTFGQGTKVE | |
| | IKGGGSGGGSEVQLVESGGGLVQPGRSLRLSCAAS | |
| | GFTFDDYAMHWVRQAPGKGLEWVSGISWNSGSIGY | |
| | ADSVKGRFTISRDNAKNSLYLQMNSLRAEDTALYY | |
| | CAKDSRGYGDYRLGGAYWGQGTLVTVSSGGGSGGG | |
| | SGGGSGGGSEIVMTQSPATLSVSPGERATLSCRAS | |
| | QSVSSNLAWYQQKPGQAPRLLIYGASTRATGIPAR | |
| | FSGSGSGTEFTLTISSLQSEDFAVYYCQQYNNWPW | |
| | TFGQGTKVEIKGGGSGGGSEVQLVESGGGLVQPGR | |
| | SLRLSCAASGFTFDDYAMHWVRQAPGKGLEWVSGI | |
| | SWNSGSIGYADSVKGRFTISRDNAKNSLYLQMNSL | |
| | RAEDTALYYCAKDSRGYGDYRLGGAYWGQGTLVTV | |
| | SSGGGSGGGSGGGSGGGSQVKLEESGGGSVQTGGS | |
| | LRLTCAASGRTSRSYGMGWFRQAPGKEREFVSGIS | |
| | WRGDSTGYADSVKGRFTISRDNAKNTVDLQMNSLK | |
| | PEDTAIYYCAAAAGSAWYGTLYEYDYWGQGTQVTV | |
| | SSGGGSGGGSGGGSGGGSQVKLEESGGGSVQTGGS | |
| | LRLTCAASGRTSRSYGMGWFRQAPGKEREFVSGIS | |
| | WRGDSTGYADSVKGRFTISRDNAKNTVDLQMNSLK | |
| | PEDTAIYYCAAAAGSAWYGTLYEYDYWGQGTQVTV | |
| | SS | |
| 42 | QTVVTQEPSLTVSPGGTVTLTCGSSTGAVTSGNYP | 123 |
| | NWVQQKPGQAPRGLIGGTKFLAPGTPARFSGSLLG | |
| | GKAALTLSGVQPEDEAEYYCVLWYSNRWVFGGGTK | |
| | LTVLGGGSGGGSEVQLVESGGGLVQPGGSLKLSCA | |
| | ASGFTFNKYAMNWVRQAPGKGLEWVARIRSKYNNY | |
| | ATYYADSVKDRFTISRDDSKNTAYLQMNNLKTEDT | |
| | AVYYCVRHGNFGNSYISYWAYWGQGTLVTVSSGGG | |
| | SGGGSGGGSGGGSQTVVTQEPSLTVSPGGTVTLTC | |
| | GSSTGAVTSGNYPNWVQQKPGQAPRGLIGGTKFLA | |
| | PGTPARFSGSLLGGKAALTLSGVQPEDEAEYYCVL | |
| | WYSNRWVFGGGTKLTVLGGGSGGGSEVQLVESGGG | |
| | LVQPGGSLKLSCAASGFTFNKYAMNWVRQAPGKGL | |
| | EWVARIRSKYNNYATYYADSVKDRFTISRDDSKNT | |
| | AYLQMNNLKTEDTAVYYCVRHGNFGNSYISYWAYW | |
| | GQGTLVTVSSGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSSGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSS | |
| 43 | EVQLVESGGGLVQPGGSLKLSCAASGFTFNKYAIN | 124 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GGSGGGGSGGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSS | |
| 44 | EVQLVESGGGLVQPGGSLKLSCAASGFTFNKYAIN | 125 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GGSGGGGSGGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGGSGGGSKVQLVESGG | |
| | GLVQPGGSLRLSCAASGSIFGFNDMAWYRQAPGKQ | |
| | RELVALISRVGVTSSADSVKGRFTISRVNAKDTVY | |
| | LQMNSLKPEDTAVYYCYMDQRLDGSTLAYWGQGTQ | |
| | VTVSSGGGSGGGSGGGSGGGSKVQLVESGGGLVQP | |
| | GGSLRLSCAASGSIFGFNDMAWYRQAPGKQRELVA | |
| | LISRVGVTSSADSVKGRFTISRVNAKDTVYLQMNS | |
| | LKPEDTAVYYCYMDQRLDGSTLAYWGQGTQVTVSS | |
| 45 | EVQLVESGGGLVQPGGSLKLSCAASGFTFNKYAIN | 126 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GGSGGGGSGGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSS | |
| 46 | EVQLVESGGGLVQPGGSLKLSCAASGFTFNKYAIN | 127 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSEVQLVESGGGLVQPGGSLKLSC | |
| | AASGFTFNKYAINWVRQAPGKGLEWVARIRSKYNN | |
| | YATYYADQVKDRFTISRDDSKNTAYLQMNNLKTED | |
| | TAVYYCVRHANFGNSYISYWAYWGQGTLVTVSSGG | |
| | GGSGGGGSGGGGSQTVVTQEPSLTVSPGGTVTLTC | |
| | ASSTGAVTSGNYPNWVQQKPGQAPRGLIGGTKFLV | |
| | PGTPARFSGSLLGGKAALTLSGVQPEDEAEYYCTL | |
| | WYSNRWVFGGGTKLTVLGGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSSGGGSGGGSGGGSGGGSQVKLEESGG | |
| | GSVQTGGSLRLTCAASGRTSRSYGMGWFRQAPGKE | |
| | REFVSGISWRGDSTGYADSVKGRFTISRDNAKNTV | |
| | DLQMNSLKPEDTAIYYCAAAAGSAWYGTLYEYDYW | |
| | GQGTQVTVSS | |
| 47 | EVQLVESGGGLVQPGGSLKLSCAASGFTFNKYAIN | 128 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSKVQLVESGGGLVQPGGSLRLSC | |
| | AASGSIFGFNDMAWYRQAPGKQRELVALISRVGVT | |
| | SSADSVKGRFTISRVNAKDTVYLQMNSLKPEDTAV | |
| | YYCYMDQRLDGSTLAYWGQGTQVTVSS | |
| 48 | EVQLVESGGGLVQPGGSLKLSCAASGFTFNKYAIN | 129 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSKVQLVESGGGLVQPGGSLRLSC | |
| | AASGSIFGFNDMAWYRQAPGKQRELVALISRVGVT | |
| | SSADSVKGRFTISRVNAKDTVYLQMNSLKPEDTAV | |
| | YYCYMDQRLDGSTLAYWGQGTQVTVSSGGGSGGGS | |
| | GGGSGGGSKVQLVESGGGLVQPGGSLRLSCAASGS | |
| | IFGFNDMAWYRQAPGKQRELVALISRVGVTSSADS | |
| | VKGRFTISRVNAKDTVYLQMNSLKPEDTAVYYCYM | |
| | DQRLDGSTLAYWGQGTQVTVSS | |
| 49 | EVQLVESGGGLVQPGGSLKLSCAASGFTFNKYAIN | 130 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSQVKLEESGGGSVQTGGSLRLTC | |
| | AASGRTSRSYGMGWFRQAPGKEREFVSGISWRGDS | |
| | TGYADSVKGRFTISRDNAKNTVDLQMNSLKPEDTA | |
| | IYYCAAAAGSAWYGTLYEYDYWGQGTQVTVSS | |
| 50 | EVQLVESGGGLVQPGGSLKLSCAASGFTFNKYAIN | 131 |
| | WVRQAPGKGLEWVARIRSKYNNYATYYADQVKDRF | |
| | TISRDDSKNTAYLQMNNLKTEDTAVYYCVRHANFG | |
| | NSYISYWAYWGQGTLVTVSSGGGGSGGGGSGGGGS | |
| | QTVVTQEPSLTVSPGGTVTLTCASSTGAVTSGNYP | |
| | NWVQQKPGQAPRGLIGGTKFLVPGTPARFSGSLLG | |
| | GKAALTLSGVQPEDEAEYYCTLWYSNRWVFGGGTK | |
| | LTVLGGGGSGGGSQVKLEESGGGSVQTGGSLRLTC | |
| | AASGRTSRSYGMGWFRQAPGKEREFVSGISWRGDS | |
| | TGYADSVKGRFTISRDNAKNTVDLQMNSLKPEDTA | |
| | IYYCAAAAGSAWYGTLYEYDYWGQGTQVTVSSGGG | |
| | SGGGSGGGSGGGSQVKLEESGGGSVQTGGSLRLTC | |
| | AASGRTSRSYGMGWFRQAPGKEREFVSGISWRGDS | |
| | TGYADSVKGRFTISRDNAKNTVDLQMNSLKPEDTA | |
| | IYYCAAAAGSAWYGTLYEYDYWGQGTQVTVSS | |

### SCP-CD3/HER2 T Cell Cytotoxicity Assay

T cell killing activity for single chain polypeptides was assessed on MCF-7 and SKBR3 cancer cell lines. T cells were negatively selected from PBMCs (StemCells; Cat. nr. 70025.1, Lot nr. 220370304C, Donor ID RG2512 (Donor 6). The number of cells to use was adjusted to obtain a T cell to cancer cell (effector to target, E:T) ratio of 10:1 (1.0E+05: 1.0E+04). T cells were stained with CellTrace Violet (Invitrogen, Cat. nr. C34557), added to a flat-bottom 96-well plate and incubated in a 5% CO2, 37° C. humidified incubator for 48 hours. The single chain polypeptides were diluted in eleven ten-fold serial dilutions starting at 30 nM in complete cell culture medium. A negative control was used at 20 nM in PBMCs, SKBR3 or MFC-7 cell lines alone. Additional controls included unstained cancer and T cells, cancer and co-cultured cells alone incubated with top concentration of constructs. SKBR3 and MCF-7 cells were collected to be used in CellTiter-Glo® Luminescent Cell Viability Assay (Promega; Cat. nr. G7571). After 48 hours of incubation, SKBR3 or MCF7 cells were transferred to an opaque plate, incubated for 10 minutes at ambient temperature with a mixture of complete cell culture medium and CellTiter-Glo® reagent (Promega; Cat. nr. G7571). Cell viability was assessed by luminescence. Controls were unstained cells and CellTiter-Glo® reagent only. Luminescence was analyzed in Pherastar equipment. T cells were recovered to be used in cell activation assay (CD25 and CD69 staining). Supermatant containing T-cell conditioned media was frozen for further cytotoxic/activation assays. Buffers that were used included the following: complete cell culture medium: RPMI-1640 (Gibco; Cat. nr. A10491-01) supplemented with 10% FBS (Gibco; Cat. nr. 10270-106) and 1×Penicilin-Streptomycin (Gibco; Cat. nr. 15140122) and FACS Buffer: 1×PBS/0.5% HI-FBS (Heat-inactivated Fetal Bovine Serum)/1:1000 EDTA.

### SCP-CD3/HER2 T Cell Activation Assay

T cell activation for single chain polypeptides was assessed on MCF-7 and SKBR3 cancer cell lines. T cell activation was measured at 48 h by assessing the upregulation of T cell activation markers (CD25 and CD69) by FACS. After 48 h incubation, T cells were collected and labelled with CD25 and CD69 markers. A 1:1 detection mix of mouse anti-huCD25-PE (BD Biosciences; Cat. nr. 567214) at a final concentration of 2 μg/ml, and mouse anti-huCD69-APC (BD Biosciences; Cat. nr. 560711) diluted 1:100 was prepared in FACS buffer, 30 min on ice, protected from light. Controls used included unstained cells, PE detection only, APC detection only and double detection controls. The analysis was performed in IntelliCyt® iQue Screener PLUS. CD25 and CD69 expression were read, respectively, on BL2 (PE) and RL1 (APC) channels on the single cell population. Inside single cell gate, the % of double positive (CD25+CD69+) cells was determined. Assay buffers include FACS Buffer: 1×PBS/0.5% HI-FBS (Heat-inactivated Fetal Bovine Serum)/1:1000 EDTA.

### T Cell Proliferation Assay

T cell proliferation of T cells was assessed via Cell Trace Violet dye staining. Cell proliferation was evaluated 48 h after co-culture with SKBR3 and MCF7 cancer cell lines by FACS. T cells were incubated for 20 minutes at 37° C. incubator with Cell Trace violet staining solution, diluted in 1×PBS at 1 μM. Cell Trace violet staining was assessed by FACS at 0 hours and after 48 hours incubation with cancer cell lines and HER2 engagers. The plates were placed in a 5% CO2, 37° C. humidified incubator for 48 hours in co-culture with SKBR3 and MCF7 cancer cell lines. The control used was unstained T cells. Cells were acquired in the Intellicyt® iQue Screener PLUS and analysis performed in Intellicyt Forecyt Software. Cell Trace violet expression was read VL1 (Pacific Blue) channel on the single cell population. Sequence of H. pylori BabA Nb14/CD3 binder (SEQ ID NO: 85):

QVQLQESGGGLVQPGGSLRLSCAASGSIYSLIAMGWYRQAPGK
EHELVATISSGSTTYYADSVKGRFTISRDNAKNTLYLQMNSLK
PEDTAMYYCAAYSDRLTDCSNCEADYWGQGTQVTVSH

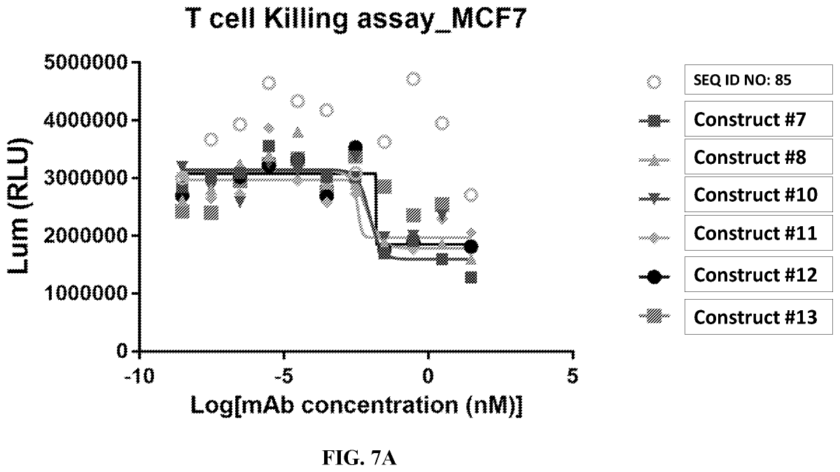

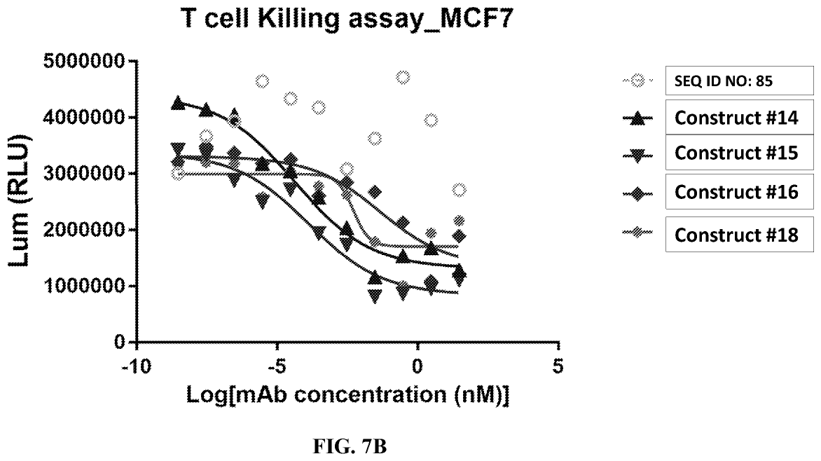

Results of T cell killing on MCF7 cancer cell line are shown in FIG. 7A and FIG. 7B. Table 27 summarizes the IC50s from the plots shown in FIG. 7A and FIG. 7B. The percent of dead cancer cells vs. log (concentration), applying a nonlinear regression (curve fit) of a log (inhibitor) vs. Response—variable slope (four parameters). IC50 values were calculated from the same analysis.

TABLE 27
IC50 (pM) of plots shown in FIG. 7A and FIG. 7B.

| Construct # | IC50 (pM) |
|---|---|
| SEQ ID NO: 85 | ND |
| 7 | 9.11 |
| 8 | 7.24 |
| 10 | 11.19 |
| 11 | 3.64 |
| 12 | 15.52 |
| 13 | ND |
| 14 | 0.04 |
| 15 | 0.13 |
| 16 | 38.43 |
| 18 | 3.3 |

ND = not determined.
Value underlined and italicized was estimated.

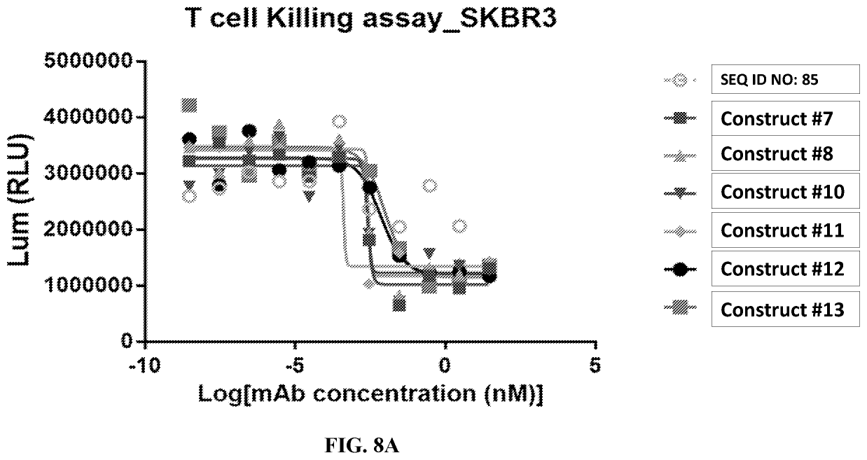

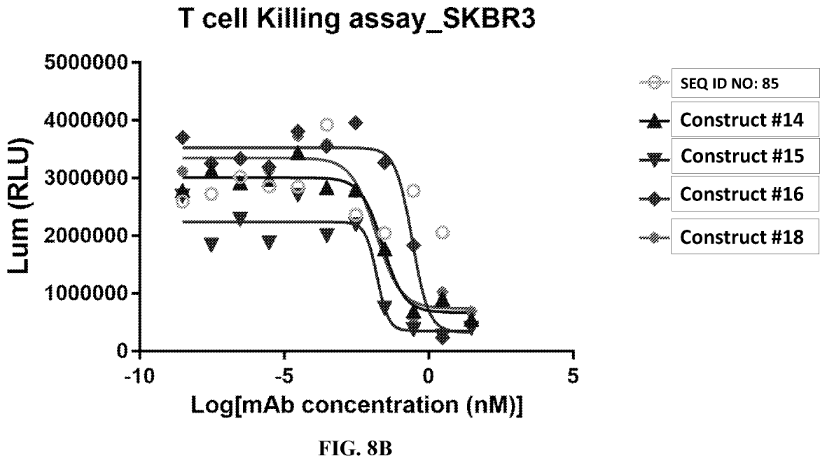

Results of T cell killing on SKBR3 cancer cell line are shown in FIG. 8A and FIG. 8B. Table 28 summarizes the IC50s from the plots shown in FIG. 8A and FIG. 8B. The percent of dead cancer cells vs. log (concentration), applying a nonlinear regression (curve fit) of a log (inhibitor) vs. Response—variable slope (four parameters). IC50 values were calculated from the same analysis.

TABLE 28
IC50 (pM) of plots shown in FIG. 8A and FIG. 8B.

| Construct # | IC50 (pM) |
|---|---|
| SEQ ID NO: 85 | ND |
| 7 | 2.7 |
| 8 | 2.82 |
| 10 | 2.82 |
| 11 | 0.41 |
| 12 | 7.34 |
| 13 | 10.0 |
| 14 | 25.66 |
| 15 | 16.32 |
| 16 | 271.3 |
| 18 | 15.02 |

ND = not determined.
Value underlined and italicized was estimated

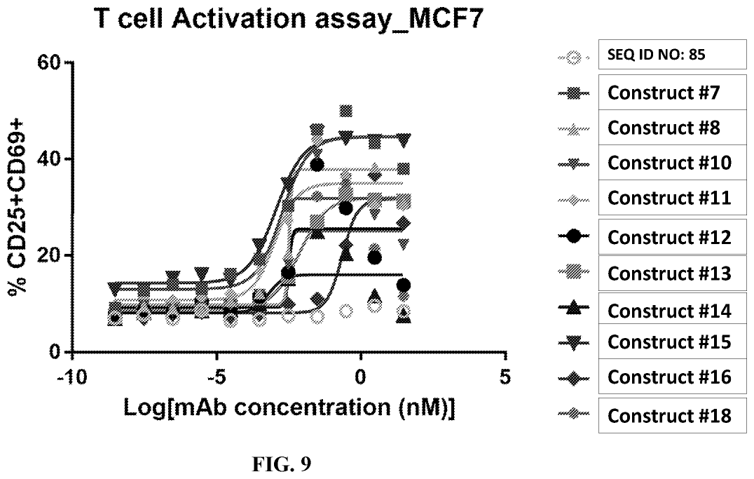

Results of the T cell activation assay in MCF7 cancer cell line is shown in FIG. 9. Table 29 summarizes the EC50s from the plot shown in FIG. 9. The plot illustrates percent of dead cancer cells vs. log (concentration) and plotted using GraphPad Prism 7, applying a nonlinear regression (curve fit) of a log (agonist) vs. Response—variable slop (four parameters). EC50 values were calculated from the same analysis.

TABLE 29
IC50 (pM) of plots shown in FIG. 9

| Construct # | EC50 (pM) |
|---|---|
| SEQ ID NO: 85 | ND |
| 7 | 1.86 |
| 8 | 3.2 |
| 10 | 3.11 |
| 11 | 1.03 |
| 12 | 3.09 |
| 13 | 7.13 |
| 14 | 0.62 |
| 15 | 1.12 |
| 16 | 221.8 |
| 18 | 3.18 |

ND = not determined.
Value underlined and italicized was estimated.

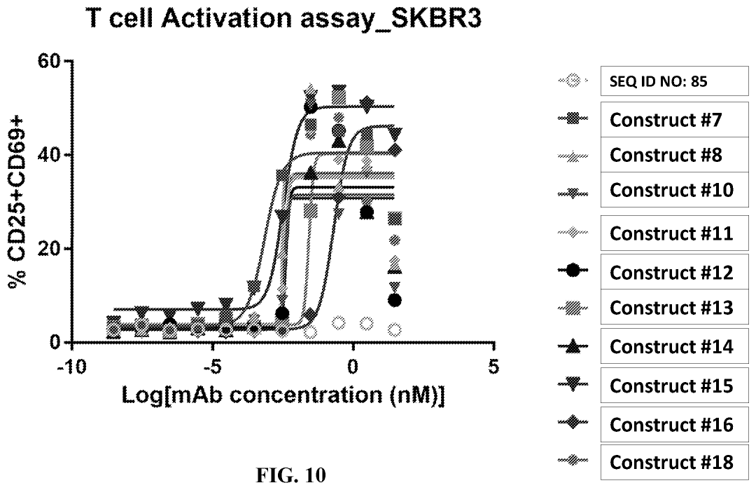

Results of the T cell activation assay in SKBR3 cancer cell line is shown in FIG. 10. Table 30 summarizes the EC50s from the plot shown in FIG. 10. The plot illustrates percent of dead cancer cells vs. log (concentration) and plotted using GraphPad Prism 7, applying a nonlinear regression (curve fit) of a log (agonist) vs. Response—variable slop (four parameters). EC50 values were calculated from the same analysis.

TABLE 30
IC50 (pM) of plots shown in FIG. 10 Construct numbers 14 and 15
have the best T cell killing activity on MCF7 cells and Construct
11 has the best T cell killing activity on SKBR3 cells.

| Construct # | EC50 (pM) |
|---|---|
| SEQ ID NO: 85 | ND |
| 7 | 0.71 |
| 8 | 3.16 |
| 10 | 3.88 |
| 11 | 3.64 |
| 12 | 3.25 |
| 13 | 29.02 |
| 14 | 3.67 |
| 15 | 3.22 |
| 16 | 202.7 |
| 18 | 4.01 |

ND = not determined.
Value underlined and italicized was estimated.

### Example 4

### SCP-CD3/HER2 Affinity Determination

The aim of this Example is to determine by SPR, using Biacore 8k+, the affinity and avidity of single chain polypeptide constructs to bio-huCD3, huHER2, and cyHER2 proteins in multi cycle kinetics (MCK). Constructs were injected for binding in-solution to the immobilized proteins. The setup for affinity determination used the Biacore 8K+ equipment, the chip used was a CFJB755 CM5 Series S sensor chip (Cytivia, Cat nr. 29104992) immobilized by amine coupling with biotinylated huCD3ε/δ (AcroBiosystems; Cat. nr. CDD-H82W0), huHER2-His (AcroBiosystems; Cat. nr. HE2-H5225-100ug) and cyHER2-His (AcroBiosystems; Cat. nr. HE2-C52Hb-100ug) at low density (1000-3000 RUs) on flow channels 1, 3 and 5 and at high density (6200-7000 RUs) on flow channels 2, 4 and 6. The single chain polypeptide construct samples were injected at 0.3, 0.6, 1.3, 2.5 and 5 nM in flow cells 1 and 2 for 2 minutes at 30 μL/min. The control monoclonal antibody was injected at 10 nM in flow cells 1 and 2 for 2 minutes at 30 μL/min. For flow channels 1-2, mouse anti-huCD3 (R&D; Cat. nr. MAB100-100) was used. For flow channels 3-6, human anti-HER2 (R&D; Cat. nr. MAB9589-100) was used. The off-rate measurement was 300 seconds. For regeneration, baseline levels were restored using a regeneration solution. The buffer used was 1×HBS-EP+ pH 7.4. The data was analyzed using the multi cycle kinetics predefined evaluation method of the Biacore Insight Evaluation Software. Kinetic parameters were calculated using the 1:1 binding fitting model.

Tables 31A and 31B summarize the huCD3 results. Tables 32A and 32B summarize the huHER2 results. Tables 33A and 33B summarize the cyHER2 results. Table 34 shows the KDs for CD3 binding are more than 100-fold higher, and in one case, over 10,000-fold higher, than the HER2 control (Construct #47) and the 3.8 nM KD reported for anti-CD3 2B2 (SEQ ID NO: 75 for VH; and SEQ ID NO: 76 for VL). In addition, the CD3 configuration of the constructs tested retain binding to cynomolgus monkey CD3. The branched construct with 4 VHHs (Construct #18) binds to huHER2 with a 100-fold lower KD. Both HER2 and EGFR constructs with 6, 7 and 8 residue linkers between the CD3 domains are active.

TABLE 31A

| | | | Construct | | Kinetics |
|---|---|---|---|---|---|
| Con- | Immobilized | Immobilized | Concentration | Rmax | Chi² |
| struct # | ligand | density | (nM) | (RU) | (RU²) |
| SEQ ID | bio-huCD3 | Low | 0.3-2.5 nM | 369 | 0.95 |
| NO: 85 | bio-huCD3 | High | 0.3-2.5 nM | 249 | 0.79 |
| 7 | bio-huCD3 | Low | 0.3-2.5 nM | 807 | 2.77 |
| | bio-huCD3 | High | 0.3-2.5 nM | 525 | 1.88 |
| 8 | bio-huCD3 | Low | 0.3-2.5 nM | 890 | 2.64 |
| | bio-huCD3 | High | 0.3-2.5 nM | 382 | 1.40 |
| 10 | bio-huCD3 | Low | 0.3-2.5 nM | 668 | 3.41 |
| | bio-huCD3 | High | 0.3-2.5 nM | 562 | 2.75 |
| 11 | bio-huCD3 | Low | 0.3-2.5 nM | 731 | 3.31 |
| | bio-huCD3 | High | 0.3-2.5 nM | 355 | 2.29 |
| 12 | bio-huCD3 | Low | 0.3-2.5 nM | 770 | 2.94 |
| | bio-huCD3 | High | 0.3-2.5 nM | 625 | 2.27 |
| 13 | bio-huCD3 | Low | 0.3-2.5 nM | 211 | 0.13 |
| | bio-huCD3 | High | 0.3-2.5 nM | 127 | 0.32 |
| 14 | bio-huCD3 | Low | 0.3-2.5 nM | 783 | 2.80 |
| | bio-huCD3 | High | 0.3-2.5 nM | 582 | 3.40 |
| 15 | bio-huCD3 | Low | 0.3-2.5 nM | 740 | 4.95 |
| | bio-huCD3 | High | 0.3-2.5 nM | 982 | 4.62 |
| 16 | bio-huCD3 | Low | 0.3-2.5 nM | N.D. | N.D. |
| | bio-huCD3 | High | 0.3-2.5 nM | N.D. | N.D. |
| 18 | bio-huCD3 | Low | 0.3-2.5 nM | 283 | 1.83 |
| | bio-huCD3 | High | 0.3-2.5 nM | 316 | 1.05 |

TABLE 31B

| Construct # | ka (1/Ms) | kd (1/s) | KD (M) | KD (nM) |
|---|---|---|---|---|
| SEQ ID | 4.1E+06 | 1.1E−04 | 2.7E−11 | 0.03 |
| NO: 85 | 1.4E+07 | 3.9E−06 | 2.8E−13 | 0.0003 |
| 7 | 1.7E+06 | 7.6E−05 | 4.5E−11 | 0.04 |
| | 4.2E+06 | 1.3E−05 | 3.0E−12 | 0.003 |
| 8 | 1.6E+06 | 1.3E−04 | 8.5E−11 | 0.08 |
| | 6.7E+06 | 1.0E−04 | 1.5E−11 | 0.02 |
| 10 | 3.5E+06 | 1.8E−04 | 5.0E−11 | 0.05 |
| | 4.3E+06 | 1.9E−05 | 4.5E−12 | 0.004 |
| 11 | 2.1E+06 | 7.3E−05 | 3.4E−11 | 0.03 |
| | 9.6E+06 | <1.0E−06 | <1.0E−13 | <0.0001 |
| 12 | 1.9E+06 | 1.1E−04 | 5.9E−11 | 0.06 |
| | 2.4E+06 | 1.7E−05 | 6.8E−12 | 0.01 |
| 13 | 8.2E+05 | 5.5E−05 | 6.7E−11 | 0.07 |
| | 1.4E+06 | 3.5E−05 | 2.5E−11 | 0.03 |
| 14 | 1.8E+06 | 1.1E−04 | 6.3E−11 | 0.06 |
| | 3.3E+06 | 4.5E−05 | 1.3E−11 | 0.01 |
| 15 | 1.9E+06 | 6.6E−05 | 3.5E−11 | 0.03 |
| | 8.4E+05 | 1.3E−06 | 1.6E−12 | 0.002 |
| 16 | N.D. | N.D. | N.D. | N.D. |
| | N.D. | N.D. | N.D. | N.D. |
| 18 | 2.3E+07 | 7.0E−05 | 3.0E−12 | 0.0030 |
| | 1.1E+07 | 3.1E−06 | 2.9E−13 | 0.0003 |

TABLE 32A

| | | | Construct | | Kinetics |
|---|---|---|---|---|---|
| Con- | Immobilized | Immobilized | Concentration | Rmax | Chi² |
| struct # | ligand | density | (nM) | (RU) | (RU²) |
| SEQ ID | huHER2 | Low | 0.3-2.5 nM | N.D. | N.D. |
| NO: 85 | huHER2 | High | 0.3-2.5 nM | N.D. | N.D. |
| 7 | huHER2 | Low | 0.3-2.5 nM | 655 | 3.18 |
| | huHER2 | High | 0.3-2.5 nM | 1047 | 1.97 |
| 8 | huHER2 | Low | 0.3-2.5 nM | 1437 | 1.46 |
| | huHER2 | High | 0.3-2.5 nM | 723 | 1.55 |
| 10 | huHER2 | Low | 0.3-2.5 nM | 1030 | 3.47 |
| | huHER2 | High | 0.3-2.5 nM | 746 | 2.49 |
| 11 | huHER2 | Low | 0.3-2.5 nM | 655 | 4.60 |
| | huHER2 | High | 0.3-2.5 nM | 757 | 2.73 |
| 12 | huHER2 | Low | 0.3-2.5 nM | 2059 | 3.94 |
| | huHER2 | High | 0.3-2.5 nM | 897 | 2.00 |
| 13 | huHER2 | Low | 0.3-2.5 nM | 132 | 0.48 |
| | huHER2 | High | 0.3-2.5 nM | 382 | 0.85 |
| 14 | huHER2 | Low | 0.3-2.5 nM | 1426 | 5.38 |
| | huHER2 | High | 0.3-2.5 nM | 696 | 4.54 |
| 15 | huHER2 | Low | 0.3-2.5 nM | 8965 | 9.29 |
| | huHER2 | High | 0.3-2.5 nM | 797 | 7.95 |
| 16 | huHER2 | Low | 0.3-2.5 nM | 98 | 0.17 |
| | huHER2 | High | 0.3-2.5 nM | 196 | 1.04 |
| 18 | huHER2 | Low | 0.3-2.5 nM | 588 | 1.72 |
| | huHER2 | High | 0.3-2.5 nM | 427 | 1.22 |

TABLE 32B

| | ka | kd | KD | KD |
|---|---|---|---|---|
| Construct # | (1/Ms) | (1/s) | (M) | (nM) |
| SEQ ID | N.D. | N.D. | N.D. | N.D. |
| NO: 85 | N.D. | N.D. | N.D. | N.D. |
| 7 | 9.2E+06 | 6.9E−04 | 7.5E−11 | 0.07 |
| | 1.5E+06 | 8.9E−05 | 6.0E−11 | 0.06 |
| 8 | 1.0E+06 | 3.2E−04 | 3.2E−10 | 0.32 |
| | 3.7E+06 | 2.9E−04 | 7.7E−11 | 0.08 |
| 10 | 5.1E+06 | 2.0E−04 | 3.9E−11 | 0.04 |
| | 4.3E+06 | 1.1E−04 | 2.6E−11 | 0.03 |
| 11 | 8.3E+06 | 1.7E−04 | 2.0E−11 | 0.02 |
| | 3.2E+06 | 6.4E−05 | 2.0E−11 | 0.02 |
| 12 | 8.2E+06 | 1.0E−03 | 1.3E−10 | 0.13 |
| | 1.4E+06 | 3.2E−05 | 2.2E−11 | 0.02 |
| 13 | 1.3E+07 | 4.3E−05 | 3.3E−12 | 0.003 |
| | 9.3E+05 | 7.4E−05 | 8.0E−11 | 0.08 |
| 14 | 9.8E+06 | 9.0E−04 | 9.2E−11 | 0.09 |
| | 4.1E+06 | 3.5E−05 | 8.7E−12 | 0.01 |
| 15 | 7.5E+06 | 2.6E−03 | 3.5E−10 | 0.35 |
| | 3.0E+06 | 4.8E−05 | 1.6E−11 | 0.02 |
| 16 | 1.7E+06 | 2.1E−05 | 1.3E−11 | 0.01 |
| | 1.5E+06 | 1.7E−05 | 1.2E−11 | 0.01 |
| 18 | 5.1E+06 | 2.3E−06 | 4.5E−13 | 0.0005 |
| | 8.7E+06 | <1.0E−06 | <1.1E−13 | <0.00011 |

TABLE 33A

| | | | Construct | | Kinetics |
|---|---|---|---|---|---|
| Con- | Immobilized | Immobilized | Concentration | Rmax | Chi² |
| struct # | ligand | density | (nM) | (RU) | (RU²) |
| SEQ ID | cyHER2 | Low | 0.3-2.5 nM | N.D. | N.D. |
| NO: 85 | cyHER2 | High | 0.3-2.5 nM | N.D. | N.D. |
| 7 | cyHER2 | Low | 0.3-2.5 nM | N.A. | N.A. |
| | cyHER2 | High | 0.3-2.5 nM | N.A. | N.A. |
| 8 | cyHER2 | Low | 0.3-2.5 nM | N.A. | N.A. |
| | cyHER2 | High | 0.3-2.5 nM | N.A. | N.A. |
| 10 | cyHER2 | Low | 0.3-2.5 nM | N.A. | N.A. |
| | cyHER2 | High | 0.3-2.5 nM | N.A. | N.A. |
| 11 | cyHER2 | Low | 0.3-2.5 nM | N.A. | N.A. |
| | cyHER2 | Low | 0.3-2.5 nM | N.A. | N.A. |
| 12 | cyHER2 | High | 0.3-2.5 nM | N.A. | N.A. |
| | cyHER2 | Low | 0.3-2.5 nM | N.A. | N.A. |
| 13 | cyHER2 | High | 0.3-2.5 nM | 478 | 0.40 |
| | cyHER2 | Low | 0.3-2.5 nM | 310 | 1.25 |
| 14 | cyHER2 | High | 0.3-2.5 nM | N.A. | N.A. |
| | cyHER2 | Low | 0.3-2.5 nM | N.A. | N.A. |
| 15 | cyHER2 | High | 0.3-2.5 nM | N.A. | N.A. |
| | cyHER2 | Low | 0.3-2.5 nM | N.A. | N.A. |
| 16 | cyHER2 | High | 0.3-2.5 nM | 97 | 0.42 |
| | cyHER2 | Low | 0.3-2.5 nM | 156 | 1.18 |
| 18 | cyHER2 | High | 0.3-2.5 nM | 304 | 2.10 |
| | cyHER2 | Low | 0.3-2.5 nM | 455 | 2.40 |

TABLE 33B

| | | | Construct | | Kinetics |
|---|---|---|---|---|---|
| Con- | Immobilized | Immobilized | Concentration | Rmax | Chi² |
| struct # | ligand | density | (nM) | (RU) | (RU²) |
| SEQ ID | cyHER2 | Low | 0.3-2.5 nM | N.D. | N.D. |
| NO: 85 | cyHER2 | High | 0.3-2.5 nM | N.D. | N.D. |
| 7 | cyHER2 | Low | 0.3-2.5 nM | N.A. | N.A. |
| | cyHER2 | High | 0.3-2.5 nM | N.A. | N.A. |
| 8 | cyHER2 | Low | 0.3-2.5 nM | N.A. | N.A. |
| | cyHER2 | High | 0.3-2.5 nM | N.A. | N.A. |
| 10 | cyHER2 | Low | 0.3-2.5 nM | N.A. | N.A. |
| | cyHER2 | High | 0.3-2.5 nM | N.A. | N.A. |
| 11 | cyHER2 | Low | 0.3-2.5 nM | N.A. | N.A. |
| | cyHER2 | Low | 0.3-2.5 nM | N.A. | N.A. |
| 12 | cyHER2 | High | 0.3-2.5 nM | N.A. | N.A. |
| | cyHER2 | Low | 0.3-2.5 nM | N.A. | N.A. |
| 13 | cyHER2 | High | 0.3-2.5 nM | 478 | 0.40 |
| | cyHER2 | Low | 0.3-2.5 nM | 310 | 1.25 |
| 14 | cyHER2 | High | 0.3-2.5 nM | N.A. | N.A. |
| | cyHER2 | Low | 0.3-2.5 nM | N.A. | N.A. |
| 15 | cyHER2 | High | 0.3-2.5 nM | N.A. | N.A. |
| | cyHER2 | Low | 0.3-2.5 nM | N.A. | N.A. |
| 16 | cyHER2 | High | 0.3-2.5 nM | 97 | 0.42 |
| | cyHER2 | Low | 0.3-2.5 nM | 156 | 1.18 |
| 18 | cyHER2 | High | 0.3-2.5 nM | 304 | 2.10 |
| | cyHER2 | Low | 0.3-2.5 nM | 455 | 2.40 |

TABLE 34

| | | | Kinetics | | | Estimated | | |
|---|---|---|---|---|---|---|---|---|
| Construct | Density | Concentration | Chi² | ka | kd | Rmax | KD | KD |
| ID | Immobilization | (nM) | (RU²) | (1/Ms) | (1/s) | (RU) | (M) | (nM) |
| 19 | Low | 0.3-5 | 2.2 | 2.20E+06 | 7.20E−03 | 2630.9 | 3.30E−09 | 3.3 |
| 19 | High | 0.3-5 | 1 | 1.90E+06 | 1.10E−03 | 1932.7 | 5.80E−10 | 0.6 |
| 21 | Low | 0.3-5 | 0.6 | 3.20E+06 | 1.60E−06 | 4412.2 | 4.90E−13 | 0.0005 |
| 21 | High | 0.3-5 | 2.1 | 1.60E+06 | 1.70E−03 | 3113.9 | 1.10E−09 | 1.1 |
| 22 | Low | 0.3-5 | 2.1 | 2.40E+06 | 6.70E−03 | 3728.6 | 2.80E−09 | 2.8 |
| 22 | High | 0.3-5 | 3.4 | 1.30E+06 | 1.50E−03 | 1920.9 | 1.10E−09 | 1.1 |
| 29 | Low | 0.3-5 | 0.5 | 2.50E+06 | 3.20E−06 | 3132.4 | 1.30E−12 | 0.001 |
| 29 | High | 0.3-5 | 1.3 | 6.60E+06 | 7.50E−03 | 2636.5 | 1.10E−09 | 1.1 |
| 30 | Low | 0.3-5 | 0.6 | 2.30E+06 | 5.10E−03 | 1917.2 | 2.30E−09 | 2.3 |
| 30 | High | 0.3-5 | 1.4 | 1.90E+06 | 1.50E−03 | 1988.7 | 8.10E−10 | 0.8 |
| 31 | Low | 0.3-5 | 0.4 | 3.80E+06 | 6.70E−03 | 1972.5 | 1.80E−09 | 1.8 |
| 31 | High | 0.3-5 | 3.8 | 1.50E+06 | 3.60E−03 | 1302.6 | 2.40E−09 | 2.4 |
| 32 | Low | 0.3-5 | 0.8 | 1.50E+06 | 2.80E−03 | 1303.3 | 1.90E−09 | 1.9 |
| 32 | High | 0.3-5 | 0.7 | 1.80E+06 | 1.70E−03 | 1587 | 9.20E−10 | 0.9 |
| 39 | Low | 0.3-5 | 0.5 | 2.70E+06 | 3.70E−03 | 1446.5 | 1.30E−09 | 1.3 |
| 39 | High | 0.3-5 | 2 | 1.50E+06 | 2.00E−03 | 2456.6 | 1.30E−09 | 1.3 |
| 40 | Low | 0.3-5 | 0.3 | 2.80E+06 | 1.40E−03 | 1003.6 | 5.00E−10 | 0.5 |
| 40 | High | 0.3-5 | 3.5 | 2.50E+06 | 3.10E−03 | 799.4 | 1.20E−09 | 1.2 |
| 41 | 20 μg/mL bio- | 0.3-5 | 0 | N.D | N.D | <10 | N.D | N.D |
| | huCD3εδ | | | | | | | |
| 41 | 40 μg/mL bio- | 0.3-5 | 0.5 | N.D | N.D | <10 | N.D | N.D |
| | huCD3εδ | | | | | | | |
| 42 | 20 μg/mL bio- | 0.3-5 | 0.6 | 6.60E+06 | 1.00E−02 | 1006.5 | 1.50E−09 | 1.5 |
| | huCD3εδ | | | | | | | |
| 42 | 40 μg/mL bio- | 0.3-5 | 2 | 5.10E+06 | 6.80E−03 | 1512.5 | 1.30E−09 | 1.3 |
| | huCD3εδ | | | | | | | |
| 47 | 20 μg/mL bio- | 0.3-5 | 0.9 | 2.30E+06 | 2.30E−03 | 3738.4 | 1.00E−09 | 1 |
| | huCD3εδ | | | | | | | |
| 47 | 40 μg/mL bio- | 0.3-5 | 1.2 | 1.90E+06 | 3.30E−03 | 1298.9 | 1.70E−09 | 1.7 |
| | huCD3εδ | | | | | | | |

### Example 5

### Efficacy of Single Chain Polypeptides in a Breast Cancer PDX Model in NSG Mice

NOG mice are inoculated subcutaneously (SC) with 2×10⁷ BT-474 tumor cells, engrafted with 1×107 huPBMCs on Day 8 and treated 2 days later at metabolic tumor volume (MTV) with the antibody at two equimolar doses (e.g., 5 nmol/kg and 10 nmol/kg daily for 3 weeks). Mice are weighed once weekly, and subsequently are sacrificed on day 38 to permit collection of organs for analysis by flow cytometry.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A single chain polypeptide comprising variable domains connected by linkers that fold into a conformation such that the variable domains form two effector cell antigen interacting domains (E) wherein each effector cell antigen interacting domain comprises two variable domains of a single chain variable fragment (EV and EV), and at least one of the effector cell antigen interacting domains is linked to one or more target interacting domains.

2. The single chain polypeptide of claim 1, wherein the target interacting domain binds to a tumor antigen, a ligand, a protein, a lipid, nucleic acid, or a polysaccharide.

3. The single chain polypeptide of claim 2, wherein the tumor antigen is on a cell in a tumor microenvironment.

4. The single chain polypeptide of any one of claims 1-3, wherein the target interacting domain comprises an antibody, an antibody fragment, a ligand, or a cofactor.

5. The single chain polypeptide of claim 4, wherein the antibody or antibody fragment of the targeting interacting domain comprises a Fab, a Fab′, a F(ab′)₂, a Fv fragment, a single-chain Fv (scFv), a tandem single-chain Fv ((scFv)₂, a dual affinity retargeting antibody, a diabody, a VHH single domain antibody, a TriTac, an Adnectin, an Anticalin, an Avimer, a Fynomer, a Kunitz domain, a knottin, an Affibody, or a DARPin.

6. The single chain polypeptide of any one of claims 1-5, wherein the one or more target interacting domains comprises one or more target cell antigen interacting domains that bind to a target cell antigen.

7. The single chain polypeptide of claim 6, wherein the target cell antigen interacting domain binds to more than one target cell antigen.

8. The single chain polypeptide of claim 6 or 7, wherein the target cell antigen interacting domain is a bispecific or multispecific antibody or antibody fragment.

9. The single chain polypeptide of any one of claims 6-8, wherein the target cell antigen interacting domain comprises a VHH single domain antibody (TVHH).

10. The single chain polypeptide of any one of claims 1-9, wherein the single chain polypeptide does not contain a fragment crystallizable (Fc) domain.

11. The single chain polypeptide of any one of claims 1-10, wherein the two variable domains of the single chain variable fragment (EV and EV) comprise a variable heavy chain of a single chain variable fragment (EVH) and a variable light chain of single chain variable fragment (EVL).

12. The single chain polypeptide of claim 11, wherein the EVL of the at least one of the effector cell antigen interacting domains that is linked to the one or more target cell antigen interacting domains is linked to at least one TVHH, and the EVH of the one of the effector cell antigen interacting domains that is linked to the one or more target cell antigen interacting domains is linked to at least one TVHH.

13. The single chain polypeptide of claim 12, wherein the at least one TVHH that is linked to the EVL binds to the same target antigen as the at least one TVHH that is linked to the EVH.

14. The single chain polypeptide of claim 13, wherein the at least one TVHH that is linked to the EVL binds to the same target antigen and has the same complementarity determining region (CDR) sequences as the at least one TVHH that is linked to the EVH.

15. The single chain polypeptide of claim 13, wherein the at least one TVHH that is linked to the EVL binds to the same target antigen and have no complementarity determining region (CDR) sequences in common as the at least one TVHH that is linked to the EVH.

16. The single chain polypeptide of claim 12, wherein the at least one TVHH that is linked to the EVL binds to a different target antigen as compared to the at least one TVHH that is linked to the EVH.

17. The single chain polypeptide of any one of claims 11-16, wherein the EVL and the EVH are linked to the same number of TVHHs.

18. The single chain polypeptide of any one of claims 11-16, wherein the EVL is linked to a different number of TVHHs as compared to the number of TVHHs that are linked to the EVH.

19. The single chain polypeptide of claim 17, wherein the EVL of the at least one of the effector cell antigen interacting domains that is linked to the one or more target cell antigen interacting domains is linked to one TVHH, and the EVH of the at least one of the effector cell antigen interacting domains that is linked to the one or more target cell antigen interacting domains is linked to one TVHH.

20. The single chain polypeptide of claim 18, wherein the EVL of the at least one of the effector cell antigen interacting domains that is linked to the one or more target cell antigen interacting domains is linked to one TVHH, and the EVH of the at least one of the effector cell antigen interacting domains that is linked to the one or more target cell antigen interacting domains is linked to two TVHH.

21. The single chain polypeptide of claim 18, wherein the EVL of the at least one of the effector cell antigen interacting domains that is linked to the at least one or more target cell antigen interacting domains is linked to one TVHH, and the EVH of the at least one of the effector cell antigen interacting domains that is linked to the one or more target cell antigen interacting domains is linked to three TVHH.

22. The single chain polypeptide of claim 18, wherein the EVH of the at least one of the effector cell antigen interacting domains that is linked to the one or more target cell antigen interacting domains is linked to one TVHH, and the EVL of the at least one of the effector cell antigen interacting domains that is linked to the one or more target cell antigen interacting domains is linked to two TVHH.

23. The single chain polypeptide of claim 18, wherein the EVH of the at least one of the effector cell antigen interacting domains that is linked to the one or more target cell antigen interacting domains is linked to one TVHH, and the EVL of the at least one of the effector cell antigen interacting domains that is linked to the one or more target cell antigen interacting domains is linked to three TVHH.

24. The single chain polypeptide of claim 17, wherein the EVH of the at least one of the effector cell antigen interacting domains that is linked to the one or more target cell antigen interacting domains is linked to two TVHH, and the EVL of the at least one of the effector cell antigen interacting domains that is linked to the one or more target cell antigen interacting domains is linked to two TVHH.

25. The single chain polypeptide of claim 17, wherein the EVH of the at least one of the effector cell antigen interacting domains that is linked to the one or more target cell antigen interacting domains is linked to three TVHH, and the EVL of the at least one of the effector cell antigen interacting domains that is linked to the one or more target cell antigen interacting domains is linked to three TVHH.

26. The single chain polypeptide of claim 17, wherein the EVH of the at least one of the effector cell antigen interacting domains that is linked to the one or more target cell antigen interacting domains is linked to four TVHH, and the EVL of the at least one of the effector cell antigen interacting domains that is linked to the one or more target cell antigen interacting domains is linked to four TVHH.

27. The single chain polypeptide of claim 17, wherein the EVH of the at least one of the effector cell antigen interacting domains that is linked to the one or more target cell antigen interacting domains is linked to five TVHH, and the EVL of the at least one of the effector cell antigen interacting domains that is linked to the one or more target cell antigen interacting domains is linked to five TVHH.

28. The single chain polypeptide of claim 17, wherein the EVH of the at least one of the effector cell antigen interacting domains that is linked to the one or more target cell antigen interacting domains is linked to six TVHH, and the EVL of the at least one of the effector cell antigen interacting domains that is linked to the one or more target cell antigen interacting domains is linked to six TVHH.

29. The single chain polypeptide of claim 17, wherein the EVH of the at least one of the effector cell antigen interacting domains that is linked to the one or more target cell antigen interacting domains is linked to seven TVHH, and the EVL of the at least one of the effector cell antigen interacting domains that is linked to the one or more target cell antigen interacting domains is linked to seven TVHH.

30. The single chain polypeptide of claim 17, wherein the EVH of the at least one of the effector cell antigen interacting domains that is linked to the one or more target cell antigen interacting domains is linked to eight TVHH, and the EVL of the at least one of the effector cell antigen interacting domains that is linked to the one or more target cell antigen interacting domains is linked to eight TVHH.

31. The single chain polypeptide of claim 11, wherein the at least one of the effector cell antigen interacting domains that is linked to the one or more target cell antigen interacting domains is linked to one target cell antigen interacting domain.

32. The single chain polypeptide of claim 31, wherein the one or more target cell antigen interacting domain is linked C-terminal to the at least one of the effector cell antigen interacting domains.

33. The single chain polypeptide of claim 31, wherein the one or more target cell antigen interacting domains is linked N-terminal to the at least one of the effector cell antigen interacting domains.

34. The single chain polypeptide of claim 11, wherein the at least one of the effector cell antigen interacting domains that is linked to the one or more target cell antigen interacting domains is linked to more than one target cell antigen interacting domain.

35. The single chain polypeptide of claim 34, wherein all of the more than one target cell antigen interacting domains bind to the same target antigen.

36. The single chain polypeptide of claim 35, wherein all of the more than one target cell antigen interacting domains bind to the same target antigen and all have the same CDR sequences.

37. The single chain polypeptide of claim 35, wherein all of the more than one target cell antigen interacting domains bind to the same target antigen and at least two of the more than one target cell antigen interacting domains have no CDR sequences in common.

38. The single chain polypeptide of claim 34, wherein at least two of the more than one target cell antigen interacting domains bind to different target antigens.

39. The single chain polypeptide of claim 11, wherein the at least one of the effector cell antigen interacting domains that is linked to the one or more target cell antigen interacting domains is linked to two target cell antigen interacting domains.

40. The single chain polypeptide of claim 39, wherein the two target cell antigen interacting domains are linked C-terminal to the one of the effector cell antigen interacting domains.

41. The single chain polypeptide of claim 39, wherein the two target cell antigen interacting domains are linked N-terminal to the one of the effector cell antigen interacting domains.

42. The single chain polypeptide of claim 11, wherein the at least one target one of the effector cell antigen interacting domains that is linked to the one or more target cell antigen interacting domains is linked to three target cell antigen interacting domains.

43. The single chain polypeptide of claim 42, wherein the three target cell antigen interacting domains are linked C-terminal to the one of the effector cell antigen interacting domains.

44. The single chain polypeptide of claim 42, wherein the three target cell antigen interacting domains are linked N-terminal to the one of the effector cell antigen interacting domains.

45. The single chain polypeptide of claim 11, wherein the at least one target one of the effector cell antigen interacting domains that is linked to the one or more target cell antigen interacting domains is linked to four target cell antigen interacting domains.

46. The single chain polypeptide of claim 45, wherein the four target cell antigen interacting domains are linked C-terminal to the one of the effector cell antigen interacting domains.

47. The single chain polypeptide of claim 45, wherein the four target cell antigen interacting domains are linked N-terminal to the one of the effector cell antigen interacting domains.

48. The single chain polypeptide of any one of claims 9-47, wherein the variable domains and the one or more target cell antigen interacting domains are ordered according to the following N-terminal to C-terminal arrangements in which (-) is the linker:
TVHH-EV-EV-EV-EV;
EV-EV-EV-EV-TVHH;
TVHH-TVHH-EV-EV-EV-EV;
EV-EV-EV-EV-TVHH-TVHH;
TVHH-TVHH-TVHH-EV-EV-EV-EV;
EV-EV-EV-EV-TVHH-TVHH-TVHH;
TVHH-TVHH-TVHH-TVHH-EV-EV-EV-EV;
EV-EV-EV-EV-TVHH-TVHH-TVHH-TVHH;
TVHH-EV-EV-EV-EV-TVHH;
TVHH-EV-EV-EV-EV-TVHH-TVHH;
TVHH-TVHH-EV-EV-EV-EV-TVHH;
TVHH-EV-EV-EV-EV-TVHH-TVHH-TVHH;
TVHH-TVHH-TVHH-EV-EV-EV-EV-TVHH;
TVHH-TVHH-EV-EV-EV-EV-TVHH-TVHH;
TVHH-TVHH-TVHH-EV-EV-EV-EV-TVHH-TVHH-TVHH;
TVHH-TVHH-TVHH-TVHH-EV-EV-EV-EV-TVHH-
TVHH-TVHH-TVHH;
TVHH-TVHH-TVHH-TVHH-TVHH-EV-EV-EV-EV-
TVHH-TVHH-TVHH-TVHH-TVHH;
TVHH-TVHH-TVHH-TVHH-TVHH-TVHH-EV-EV-
EV-EV-TVHH-TVHH-TVHH-TVHH-TVHH-TVHH;
TVHH-TVHH-TVHH-TVHH-TVHH-TVHH-TVHH-EV-
EV-EV-EV-TVHH-TVHH-TVHH-TVHH-TVHH-TVHH-TVHH;
or
TVHH-TVHH-TVHH-TVHH-TVHH-TVHH-TVHH-
TVHH-EV-EV-EV-EV-TVHH-TVHH-TVHH-
TVHH-TVHH-TVHH-TVHH-TVHH.

49. The single chain polypeptide of claim 48, wherein the variable domains and the one or more target cell antigen interacting domains are ordered according to the following N-terminal to C-terminal arrangements in which (-) is the linker:
TVHH-TVHH-EV-EV-EV-EV-TVHH-TVHH;
or
TVHH-EV-EV-EV-EV-TVHH.

50. The single chain polypeptide of claim 48, wherein the variable domains and the one or more target cell antigen interacting domains are ordered according to the following N-terminal to C-terminal arrangements in which (-) is the linker:
TVHH-TVHH-EV-EV-EV-EV-TVHH-TVHH.

51. The single chain polypeptide of claim 48, wherein the variable domains and the one or more target cell antigen interacting domains are ordered according to the following N-terminal to C-terminal arrangements in which (-) is the linker:
TVHH-EV-EV-EV-EV-TVHH.

52. The single chain polypeptide of claim 48, wherein the variable domains and the one or more target cell antigen interacting domains are ordered according to the following N-terminal to C-terminal arrangements in which (-) is the linker: TVHH-TVHH-TVHH-TVHH-TVHH-TVHH-TVHH-TVHH-EV-EV-EV-EV-TVHH-TVHH-TVHH-TVHH-TVHH-TVHH-TVHH-TVHH.

53. The single chain polypeptide of any one of claims 11-52, wherein the variable domains and the one or more target cell antigen interacting domains are ordered according to the following N-terminal to C-terminal arrangements in which (-) is the linker:
TVHH-EVL-EVH-EVL-EVH;
TVHH-EVH-EVL-EVH-EVL;
EVL-EVH-EVL-EVH-TVHH;
EVH-EVL-EVH-EVL-TVHH;
TVHH-TVHH-EVL-EVH-EVL-EVH;
TVHH-TVHH-EVH-EVL-EVH-EVL;
EVL-EVH-EVL-EVH-TVHH-TVHH;
EVH-EVL-EVH-EVL-TVHH-TVHH;
TVHH-TVHH-TVHH-EVL-EVH-EVL-EVH;
TVHH-TVHH-TVHH-EVH-EVL-EVH-EVL;
EVL-EVH-EVL-EVH-TVHH-TVHH-TVHH;
EVH-EVL-EVH-EVL-TVHH-TVHH-TVHH;
TVHH-TVHH-TVHH-TVHH-EVL-EVH-EVL-EVH;
TVHH-TVHH-TVHH-TVHH-EVH-EVL-EVH-EVL;
EVL-EVH-EVL-EVH-TVHH-TVHH-TVHH-TVHH;
EVH-EVL-EVH-EVL-TVHH-TVHH-TVHH-TVHH;
TVHH-EVL-EVH-EVL-EVH-TVHH;
TVHH-EVH-EVL-EVH-EVL-TVHH;
TVHH-EVL-EVH-EVL-EVH-TVHH-TVHH;
TVHH-EVH-EVL-EVH-EVL-TVHH-TVHH;
TVHH-TVHH-EVL-EVH-EVL-EVH-TVHH;
TVHH-TVHH-EVH-EVL-EVH-EVL-TVHH;
TVHH-EVL-EVH-EVL-EVH-TVHH-TVHH-TVHH;
TVHH-EVH-EVL-EVH-EVL-TVHH-TVHH-TVHH;
TVHH-TVHH-TVHH-EVL-EVH-EVL-EVH-TVHH;
TVHH-TVHH-TVHH-EVH-EVL-EVH-EVL-TVHH;
TVHH-TVHH-EVL-EVH-EVL-EVH-TVHH-TVHH;
or
TVHH-TVHH-EVH-EVL-EVH-EVL-TVHH-TVHH.

54. The single chain polypeptide of claim 53, wherein the variable domains and the one or more target cell antigen interacting domains are ordered according to the following N-terminal to C-terminal arrangements in which (-) is the linker:
TVHH-TVHH-EVL-EVH-EVL-EVH-TVHH-TVHH;
TVHH-TVHH-EVH-EVL-EVH-EVL-TVHH-TVHH;
TVHH-EVL-EVH-EVL-EVH-TVHH;
or
TVHH-EVH-EVL-EVH-EVL-TVHH.

55. The single chain polypeptide of claim 53, wherein the variable domains and the one or more target cell antigen interacting domains are ordered according to the following N-terminal to C-terminal arrangements in which (-) is the linker:
TVHH-TVHH-EVL-EVH-EVL-EVH-TVHH-TVHH;
or
TVHH-TVHH-EVH-EVL-EVH-EVL-TVHH-TVHH.

56. The single chain polypeptide of claim 53, wherein the variable domains and the one or more target cell antigen interacting domains are ordered according to the following N-terminal to C-terminal arrangements in which (-) is the linker:
TVHH-EVL-EVH-EVL-EVH-TVHH;
or
TVHH-EVH-EVL-EVH-EVL-TVHH.

57. The single chain polypeptide of claim 53, wherein the variable domains and the one or more target cell antigen interacting domains are ordered according to the following N-terminal to C-terminal arrangements in which (-) is the linker:
TVHH-TVHH-EVL-EVH-EVL-EVH-TVHH-TVHH.

58. The single chain polypeptide of claim 53, wherein the variable domains and the one or more target cell antigen interacting domains are ordered according to the following N-terminal to C-terminal arrangements in which (-) is the linker:
TVHH-TVHH-EVH-EVL-EVH-EVL-TVHH-TVHH.

59. The single chain polypeptide of claim 53, wherein the variable domains and the one or more target cell antigen interacting domains are ordered according to the following N-terminal to C-terminal arrangements in which (-) is the linker:
TVHH-EVL-EVH-EVL-EVH-TVHH.

60. The single chain polypeptide of claim 53, wherein the variable domains and the one or more target cell antigen interacting domains are ordered according to the following N-terminal to C-terminal arrangements in which (-) is the linker:
TVHH-EVH-EVL-EVH-EVL-TVHH.

61. The single chain polypeptide of any one of claims 1-60, wherein the linkers that connect the two effector cell antigen interacting domains (E) to each other comprise internal linkers.

62. The single chain polypeptide of any one of claims 11-61, wherein the effector cell antigen interacting domain that is not connected directly to the one or more target cell antigen interacting domains comprises a terminal linker that connects EVL and EVH.

63. The single chain polypeptide of claim 61 or 62, wherein each of the internal linkers is 6-8 amino acids in length.

64. The single chain polypeptide of claim 62 or 63, wherein the terminal linker is 15 or 16 amino acids in length.

65. The single chain polypeptide of any one of claims 1-64, wherein each of the effector cell antigen interacting domains bind to an effector cell antigen.

66. The single chain polypeptide of claim 65, wherein the effector cell antigen comprises CD3, CD16a, or Death Receptor 5 (DR5).

67. The single chain polypeptide of claim 66, wherein the CD3 is CD3 delta, CD3 gamma, or CD3 epsilon.

68. The single chain polypeptide of any one of claims 65-67, wherein the effector cell antigen is on an effector cell.

69. The single chain polypeptide of claim 68, wherein the effector cell comprises a T cell, NK cell, or a macrophage.

70. The single chain polypeptide of any one of claims 6-69, wherein the target cell antigen interacting domains that interact with a target cell (T) that binds to the target cell antigen.

71. The single chain polypeptide of claim 70, wherein the target cell antigen is clustered in a lipid raft when on a cell associated with a disease state and not clustered in a lipid raft when on a cell that is not associated with a disease state.

72. The single chain polypeptide of claim 70, wherein the target cell antigen is clustered when on a cell associated with a disease state and not clustered when on a cell that is not associated with a disease state.

73. The single chain polypeptide of claim 70, wherein the target cell antigens are 100 nm or less when on a cell associated with a disease state and not within 100 nm when on a cell that is not associated with a disease state.

74. The single chain polypeptide of claim 70, wherein the target cell antigens are 75 nm or less when on a cell associated with a disease state and not within 75 nm when on a cell that is not associated with a disease state.

75. The single chain polypeptide of claim 70, wherein the target cell antigens are 50 nm or less when on a cell associated with a disease state and not within 50 nm when on a cell that is not associated with a disease state.

76. The single chain polypeptide of claim 70, wherein the target cell antigens are 25 nm or less when on a cell associated with a disease state and not within 25 nm when on a cell that is not associated with a disease state.

77. The single chain polypeptide of claim 70, wherein the target cell antigens are 10 nm or less when on a cell associated with a disease state and not within 10 nm when on a cell that is not associated with a disease state.

78. The single chain polypeptide of claim 70, wherein the target cell antigens are 5 nm or less when on a cell associated with a disease state and not within 5 nm when on a cell that is not associated with a disease state.

79. The single chain polypeptide of claim 70, wherein the target cell antigens are 2 nm or less when on a cell associated with a disease state and not within 2 nm when on a cell that is not associated with a disease state.

80. The single chain polypeptide of claim 70, wherein the target cell antigens are in close proximity when on a cell associated with a disease state and not within close proximity when on a cell that is not associated with a disease state.

81. The single chain polypeptide of any one of claims 70-80, wherein the target cell antigen is a dimer, trimer, tetramer, or oligomer when on a cell associated with a disease state and a monomer or dimer when on a cell that is not associated with a disease state.

82. The single chain polypeptide of any one of claims 70-81, wherein the target cell antigen is part of a cell signaling complex when on a cell associated with a disease state and not part of the same cell signaling complex when on a cell that is not associated with a disease state.

83. The single chain polypeptide of any one of claims 70-82, wherein the target cell antigen is part of a cell signaling complex when on a cell associated with a disease state and not part of a cell signaling complex when on a cell that is not associated with a disease state.

84. The single chain polypeptide of any one of claims 70-83, wherein the target cell antigen comprises CD33, FAP, EGFR, HER2, or EpCAM.

85. The single chain polypeptide of any one of claims 71-84, wherein the cell associated with a disease state is a myeloid-cell, fibroblast, or cancer cell.

86. A pharmaceutical composition comprising:
(a) the single chain polypeptide of any one of claims 1-85; and
(b) a pharmaceutically acceptable excipient.

87. An isolated recombinant nucleic acid encoding a single chain polypeptide according any one of claims 1-85.

88. A vector comprising an isolated nucleic acid according to claim 87.

89. A host cell comprising an isolated nucleic acid of claim 87 or a vector according to claim 88.

90. A method of treating a cancer comprising administering to a subject in need thereof a single chain polypeptide according to any one of claims 1-85.

91. The method of claim 90, wherein the cancer is a solid tumor cancer.

92. The method of claim 90, wherein the cancer is a hematological cancer.

93. A method of treating an inflammatory disease or condition comprising administering to a subject in need thereof a single chain polypeptide according to any one of claims 1-85.

94. A method of treating an autoimmune disease or condition comprising administering to a subject in need thereof a single chain polypeptide according to any one of claims 1-85.

95. A method of treating a cardiovascular disease or condition comprising administering to a subject in need thereof a single chain polypeptide according to any one of claims 1-85.

96. A method of treating a fibrotic disease or condition comprising administering to a subject in need thereof a single chain polypeptide according to any one of claims 1-85.

97. A method of treating a bacterial infection comprising administering to the subject in need thereof a single chain polypeptide according to any one of claims 1-85.

98. A method of treating a viral infection comprising administering to the subject in need thereof a single chain polypeptide according to any one of claims 1-85.
